(19) United States
(12) Patent Application Publication
(10) Pub. No.: US 2022/0340917 A1
(43) Pub. Date: Oct. 27, 2022

(54) WATERMELON WITH PALE MICROSEEDS

(71) Applicant: RIJK ZWAAN ZAADTEELT EN ZAADHANDEL B.V., De Lier (NL)

(72) Inventors: Rudolph Adrianus VAN HOF, De Lier (NL); Guadalupe SANCHEZ SILVA, De Lier (NL); Emilio SARRIA VILLADA, De Lier (NL)

(21) Appl. No.: 17/740,956

(22) Filed: May 10, 2022

Related U.S. Application Data
(63) Continuation-in-part of application No. PCT/EP2020/083706, filed on Nov. 27, 2020.
Application No. PCT/EP2020/083706 is a continuation-in-part of application No. PCT/EP2019/082784, filed on Nov. 27, 2019.

(51) Int. Cl. C12N 15/82; C12N 9/02

(52) U.S. Cl. CPC C12N 15/825; C12Y 110/03001; C12N 9/0059

(57) ABSTRACT

The present invention relates to a modified watermelon PPO gene, the wild type of which is identified as SEQ ID NO: 1, encoding the protein of SEQ ID NO: 5, or the wild type of which encodes a protein that has at least 90% sequence identity to SEQ ID NO: 5, wherein the modified PPO gene comprises one or more nucleotides replaced, inserted and/or deleted relative to the wild type, and wherein said one or more replaced, inserted and/or deleted nucleotides result in an absence of functional PPO protein. The present invention further relates to a watermelon plant which may comprise the modified PPO gene, wherein the homozygous presence of the modified PPO gene confers a pale seed color to the plant. The present invention also relates to methods for selecting, producing or the use of the watermelon plant of the invention.

### RELATED APPLICATIONS AND INCORPORATION BY REFERENCE

This application is a continuation-in-part application of international patent application Serial No. PCT/EP2020/083706 filed 27 Nov. 2020, which published as PCT Publication No. WO 2021/105408 on 3 Jun. 2021, which claims benefit of international patent application Serial No. PCT/EP2019/082784 filed 27 Nov. 2019.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said ASCII copy was created May 10, 2022, is named Y7954-00522SL.txt and is 170,694 bytes in size.

### FIELD OF THE INVENTION

The present invention relates to genes that together impart a pale microseed phenotype to a watermelon plant. Additionally, the invention relates to use of these genes for producing watermelon plants with pale colored seeds with optionally a microseed size, as well as to methods for identifying and selecting a watermelon plant having a pale seed color and methods for identifying and selecting a watermelon plant having a microseed size.

### BACKGROUND OF THE INVENTION

Watermelon belongs to the genus Citrullus which is part of the Cucurbit family (Cucurbitaceae). The modern cultivated watermelon is known as Citrullus lanatus var. lanatus (Thunb.) Matsum. & Nakai. Watermelon is grown throughout the tropical and sub-tropical regions of the world, predominantly for consumption of its sweet flesh. The Southern part of the USA, China, the Middle East, Africa, India, Japan and Southern Europe are the most important watermelon producing areas.

Cultivated watermelon plants are large annual plants with a vine-like growth habit. The fruit flesh of mature watermelon fruits of cultivated watermelon is usually red and sweet. The seeds of mature fruits of cultivated watermelons are normally dark (brown to black) and big, making the seeds stand out in the red fruit flesh.

Consumers prefer seedless watermelon fruits. For this reason cultivated watermelon varieties are often triploid. If a triploid watermelon plant is pollinated this triggers fruit development. The three sets of chromosomes make successful meiosis very unlikely however, and cause the ovules or embryos to abort without producing mature seeds, an example of stenospermocarpy. Though the fruits of triploid watermelon plants are considered seedless they do contain such abortive incompletely developed seeds. Triploid hybrid varieties are produced by crossing a tetraploid mother line with a diploid father line. Seed production and the breeding of triploid watermelon varieties is complicated and expensive. As a triploid plant has no viable pollen it is necessary for the watermelon grower to plant a diploid (pollenizer) variety in the production field to provide the pollen that stimulates fruit to form. Usually, one row of the diploid pollenizer variety is planted for every two to three rows of triploid watermelon. The pollenizer variety and the triploid variety need to be synchronized so that pollen are produced by the pollenizer at the time the triploid mother can accept them for induction of fruit set. It is difficult to make good combinations, especially since environmental conditions can affect the pollenizer and triploid differently, leading to asynchrony and lowering of the watermelon fruit yield. Usually varieties are chosen that can be distinguished easily so the seeded diploid fruit can be separated from the seedless triploid fruit for harvesting and marketing. Triploid watermelons germinate weakly, are more difficult to grow than diploid watermelons, and usually produce a lower number of fruits. All this makes triploid watermelon fruit production very expensive and complex for watermelon growers. For growers and consumers the presence of the remains of the undeveloped seeds in the fruit can be a problem. Especially under stress conditions, fruits are produced with clearly noticeable remains of incompletely developed seeds or even normally developed seeds that are objectionable to consumers.

Citation or identification of any document in this application is not an admission that such document is available as prior art to the present invention.

### SUMMARY OF THE INVENTION

It is therefore an object of the present invention to avoid using triploid watermelon fruit production.

The invention relates to a modified watermelon POLYPHENOL OXIDASE (PPO) gene, the wild type of which is identified as SEQ ID NO: 1, encoding the protein of SEQ ID NO: 5, or the wild type of which encodes a protein that has at least 90% sequence identity to SEQ ID NO: 5, wherein the modified PPO gene may comprise one or more nucleotides replaced, inserted and/or deleted relative to the wild type, and wherein said one or more replaced, inserted and/or deleted nucleotides result in an absence of functional PPO protein.

The present invention further relates to a watermelon plant which may comprise the modified PPO gene, wherein the homozygous presence of the modified PPO gene confers a pale seed color to the plant. The present invention also relates to methods for selecting, producing or the use of the watermelon plant of the invention.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U. S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### Deposit

Seeds of watermelon (Citrullus lanatus var. lanatus) that are homozygous for both the modified PPO gene comprising an insertion of a T between nucleotides 711 and 712 (711_712insT) of SEQ ID NO: 1, and the deletion on Chromosome 2 corresponding to 13962 bp being deleted between base pair position 29902114 and 29916077 on the Citrullus lanatus 97103_v1, were deposited with the NCIMB Ltd, Ferguson Building, Craibstone Estate, Bucksburn, Aberdeen AB21 9YA, UK on 27 Feb. 2019 under accession number NCIMB 43364.

The deposited seeds do not meet the DUS criteria which are required for obtaining plant variety protection, and can therefore not be considered to be plant varieties.

The Deposits with NCIMB Ltd, under deposit accession number NCIMB 43364 were made and accepted pursuant to the terms of the Budapest Treaty. Upon issuance of a patent, all restrictions upon the deposit will be removed, and the deposit is intended to meet the requirements of 37 CFR §§ 1.801-1.809. The deposit will be irrevocably and without restriction or condition released to the public upon the issuance of a patent and for the enforceable life of the patent. The deposit will be maintained in the depository for a period of 30 years, or 5 years after the last request, or for the effective life of the patent, whichever is longer, and will be replaced if necessary during that period.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings.

FIG. 1: Mature fruit of a watermelon (Citrullus lanatus var. lanatus) plant that is homozygous for both the modified PPO gene and the deletion on Chromosome 2 corresponding to 13962 bp being deleted between base pair position 29902114 and 29916077 on the Citrullus lanatus 97103_v1 and has red fruit flesh and pale colored microseeds.

FIG. 2: Seeds as deposited at the NCIMB under deposit Accession number 43364 with a pale color and a microseed size (left), and seeds of a wild type watermelon variety that are black and have a big seed size (right). The size bar indicates a size of 1 cm.

### DETAILED DESCRIPTION OF THE INVENTION

In angiosperms, seed development begins with double fertilization. One of the two sperm cells fuses with the egg cell to form the diploid zygote, which then develops into an embryo with a shoot meristem, cotyledons, hypocotyl, root and a root meristem. The other sperm cell fertilizes the diploid central cell to generate the triploid endosperm. In most dicots such as Arabidopsis thaliana, the endosperm grows rapidly initially, but is consumed at later developmental stages. The embryo therefore occupies most of the mature seed. After fertilization the maternal integuments surrounding the developing embryo and endosperm undergo cell differentiation, may accumulate pigments, mucilage and starch granules, and eventually form the mature seed coat.

The seed coat in many species contains dark (brown to black) pigments. Seed coat coloring has been studied best in Arabidopsis thaliana. In Arabidopsis seeds, pigmentation of the seed coat is observed at late stages of seed development. The actual synthesis of the pigments, which are called proanthocyanidins (PA) or condensed tannins, starts during early stages of embryo development (1-2 days after fertilization). These flavonoids initially accumulate as colorless compounds in vacuoles of the endothelium, the innermost cell layer of the integuments, and are oxidized during seed desiccation thereby conferring the brown color to mature seeds. Several Arabidopsis seed coat pigmentation mutants are known. In these so called transparent testa mutants the Arabidopsis seed coat exhibits a white to pale yellow color. Many TRANSPARENT TESTA genes encode enzymes in the flavonoid biosynthesis pathway, while others encode regulatory genes involved in several points of the pathway.

The size of a seed is determined by the coordinated growth of the embryo, endosperm and maternal tissue. Growth of plant seeds up to their species-specific size is predominantly determined by internal developmental signals from maternal and zygotic tissues. Several genes that promote endosperm growth have been identified in Arabidopsis. Loss-of-function mutants of such genes form small seeds. The phenotype of these mutants is determined by the genotype of the zygotic tissues. In contrast, other genes have been identified that act maternally to regulate seed size. These genes are involved in regulating cell proliferation and/or expansion in the maternal integuments. These maternal integuments surrounding the ovule form the seed coat after fertilization, and are thought to set an upper limit to seed size as they provide the cavity for the growth of the embryo and the endosperm.

In the research that led to the present invention, a modification in a POLYPHENOL OXIDASE gene, abbreviated herein as PPO, of watermelon was found to result in the plant comprising the modified PPO gene to have seeds with a pale seed color. Moreover, a non-functional HOOKLESS1 (HLS1) gene and/or a non-functional BCL-2 ASSOCIATED ANTHANOGENE 4 (BAG4) was found to result in the plant comprising said non-functional gene(s) to have seeds with a microseed size. Combining the modified PPO gene and the non-functional HLS1 gene and/or non-functional BAG4 gene in a watermelon plant resulted in a novel watermelon plant producing seeds with a pale seed color and a microseed size. Such a watermelon plant produces fruits that to a consumer seem seedless, without all the disadvantages of triploid watermelon fruit production and breeding.

Polyphenol oxidase (PPO) is an enzyme that catalyzes the hydroxylation of monophenols into ortho-diphenols (cresolase activity) and the oxidation of o-diphenols into o-quinones (catecholase activity). While the biochemical reactions catalyzed by PPOs are well known, data on physiological functions of the enzyme are scarce. The enzyme is present in nearly all plants, and is also found in fungi, bacteria and animals. Most plants and fungi carry multiple PPO type gene copies and their expression is thought to be tissue specific and developmentally controlled or stress-induced. Different copies within a plant have different expression profiles and even their cellular localization may differ. Plant PPO proteins are best known for causing the rapid polymerization of o-quinones to produce black, brown or red pigments (polyphenols) that cause e.g. fruit or vegetable browning upon damage of the tissue through bruising or cutting. A function of PPOs in resistance to pathogens and herbivores has also been proposed in some plants. Several assays exist to measure PPO enzyme activity.

The watermelon genome comprises 8 PPO type gene copies that are all arranged in tandem on chromosome 3 (Citrullus lanatus 97103 Chr3:5634000-5814000, see Guo et al, 2013, The draft genome of watermelon (Citrullus lanatus) and resequencing of 20 diverse accessions. Nature Genetics 45(1):51-58).

The present invention provides a modified watermelon PPO gene, which is one of the above mentioned eight gene copies, the wild type of which is identified as SEQ ID NO: 1, encoding the protein of SEQ ID NO: 5, or the wild type of which encodes a protein that has at least 90% sequence identity to SEQ ID NO: 5, wherein the modified PPO gene may comprise one or more nucleotides replaced, inserted and/or deleted relative to the wild type, and wherein said one or more replaced, inserted and/or deleted nucleotides result in an absence of functional PPO protein.

Suitably, sequence identity is calculated using the Sequence Identities and Similarities (SIAS) tool, which can be accessed at imed.med.ucm.es/Tools/sias.html. SIAS calculates pairwise sequence identity and similarity percentages between each pair of sequences from a multiple sequence alignment. Sequence identity is calculated using a method taking the gaps into account; sequence similarity is calculated based on grouping of amino acids having similar properties. For calculations, default settings for SIM percentage, similarity amino acid grouping, sequence length, normalized similarity score, matrix and gap penalties are used.

The DNA sequence of a gene may be altered in a number of ways, and will have varying effects depending on where the modification(s) occur and whether they alter the expression level and/or function of the encoded protein. Examples of DNA modifications include an insertion, a deletion, and base substitution (also called nucleotide replacement), this may e.g. result in a frameshift mutation, a nonsense mutation, a null-mutation, a knockout mutation, a premature stop codon, and/or an amino acid substitution.

An insertion changes the number of DNA bases in a gene by adding a piece of DNA. A deletion changes the number of DNA bases by removing one or more base pairs, or even an entire gene or neighboring genes. These types of modifications may alter the function of the resulting protein.

Frame shift mutations are caused by insertion or deletion of one or more base pairs in a DNA sequence encoding a protein. When the number of inserted or deleted base pairs at a certain position within the coding sequence is not a multiple of 3, the triplet codon encoding the individual amino acids of the protein sequence becomes shifted relative to the original open reading frame, and then the encoded protein sequence changes dramatically. Protein translation will result in an entirely different amino acid sequence than that of the originally encoded protein, and very often a frameshift leads to a premature stop codon in the open reading frame. The overall result is that the encoded protein no longer has the same biological function as the originally encoded protein.

An amino acid substitution in an encoded protein sequence arises when the mutation or base substitution of one or more base pairs in the coding sequence results in an altered triplet codon, often encoding a different amino acid. Mutations resulting in an amino acid substitution are called non-synonymous or missense mutations. Due to the redundancy of the genetic code not all point mutations lead to amino acid changes. Such mutations are termed silent mutations. Some amino acid changes are conservative, i.e. they lead to the replacement of one amino acid by another amino acid with comparable properties, such that the mutation is unlikely to dramatically change the folding of the mature protein, or influence its function. Other amino acid changes are more likely to affect protein function: non-conservative amino acid changes in domains that play a role in substrate recognition, the active site of enzymes, interaction domains or in major structural domains (such as transmembrane helices) may partly or completely destroy the functionality of an encoded protein, without thereby necessarily affecting the expression level of the encoding gene. Whether an amino acid substitution is conservative or non-conservative may be predicted on the basis of chemical properties, for example similarity in polarity, charge, solubility, hydrophobicity, hydrophilicity or amphipathic nature of the amino acids.

A deletion, insertion, frame shift mutation and/or amino-acid substitution may result in a nonsense mutation. A nonsense mutation is a mutation in a nucleic acid molecule encoding a protein whereby a codon is changed into a premature stop codon. Converting an amino acid into a premature stop codon results in a truncated protein. How much of the protein is lost determines whether or not the protein is still functional. Especially when all or part of the conserved functional domains are lacking from the truncated protein it is likely protein function is affected. Premature stop codons may also lead to nonsense-mediated decay, in which mRNAs that are transcribed from an allele carrying a nonsense mutation are eliminated, leading to low RNA expression levels and no or very little protein.

A deletion, insertion, frame shift mutation and/or amino-acid substitution may result in a null mutation or knockout mutation. A null mutation or knockout mutation is a mutation that eliminates the function of the affected gene. For example, a null mutation in a gene that usually encodes a specific enzyme leads to the production of a nonfunctional enzyme or no enzyme at all.

The wild type of the PPO gene of this invention may comprise SEQ ID NO: 1. In the publicly available genome assembly of Citrullus lanatus cv. 97103 (version 1, see Guo et al, 2013, The draft genome of watermelon (Citrullus lanatus) and resequencing of 20 diverse accessions. Nature Genetics 45(1):51-58) said wild type of the modified PPO gene of this invention is located on chromosome 3 at position 5704673 . . . 5707416 (-). SEQ ID NO: 20 provides the reverse complementary sequence of the PPO gene that is present on the positive strand. Also encompassed by the term “wild type of the PPO gene of this invention” is a gene that has, in order of increased preference, at least 90%, 91%, 92%, 93%, 94%, 95%, 96%, 97%, 98% or 99% sequence identity to SEQ ID NO:1.

The wild type of the PPO gene of the invention encodes the protein of SEQ ID NO: 5. This wild type PPO protein may comprise the following conserved domains: Tyrosinase domain (aa 171-378 of SEQ ID NO: 5, Pfam domain PF00264), PPO1-DWL domain (aa 384-432 of SEQ ID NO: 5, Pfam domain PF12142), PPO1-KFDV domain (aa 458-585 of SEQ ID NO: 5, Pfam domain PF12143). Also encompassed by the term “wild type of the PPO gene of this invention” is a gene that encodes a protein that has, in order of increased preference, at least 90%, 91%, 92%, 93%, 94%, 95%, 96%, 97%, 98% or 99% sequence identity to SEQ ID NO: 5.

The modified PPO gene of the invention may comprise one or more nucleotides replaced, inserted and/or deleted relative to the wild type, and said one or more replaced, inserted and/or deleted nucleotides result in an absence of functional PPO protein. In the context of this invention the term “absence of functional PPO protein” means that either no PPO protein is expressed, or that PPO protein is expressed that is non-functional and does not have PPO enzyme activity. The modification to the PPO gene can lead to the absence of PPO RNA or a significantly decreased PPO RNA level, resulting in an absence of PPO protein. Alternatively, the modified PPO protein is expressed but is non-functional: an absence of one or more of the functional domains of the PPO protein results in a modified PPO protein that cannot perform its function as a polyphenol oxidase enzyme. The absence of functional PPO protein can e.g. be determined by using a PPO enzyme activity assay. In short, in such an assay a protein extract is made of the seed coat tissue, after which different phenolic substrates can be added to this protein extract and a significant reduction of PPO activity can be determined by measuring the color change using a spectrophotometer (see in general e.g. Rocha et al, 1998, Characterisation of ‘Starking’ apple polyphenoloxidase. Journal of the Science of Food and Agriculture 77(4):527-534). An even simpler method is by putting the entire seed coat or seed in a phenolic substrate and checking for a color change, such as is described in Chen et al. (2014, Fine-mapping and candidate gene analysis of BLACK HULL1 in rice (Oryza sativa L.). Plant Omics, 7(1):12-18).

In one embodiment, the modified PPO gene of the invention may comprise a premature stop codon that leads to an absence of functional PPO protein. In another embodiment, the modified PPO gene of the invention may comprise a premature stop codon resulting in the absence of the PPO1-KFDV domain from the encoded modified PPO protein, the absence of the PPO1-KFDV and the PPO1-DWL domain from the encoded modified PPO protein, or the absence of the PPO1-KFDV, the PPO1-DWL and the Tyrosinase domain from the encoded modified PPO protein. In a preferred embodiment, the one or more nucleotides that are replaced, inserted and/or deleted in the modified PPO gene of the invention relative to the wild type are at position 1 to 712 of SEQ ID NO: 1, resulting in a premature stop codon that leads to an absence of functional protein. In a most preferred embodiment, the modified PPO gene may comprise an insertion of a T between nucleotides 711 and 712 (711_712insT) of SEQ ID NO: 1.

In the genome of a watermelon plant representative seed of which was deposited under accession number NCIMB 43364 there is an insertion of an A at the genomic position corresponding to cl_97103_v1_Chr3:5706705 (Guo et al, supra). This insertion leads to a frameshift, which leads to the introduction of a premature stop codon in the PPO gene of SEQ ID NO: 1 (which gene is in the genome on the reverse strand). SEQ ID NO: 20 provides the reverse complementary sequence of the PPO gene that is present on the positive strand. The modified PPO gene may comprise an insertion of a T between nucleotides 711 and 712 of SEQ ID NO: 1. This one base pair insertion leads to a frameshift, which leads to 13 amino acids being encoded in the wrong frame followed by a premature stop codon at position 751-753 of the modified PPO gene (SEQ ID NO:2). Whereas the size of the wild type PPO protein is 587 amino acids (SEQ ID NO:5), the modified PPO protein (SEQ ID NO:6), if produced at all, is only 250 amino acids long, may comprise only a small part of its Tyrosinase domain, lacks its conserved PPO1-DWL and PPO1-KFDV domains completely and may comprise 13 altered amino acids at its C-terminus. The mutant protein is thus non-functional.

The modified PPO gene of this invention confers a pale seed color to the plant when present homozygously.

In one embodiment, the modified PPO gene of this invention is a nucleic acid, in particular a nucleic acid molecule, more in particular an isolated nucleic acid molecule.

Seed color can be determined visually. While the color of fully developed and mature dried watermelon seeds of cultivated watermelon plants not carrying the modified PPO gene of the invention normally varies from middle brown to black depending on the variety, fully developed and mature dried seeds of cultivated watermelon plants carrying the modified PPO gene of the invention homozygously may be indicated as beige, light yellow, pale yellow, wheat, or light khaki. Seed color hardly changes upon the drying of the fresh wet seeds as they are present in the mature watermelon fruit. The seed color of fully developed and mature fresh seeds of cultivated watermelon plants carrying the modified PPO gene of the invention homozygously may thus be indicated as beige, light yellow, pale yellow, wheat, or light khaki. When comparing the color of seeds produced by plants of the invention carrying the modified PPO gene of the invention homozygously and seeds of isogenic plants carrying the modified PPO gene either heterozygously or not at all, all seeds have to be at the same developmental stage and all seeds have to be either all fresh or all dried.

An RHS color chart (The Royal Horticultural Society, London, UK) is often used by plant breeders and growers for determining plant colors visually, however, it is clear the color may also be determined using other color charts or systems. Colors may, for example, also be specified in RGB color codes, using the Munsell color system or may be determined using a colorimeter or image analysis. The skilled person knows how to use these different color systems and convert color codes between different color systems.

The color of seeds can also be determined by using a colorimeter or by using image analysis, e.g. as described in Example 1. When determining the color of seeds it is good to do this on an appropriate number of seeds, such as at least 10 seeds, from each seed lot, so that the average color values can be calculated. For image analysis photographs need to be taken in a standardized set-up. It is important that the about 10 seeds to be photographed are clearly separated from each other and for later color correction of the photographs it is good to include a colorchart, such as the X-rite colorchecker passport colorchart, in each picture. By image analysis of the color corrected photographs, using a CellProfiler pipeline or a comparable program, calibrated RGB values can be generated. These can then be translated into, for example, CIELAB L*a*b* color values using a color calibration algorithm.

A color scale that is widely used to measure colors, for instance using a colorimeter or image analysis, is the CIELAB color scale. The scale includes 3 data variables: L*, a* and b*. L* indicates lightness on a 0 to 100 scale, where 0 is black and 100 is white. The variables a* and b* indicate the amount of red, green, blue and yellow color: a* value indicates color change from green (negative values) to red (positive values), while b* indicates color change from yellow (positive values) to blue (negative values). Differences in color between two samples can be expressed in terms of change in L* and/or a*, and/or b*.

Seeds produced by plants carrying the modified PPO gene of the invention homozygously have a pale seed color. As used herein the term “pale seed color” is intended to refer to a seed color of fully developed and mature dry seeds that is beige, light yellow, pale yellow or light khaki and/or the fully developed and mature dry seeds having an L* (10° /D65) score when determined using image analysis, e.g. as described above or in Example 1, of at least, in order of increased preference 55, 60, 62, 64, 66, 68, 69, 70, 71, 72, 73, 74, 75, 76, 77, 78, 80, 85, 90. The L* (10° /D65) score when determined using image analysis on dry mature seeds of said plant is suitably not higher than 99.

The pale seed color phenotype of seeds of the invention is due to the reduction or absence of brown pigments in the seed coat, also called the testa. The seed coats of the pale colored seeds of the invention have a reduced amount of the brown pigments that are normally present in the seed coats of seeds at the mature seed stage produced by plants not comprising the modified PPO gene of the invention homozygously. In particular, the seed coats of seeds of this invention have such a low amount of the brown pigments that the brown color in the seed coats of said seeds of plants of the invention is not detectable by the eye. More in particular, seed coats of seeds of this invention completely lack the brown pigments that are normally present in the seed coats of brown or black seeds.

The seed coat is the outer protective layer of the seed and is derived from the integuments of the ovule. The seed coat is thus of maternal origin. The color of the seeds therefore is determined by the genotype of the plant that produces the seeds (the mother plant that receives pollen in a cross). Since this trait is recessive, the watermelon plant producing the seeds (mother plant) needs to comprise the modified PPO gene of the invention homozygously to produce pale seeds. The genotype of the father plant providing the pollen in the cross has no impact on the color of the seeds produced by the mother plant after this pollination.

The invention relates to a watermelon plant which may comprise the modified PPO gene of the invention, wherein the homozygous presence of the modified PPO gene confers a pale seed color to the plant. The modified PPO gene of the invention can be as comprised in the genome of a Citrullus lanatus var. lanatus plant representative seed of which was deposited under accession number NCIMB 43364. The plant can comprise the modified PPO gene of the invention heterozygously, in which case the seeds produced by the plant do not have the pale seed color trait but the plant is useful for transferring the modified PPO gene of the invention to another plant. The plant can also comprise the modified PPO gene of the invention homozygously, in which case said plant produces seeds with a pale seed color.

This invention further relates to a watermelon plant which may comprise the modified PPO gene of the invention, wherein the plant further may comprise a non-functional HLS1 gene, the wild type of which is identified as SEQ ID NO: 7 encoding the protein of SEQ ID NO: 9, or the wild type of which encodes a protein that has at least 90% sequence identity to SEQ ID NO: 9, and/or a non-functional BAG4 gene, the wild type of which is identified as SEQ ID NO: 10 encoding the protein of SEQ ID NO: 12, or the wild type of which encodes a protein that has at least 90% sequence identity to SEQ ID NO: 12, wherein the absence of functional HLS1 protein and/or the absence of functional BAG4 protein confers a microseed size to the plant.

The wild type of the watermelon HLS1 gene of this invention may comprise SEQ ID NO: 7. In the publicly available genome assembly of Citrullus lanatus cv. 97103 (version 1, see Guo et al, supra) said wild type HLS1 gene is located on chromosome 2 at position 29904246 . . . 29906227 (−). Also encompassed by the term wild type of the HLS1 gene of this invention is a gene that has, in order of increased preference, at least 90%, 91%, 92%, 93%, 94%, 95%, 96%, 97%, 98% or 99% sequence identity to SEQ ID NO: 7.

The wild type of the watermelon HLS1 gene of this invention encodes the protein of SEQ ID NO: 9. This wild type HLS1 protein may comprise the following conserved domain: Acetyltransferase (GNAT) family domain (aa 38-146 of SEQ ID NO: 9, Pfam domain pfam00583). This HLS1 gene is an N-acetyltransferase family gene which encodes an enzyme that catalyzes the transfer of an acetyl group to a substrate. The Arabidopsis HLS1 gene was linked to regulation of apical hook formation under etiolation and ethylene treatment, and was shown to be involved in sugar and auxin signaling. The Arabidopsis HLS1 gene was shown to function through histone acetylation (Liao et al, 2016, Arabidopsis HOOKLESS1 Regulates Responses to Pathogens and Abscisic Acid through Interaction with MED18 and Acetylation of WRKY33 and ABI5 Chromatin. The Plant Cell, 28 (7): 1662-1681). Also encompassed by the term “wild type of the HLS1 gene of this invention” is a gene that encodes a protein that has, in order of increased preference, at least 90%, 91%, 92%, 93%, 94%, 95%, 96%, 97%, 98% or 99% sequence identity to SEQ ID NO: 9.

The non-functional HLS1 gene of the invention can comprise one or more nucleotides replaced, inserted and/or deleted relative to the wild type resulting in an absence of functional HLS1 protein. In this context, the absence of functional HLS1 protein can be due to the absence of HLS1 RNA resulting in an absence of HLS1 protein. The absence of functional HLS1 protein can also mean an absence of the functional domain of the HLS1 protein, resulting in a modified HLS1 protein that cannot perform its function. The HLS1 gene of the invention can also be non-functional because it is absent from the genome.

In one embodiment, the non-functional HLS1 gene of this invention is a nucleic acid, in particular a nucleic acid molecule, more in particular an isolated nucleic acid molecule.

The wild type of the watermelon BAG4 gene of this invention may comprise SEQ ID NO: 10. In the publicly available genome assembly of Citrullus lanatus cv. 97103 (version 1, see Guo et al, supra) said wild type BAG4 gene is located on chromosome 2 at position 29911929 . . . 29915565 (+). Also encompassed by the term “wild type of the BAG4 gene of this invention” is a gene that has, in order of increased preference, at least 90%, 91%, 92%, 93%, 94%, 95%, 96%, 97%, 98% or 99% sequence identity to SEQ ID NO: 10.

The wild type of the watermelon BAG4 gene of this invention encodes the protein of SEQ ID NO: 12. This wild type BAG4 protein may comprise the following conserved domains: ubiquitin-like domain (aa 49-117 of SEQ ID NO: 12, InterPro domain IPR000626) and BAG-domain (aa 141-219 of SEQ ID NO:12, InterPro domain IPR003103). The protein encoded by the BAG4 gene is a member of the BAG1-related protein family. BAG1 is an anti-apoptotic protein that functions through interactions with a variety of cell apoptosis and growth related proteins. Also encompassed by the term “wild type of the BAG4 gene of this invention” is a gene that encodes a protein that has, in order of increased preference, at least 90%, 91%, 92%, 93%, 94%, 95%, 96%, 97%, 98% or 99% sequence identity to SEQ ID NO: 12.

The non-functional BAG4 gene of the invention can comprise one or more nucleotides replaced, inserted and/or deleted relative to the wild type resulting in an absence of functional BAG4 protein. In this context, the absence of functional BAG4 protein can be due to the absence of BAG4 RNA resulting in an absence of BAG4 protein. The absence of functional BAG4 protein can also mean an absence of one or more or all of the functional domains of the BAG4 protein, resulting in a modified BAG4 protein that cannot perform its function. The BAG4 gene of the invention can also be non-functional because it is absent from the genome.

In one embodiment the non-functional BAG4 gene of this invention is a nucleic acid, in particular a nucleic acid molecule, more in particular an isolated nucleic acid molecule.

The watermelon plant of the invention can comprise the non-functional HLS1 gene and/or the non-functional BAG4 gene heterozygously. Preferably, a watermelon plant of the invention homozygously may comprise the non-functional HLS1 gene and/or homozygously may comprise the non-functional BAG4 gene and the plant produces seeds with a microseed size. If the HLS1 gene and/or the BAG4 gene are absent from the genome, this absence is preferably also homozygous, which means that both copies are absent.

The invention further relates to a watermelon plant which may comprise the modified PPO gene of the invention, which may further comprise a deletion on chromosome 2 corresponding to 13962 bp being deleted between base pair position 29902114 and 29916077 on the Citrullus lanatus 97103_v1 genome, wherein this deletion confers a microseed size to the plant when present homozygously. By this deletion all nucleotides starting from the G at position 29902115 on chromosome 2 of the Citrullus lanatus 97103_v1 genome to the A at position 29916076 on chromosome 2 of the Citrullus lanatus 97103_v1 genome, have been deleted. Sequence SEQ ID NO:13 provides the cl_97103_v1 genomic sequence from position 29897185 to 29920517 of chromosome 2. The genomic deletion conferring microseed size corresponds to a deletion of all nucleotides between base pair position 4930 and 18893 of SEQ ID NO: 13. This genomic deletion leads to two genes being deleted: the HLS1 gene of SEQ ID NO: 7 and the BAG4 gene of SEQ ID NO: 10. Preferably, this deletion is as comprised in the genome of a Citrullus lanatus var. lanatus plant representative seed of which was deposited under accession number NCIMB 43364. This deletion can be present heterozygously, in which case the seeds produced by the plant do not have the microseed size trait but the plant is useful for transferring this deletion of the invention to another plant. Preferably, this deletion is present homozygously and the plant produces seeds with a microseed size.

The absence of functional HLS1 protein and/or the absence of functional BAG4 protein confers a microseed size to the watermelon plant.

Seed size can be estimated visually by a skilled person, but is better measured using image analysis or using a caliper as described in Example 1. When determining the size of seeds this has to be done on an appropriate number of fully developed and mature dry seeds, such as at least 10 seeds, from each seed lot, so that the average size can be calculated. With a caliper seed length, seed width and seed thickness can be measured. Seed length is the best measure for watermelon seed size.

Seeds as deposited at the NCIMB under deposit Accession number 43364 with a pale color and a microseed size have an average length of 4.0 mm, an average width of 2.5 mm and an average thickness of 1.5 mm. The average 100 seed weight (100SDW, in g) of seeds as deposited is 0.7 g. In general there is a strong correlation between seed length and 100SDW.

As used herein the term “microseed size” is intended to refer to fully developed and mature dry seeds having an average length when determined on about 10 seeds, of at most, in order of increased preference 6.0 mm, 5.9 mm, 5.8 mm, 5.7 mm, 5.5 mm, 5.4 mm, 5.3 mm, 5.2 mm, 5.1 mm, 5.0 mm, 4.9 mm, 4.8 mm, 4.7 mm, 4.6 mm, 4.5 mm, 4.4 mm, 4.3 mm, 4.2 mm, 4.1 mm, 4.0 mm, 3.9 mm, 3.8 mm, 3.7 mm, 3.6 mm, 3.5 mm, 3.0 mm, 2.5 mm, or 2.1 mm. The seed length is suitable not lower than 2.0 mm.

As used herein the term “watermelon plant of the invention” or “plant of the invention” is intended to refer to a watermelon (Citrullus lanatus var. lanatus) plant which may comprise the modified PPO gene of the invention and optionally may further comprise the non-functional HLS1 gene of the invention and/or the non-functional BAG4 gene of the invention. A watermelon plant which may comprise the modified PPO gene of the invention and may further comprise a deletion on chromosome 2 corresponding to 13962 bp being deleted between base pair position 29902114 and 29916077 on the Citrullus lanatus 97103_v1 genome, is also a plant of the invention as in this plant both the HLS1 gene and the BAG4 gene are absent from the genome. Preferably in the plant of the invention said deletion is as comprised in the genome of a Citrullus lanatus var. lanatus plant representative seed of which was deposited under accession number NCIMB 43364.

The watermelon plant of the invention can be a watermelon plant of any type, any fruit form or fruit color, and is preferably an agronomically elite watermelon plant. In one embodiment, the mature fruits of the watermelon plant of the invention have red, orange or yellow flesh. In another embodiment, the mature fruits of said plant have flesh with soluble solids of at least, in order of increased preference, 5.0 degrees Brix, 6.0 degrees Brix, 7.0 degrees Brix, 8.0 degrees Brix, 9.0 degrees Brix, 9.5 degrees Brix, 10.0 degrees Brix, 10.5 degrees Brix, 11.0 degrees Brix, 11.5 degrees Brix, 12.0 degrees Brix, 12.5 degrees Brix, 13.0 degrees Brix, 13.5 degrees Brix, 14.0 degrees Brix, 14.5 degrees Brix, 15.0 degrees Brix, 15.5 degrees Brix, 16.0 degrees Brix, or 17.0 degrees Brix. The soluble solids of the mature fruits of said plant are suitably not higher than 18 degrees Brix. In another embodiment the watermelon plant of the invention is a plant of an inbred line or a hybrid plant. In yet another embodiment the watermelon plant of the invention is a diploid, tetraploid or triploid plant.

In case triploid watermelon plants homozygously comprise the modified PPO gene of the invention and optionally further homozygously comprise the non-functional HLS1 gene of the invention and/or the non-functional BAG4 gene of the invention, the fruits these plants produce after being pollinated by a diploid pollenizer are improved over triploid watermelon plants not containing the gene(s) of this invention. The incompletely developed seeds or occasional normally developed seeds that can be present in such fruits are less noticeable than in normal triploid fruits because of the pale color and optionally the smaller size.

In the context of this invention an “agronomically elite watermelon” plant is a plant having a genotype that results in an accumulation of distinguishable and desirable agronomic traits which allow a producer to harvest a product of commercial significance.

As used herein, a “plant of an inbred line” is a plant of a population of plants that is the result of three or more rounds of selfing, or backcrossing, or which plant is a doubled haploid. An inbred line may e.g. be a parent line used for the production of a commercial hybrid.

As used herein, a “hybrid plant” is a plant which is the result of a cross between two different plants having different genotypes. More in particular, a hybrid plant is the result of a cross between plants of two different inbred lines, such that a hybrid plant may e.g. be a plant of an F₁ hybrid variety.

The invention also encompasses a watermelon seed, which may comprise the modified PPO gene of the invention, wherein the plant grown from said seed produces seeds with a pale seed color as a result of the homozygous presence of the modified PPO gene, and optionally may further comprise the non-functional HLS1 gene of the invention and/or the non-functional BAG4 gene of the invention, wherein the absence of functional HLS1 protein and/or the absence of functional BAG4 protein confers a microseed size to the plant grown from said seed.

The invention further relates to a part of the watermelon plant of the invention, which may comprise a fruit of the plant of the invention or a seed of the plant of the invention, wherein the plant part may comprise the modified PPO gene of the invention and optionally further may comprise the non-functional HLS1 gene of the invention and/or the non-functional BAG4 gene of the invention.

The invention further relates to a watermelon fruit produced by the watermelon plant of the invention, wherein the watermelon fruit has seeds that have a pale seed color and optionally a microseed size. This watermelon fruit is a fruit of the invention.

Moreover, the invention also relates to a food product or a processed food product which may comprise the fruit of the invention or a part thereof. The food product may have undergone one or more processing steps. Such a processing step might comprise but is not limited to any one of the following treatments or combinations thereof: peeling, cutting, washing, juicing, cooking, cooling or preparing a salad mixture which may comprise the fruit of the invention. The processed form that is obtained is also part of this invention since it may comprise DNA in which the modified PPO gene and/or a non-functional HLS1 gene and/or a non-functional BAG4 gene are present.

The invention further relates to a cell of a plant of the invention. Such a cell may either be in isolated form or a part of the complete plant or parts thereof and still constitutes a cell of the invention because such a cell harbors the genetic information that imparts the pale seed color and optionally the microseed size to a plant of the invention. Each cell of a plant of the invention carries the genetic information that leads to the pale seed color and optionally the microseed size of the invention. A cell of the invention may also be a regenerable cell that can regenerate into a new plant of the invention. The presence of genetic information as used herein is the presence of the modified PPO gene of the invention and optionally the presence of the non-functional HLS1 gene of the invention and/or the non-functional BAG4 gene of the invention, or the presence of the deletion on chromosome 2 as defined herein.

The invention further relates to plant tissue of a plant of the invention, which may comprise the modified PPO gene of the invention, and optionally further may comprise the non-functional HLS1 gene of the invention and/or the non-functional BAG4 gene of the invention. The tissue can be undifferentiated tissue or already differentiated tissue. Undifferentiated tissue is for example a stem tip, an anther, a petal, or pollen, and can be used in micropropagation to obtain new plantlets that are grown into new plants of the invention. The tissue can also be grown from a cell of the invention.

The invention moreover relates to progeny of a plant, a cell, a tissue, or a seed of the invention, which progeny may comprise the modified PPO gene of the invention, and optionally further may comprise the non-functional HLS1 gene of the invention, and/or the non-functional BAG4 gene of the invention. Such progeny can in itself be a plant, a cell, a tissue, or a seed. The progeny can in particular be progeny of a plant of the invention deposited under NCIMB Accession number 43364. As used herein “progeny” is intended to mean the first and all further descendants from a cross with a plant of the invention, wherein a cross may comprise a cross with itself or a cross with another plant, and wherein a descendant that is determined to be progeny may comprise the modified PPO gene of the invention, and optionally further may comprise the non-functional HLS1 gene of the invention and/or the non-functional BAG4 gene of the invention. Progeny also encompasses material that is obtained by vegetative propagation or another form of multiplication. Preferably, the progeny plant produces seeds that have a pale seed color as a result of the homozygous presence of the modified PPO gene of the invention, and optionally a microseed size as a result of the presence of the non-functional HLS1 gene of the invention and/or the non-functional BAG4 gene of the invention, or the presence of the deletion on chromosome 2 as defined herein.

The invention also relates to propagation material capable of developing into and/or being derived from a plant of the invention, wherein the propagation material may comprise the modified PPO gene of the invention, and optionally further may comprise the non-functional HLS1 gene of the invention and/or the non-functional BAG4 gene of the invention, and wherein the propagation material is selected from a group consisting of a microspore, a pollen, an ovary, an ovule, an embryo, an embryo sac, an egg cell, a cutting, a root, a root tip, a hypocotyl, a cotyledon, a stem, a leave, a flower, an anther, a seed, a meristematic cell, a protoplast and a cell, or a tissue culture thereof.

The invention further relates to use of the modified PPO gene of the invention for producing a plant that produces seeds with a pale seed color. The plant that produces seeds with a pale seed color may be produced by introduction of the modified PPO gene into its genome, in particular by means of mutagenesis or introgression, or combinations thereof. The seeds of said plant may have a microseed size.

The invention further relates to use of the non-functional HLS1 gene of the invention and/or the non-functional BAG4 gene of the invention for producing a plant that produces seeds with microseed size. The plant that produces seeds with a microseed size may be produced by introduction of the non-functional HLS1 gene of the invention and/or the non-functional BAG4 gene of the invention into its genome, in particular by means of mutagenesis or introgression, or combinations thereof. Deleting the HLS1 gene and/or BAG4 gene from the genome can also lead to the HLS1 gene of the invention and/or the BAG4 gene of the invention being non-functional. The seeds of said plant may have a pale color.

The invention also relates to use of the plant of the invention for the production of a watermelon fruit having seeds that have a pale seed color and optionally a microseed size.

The invention further relates to a marker for the identification of a modified PPO gene, wherein the marker sequence detects an insertion of a T between nucleotides 711 and 712 of SEQ ID NO: 1. This insertion corresponds to a single nucleotide insertion of an A at position cl_97103_v1_Chr3:5706705. An example of such a marker is marker CL08381 (SEQ ID NO: 14 and SEQ ID NO:15). SEQ ID NO: 15 represents the allele of marker CL08381 as it is present in the genome of a plant which may comprise the modified PPO gene of this invention. SEQ ID NO:14 represents the wild type allele of this same marker, as is present in genomes of plants that do not comprise the modified PPO gene of this invention. The nucleotide that is different between the two marker alleles of marker CL08381 is underlined and in bold in Table 4 below. The marker allele (SEQ ID NO: 15) for the modified PPO gene has a single nucleotide insertion of an A that is underlined and in bold in Table 2 (position 101 of SEQ ID NO:15).

Use of this marker for identification and/or selection of a watermelon plant producing seeds with a pale seed color is also part of this invention. The invention further relates to a method for selecting a watermelon plant that produces seeds with a pale seed color, which may comprise identifying the presence of a modification in the PPO gene, optionally checking the color of the seeds the plant produces, and selecting a plant that homozygously may comprise said modification as a plant that produces seeds with a pale seed color. The identification of the presence of a modification in the PPO gene may be performed by using the marker as defined above.

The invention further relates to a marker for the identification of a deletion on chromosome 2, wherein the marker sequence detects the presence or absence of a deletion corresponding to 13962 bp being deleted between base pair position 4930 and 18893 of SEQ ID NO: 13. Use of this marker for identification and/or selection of a watermelon plant producing seeds with a microseed size is also part of this invention. Also encompassed in this invention is a method for selecting a watermelon plant that produces seeds with a microseed size, which may comprise identifying the presence of the deletion on chromosome 2 using the marker as defined above, optionally checking the size of the seeds the plant produces, and selecting a plant that homozygously may comprise said deletion as a plant that produces seeds with a microseed size.

An example of a marker for detecting the presence of the deletion is marker CL_chr2_gap1 with primers SEQ ID NO:16 plus SEQ ID NO:17 (see Table 4). In material comprising the genome deletion said primers amplify a PCR product of 446 bp, in material without said genome deletion no PCR product is amplified by these primers. An example of a marker for detecting the absence of the deletion is marker CL_chr2_gap2 with primers SEQ ID NO:18 plus SEQ ID NO:19 (see Table 4). In material not comprising the genome deletion said primers amplify a PCR product of 945 bp, in material comprising said genome deletion no PCR product is amplified by these primers.

Also encompassed in this invention is a method for identifying the presence of the genomic deletion leading to the microseed size, wherein the method may comprise the steps of:

a) running an assay with the primers represented by SEQ ID NO:16 plus SEQ ID NO:17 and/or SEQ ID NO:18 plus SEQ ID NO:19 to determine the presence of an amplification product of SEQ ID NO:16 and SEQ ID NO:17 and/or an amplification product of SEQ ID NO:18 and SEQ ID NO:19;

b) determining the presence of the deletion by assigning: presence of the deletion when the product of the primer represented by SEQ ID NO:16 and the primer represented by SEQ ID NO:17 is produced, and absence of the deletion when the product of the primer represented by SEQ ID NO:18 and the primer represented by SEQ ID NO:19 is produced.

The invention further relates to a method for producing a watermelon plant that produces seeds that have a pale seed color, which may comprise modifying the wild type of the PPO gene of this invention, wherein the modification results in an absence of functional PPO protein, and the absence of functional PPO protein leads to the seeds of the produced plant having a pale seed color. The wild type of the PPO gene of this invention is a gene that has, in order of increased preference, at least 90%, 91%, 92%, 93%, 94%, 95%, 96%, 97%, 98%, 99% or 100% sequence identity to SEQ ID NO:1. Optionally, the plant in which the PPO gene is modified has seeds with a microseed size.

The invention further relates to a method for producing a watermelon plant that produces seeds that have a microseed size, which may comprise modifying the wild type of the HLS1 gene of this invention and/or the wild type of the BAG4 gene of this invention, wherein the modification results in an absence of functional HLS1 protein and/or an absence of functional BAG4 protein in the plant, which leads to the seeds produced by said plant having a microseed size. The wild type of the HLS1 gene of this invention is a gene that has, in order of increased preference, at least 90%, 91%, 92%, 93%, 94%, 95%, 96%, 97%, 98%, 99% or 100% sequence identity to SEQ ID NO:7. The wild type of the BAG4 gene of this invention is a gene that has, in order of increased preference, at least 90%, 91%, 92%, 93%, 94%, 95%, 96%, 97%, 98%, 99% or 100% sequence identity to SEQ ID NO:10. In one embodiment, the modification is a deletion of the HLS1 gene and/or the BAG4 gene. Optionally, the plant in which the HLS1 gene and/or the BAG4 gene is modified has seeds with a pale seed color.

This invention also relates to a modified nucleic acid molecule, the wild type of which is identified as SEQ ID NO: 13, or the wild type of which has at least 90% sequence identity to SEQ ID NO: 13 , wherein the modified nucleic acid does not comprise SEQ ID NO: 7 and/or SEQ ID NO: 10, wherein the modified nucleic acid confers a microseed size to the watermelon plant when present homozygously. In one embodiment, this nucleic acid molecule may comprise a deletion corresponding to 13962 bp being deleted between base pair position 4930 and 18893 of SEQ ID NO: 13.

Moreover, this invention relates to use of said modified nucleic acid molecule for producing a watermelon plant that produces seeds with a microseed size. The watermelon plant that produces seeds with a microseed size may be produced by introduction of the modified nucleic acid molecule into its genome, in particular by means of mutagenesis or introgression, or combinations thereof.

The present invention relates to a method for the production of a watermelon plant that produces seeds that have a pale seed color, said method which may comprise:

a) crossing a plant which may comprise the modified PPO gene of the invention with a plant not comprising said modified PPO gene;

b) optionally performing one or more rounds of selfing and/or crossing a plant resulting from step a) to obtain a further generation population;

c) selecting from the population a plant that homozygously may comprise the modified PPO gene that produces seeds that have a pale seed color.

The present invention also relates to a method for the production of a watermelon plant that produces seeds that have a pale seed color and a microseed size, said method which may comprise:

a) crossing a plant which may comprise the modified PPO gene of the invention and which may comprise the non-functional HLS1 gene of the invention and/or the non-functional BAG4 gene of the invention, with a plant not comprising said modified PPO gene, non-functional HLS1 gene and non-functional BAG4 gene;

b) optionally performing one or more rounds of selfing and/or crossing a plant resulting from step a) to obtain a further generation population;

c) selecting from the population a plant that homozygously may comprise the modified PPO gene and homozygously may comprise the non-functional HLS1 gene of the invention and/or the non-functional BAG4 gene of the invention, that produces seeds that have a pale seed color and a microseed size.

The present invention also relates to a method for the production of a watermelon plant that produces seeds that have a pale seed color and a microseed size, said method which may comprise:

a) crossing a plant which may comprise the modified PPO gene of the invention with a plant which may comprise the non-functional HLS1 gene of the invention and/or the non-functional BAG4 gene of the invention;

b) optionally performing one or more rounds of selfing and/or crossing a plant resulting from step a) to obtain a further generation population;

c) selecting from the population a plant that homozygously may comprise the modified PPO gene and homozygously may comprise the non-functional HLS1 gene of the invention and/or the non-functional BAG4 gene of the invention, that produces seeds that have a pale seed color and a microseed size.

The present invention relates to a method for the production of a watermelon plant that produces seeds that have a pale seed color, said method which may comprise:

a) crossing a plant which may comprise the modified PPO gene of the invention with a plant not comprising said modified PPO gene;

b) backcrossing the plant resulting from step a) with the parent not comprising the modified PPO gene for at least three generations;

c) selecting from the third or higher backcross population a plant that homozygously may comprise the modified PPO gene that produces seeds that have a pale seed color.

The present invention also relates to a method for the production of a watermelon plant that produces seeds that have a pale seed color and a microseed size, said method which may comprise:

a) crossing a plant which may comprise the modified PPO gene of the invention and may comprise the non-functional HLS1 gene of the invention and/or the non-functional BAG4 gene of the invention, with a plant not comprising said modified PPO gene, non-functional HLS1 gene and non-functional BAG4 gene;

b) backcrossing the plant resulting from step a) with a plant not comprising said modified PPO gene, non-functional HLS1 gene and non-functional BAG4 gene for at least three generations;

c) selecting from the third or higher backcross population a plant that homozygously may comprise the modified PPO gene and homozygously may comprise the non-functional HLS1 gene of the invention and/or the non-functional BAG4 gene of the invention, that produces seeds that have a pale seed color and a microseed size.

The presence of a modified PPO gene and/or modified PPO protein leading to a pale seed color may be detected using routine methods known to the skilled person such as RT-PCR, PCR, antibody-based assays, sequencing and genotyping assays, or combinations thereof. Such methods may be used to determine for example, a reduction of the expression of the wild type PPO gene, a reduction of the expression of wild type PPO protein, the presence of a modified mRNA, cDNA or genomic DNA encoding a modified PPO protein, or the presence of a modified PPO protein, in plant material or plant parts, or DNA or RNA or protein derived therefrom. Using the same routine methods the presence of a non-functional BAG4 and/or HLS1 gene and/or modified BAG4 and/or HLS1 protein leading to a microseed size may be detected.

Modifications or mutations of the wild type PPO gene, the wild type BAG4 and/or the wild type HLS1 gene can be introduced randomly by means of one or more chemical compounds, such as ethyl methane sulphonate (EMS), nitrosomethylurea, hydroxylamine, proflavine, N-methly-N-nitrosoguanidine, N-ethyl-N-nitrosourea, N-methyl-N-nitro-nitrosoguanidine, diethyl sulphate, ethylene imine, sodium azide, formaline, urethane, phenol and ethylene oxide, and/or by physical means, such as UV-irradiation, fast neutron exposure, X-rays, gamma irradiation, and/or by insertion of genetic elements, such as transposons, T-DNA, retroviral elements.

Mutagenesis also comprises the more specific, targeted introduction of at least one modification by means of homologous recombination, oligonucleotide-based mutation introduction, zinc-finger nucleases (ZFN), transcription activator-like effector nucleases (TALENs) or Clustered Regularly Interspaced Short Palindromic Repeat (CRISPR) systems.

Modifying the wild type PPO gene, the wild type BAG4 and/or the wild type HLS1 gene could also comprise the step of targeted genome editing, wherein the sequence of the wild type PPO gene, the wild type BAG4 and/or the wild type HLS1 gene is modified, or wherein the wild type PPO gene, the wild type BAG4 and/or the wild type HLS1 gene is replaced by, respectively, another PPO, BAG4 or HLS1 gene that is modified. This can be achieved by means of any method known in the art for modifying DNA in the genome of a plant, or by means of methods for gene replacement. Such methods include genome editing techniques and homologous recombination.

Homologous recombination allows the targeted insertion of a nucleic acid construct into a genome, and the targeting is based on the presence of unique sequences that flank the targeted integration site. For example, the wild type locus of a PPO gene could be replaced by a nucleic acid construct which may comprise a modified PPO gene, the wild type locus of the BAG4 gene could be replaced by a nucleic acid construct which may comprise a modified BAG4 gene, and/or the wild type locus of the HLS1 gene could be replaced by a nucleic acid construct which may comprise a modified HLS1 gene.

Modifying the wild type PPO, the wild type BAG4 and/or the wild type HLSJ gene can involve inducing double strand breaks in DNA using zinc-finger nucleases (ZFN), TAL (transcription activator-like) effector nucleases (TALEN), Clustered Regularly Interspaced Short Palindromic Repeats/CRISPR-associated nuclease (CRISPR/Cas nuclease), or homing endonucleases that have been engineered to make double-strand breaks at specific recognition sequences in the genome of a plant, another organism, or a host cell.

TAL effector nucleases (TALENs) can be used to make double-strand breaks at specific recognition sequences in the genome of a plant for gene modification or gene replacement through homologous recombination. TAL effector nucleases are a class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, Fok I. The unique, modular TAL effector DNA binding domain allows for the design of proteins with potentially any given DNA recognition specificity. Thus, the DNA binding domains of the TAL effector nucleases can be engineered to recognise specific DNA target sites and thus, used to make double-strand breaks at desired target sequences.

ZFNs can be used to make double-strand breaks at specific recognition sequences in the genome of a plant for gene modification or gene replacement through homologous recombination. The Zinc Finger Nuclease (ZFN) is a fusion protein comprising the part of the Fok I restriction endonuclease protein responsible for DNA cleavage and a zinc finger protein which recognizes specific, designed genomic sequences and cleaves the double-stranded DNA at those sequences, thereby producing free DNA ends (Urnov et al, 2010, Nat. Rev. Genet. 11:636-46; Carroll, 2011, Genetics 188:773-82).

The CRISPR/Cas nuclease system can also be used to make double-strand breaks at specific recognition sequences in the genome of a plant for gene modification or gene replacement through homologous recombination. The CRISPR/Cas nuclease system is an RNA-guided DNA endonuclease system performing sequence-specific double-stranded breaks in a DNA segment homologous to the designed RNA. It is possible to design the specificity of the sequence (Jinek et al, 2012, Science 337: 816-821; Cho et al, 2013, Nat. Biotechnol. 31:230-232; Cong et al, 2013, Science 339:819-823; Mali et al., 2013, Science 339:823-826; Feng et al, 2013, Cell Res. 23:1229-1232). Cas9 is an RNA-guided endonuclease that has the capacity to create double-stranded breaks in DNA in vitro and in vivo, also in eukaryotic cells. It is part of an RNA-mediated adaptive defence system known as Clustered Regularly Interspaced Short Palindromic Repeats (CRISPR) in bacteria and archaea. Cas9 gets sequence-specificity when it associates with a guide RNA molecule, which can target sequences present in an organism's DNA based on their sequence. Cas9 requires the presence of a Protospacer Adjacent Motif (PAM) immediately following the DNA sequence that is targeted by the guide RNA. The Cas9 enzyme has been first isolated from Streptococcus pyogenes (SpCas9), but functional homologues from many other bacterial species have been reported, such as Neisseria meningitides, Treponema denticola, Streptococcus thermophilus, Francisella novicida, Staphylococcus aureus, etcetera. For SpCas9, the PAM sequence is 5′-NGG-3′, whereas various Cas9 proteins from other bacteria have been shown to recognise different PAM sequences. In nature, the guide RNA is a duplex between crRNA and tracrRNA, but a single guide RNA (sgRNA) molecule comprising both crRNA and tracrRNA has been shown to work equally well (Jinek et al, 2012, Science 337: 816-821). The advantage of using an sgRNA is that it reduces the complexity of the CRISPR-Cas9 system down to two components, instead of three. For use in an experimental setup (in vitro or in vivo) this is an important simplification.

An alternative for Cas9 is, for example, Cpf1, which does not need a tracrRNA to function, which recognises a different PAM sequence, and which creates sticky end cuts in the DNA, whereas Cas9 creates blunt ends.

On the one hand, genetic modification techniques can be applied to express a site-specific nuclease, such as an RNA-guided endonuclease and/or guide RNAs, in eukaryotic cells. One or more DNA constructs encoding an RNA-guided endonuclease and at least one guide RNA can be introduced into a cell or organism by means of stable transformation (wherein the DNA construct is integrated into the genome) or by means of transient expression (wherein the DNA construct is not integrated into the genome, but it expresses an RNA-guided endonuclease and at least one guide RNA in a transient manner). This approach requires the use of a transformation vector and a suitable promoter for expression in said cell or organism. Organisms into which foreign DNA has been introduced are considered to be Genetically Modified Organisms (GMOs), and the same applies to cells derived therefrom and to offspring of these organisms. In important parts of the worldwide food market, transgenic food is not allowed for human consumption, and not appreciated by the public. There is however also an alternative, “DNA-free” delivery method of CRISPR-Cas components into intact plants that does not involve the introduction of DNA constructs into the cell or organism.

For example, introducing the mRNA encoding Cas9 into a cell or organism has been described, after in vitro transcription of said mRNA from a DNA construct encoding an RNA-guided endonuclease, together with at least one guide RNA. This approach does not require the use of a transformation vector and a suitable promoter for expression in said cell or organism.

Another known approach is the in vitro assembly of ribonucleoprotein (RNP) complexes, comprising an RNA-guided endonuclease protein (for example Cas9) and at least one guide RNA, and subsequently introducing the RNP complex into a cell or organism. In plants, the use of RNPs has been demonstrated in protoplasts, for example with polyethylene glycol (PEG) transfection (Woo et al, 2015, Nat. Biotech. 33: 1162-1164). After said modification of a genomic sequence has taken place, the protoplasts or cells can be used to produce plants that harbour said modification in their genome, using any plant regeneration method known in the art (such as in vitro tissue culture).

Breaking DNA using site specific nucleases, such as, for example, those described herein above, can increase the rate of homologous recombination in the region of the breakage. Thus, coupling of such effectors as described above with nucleases enables the generation of targeted changes in genomes which include additions, deletions and other modifications.

The present invention will be further illustrated in the Examples that follow that are for illustration purposes only and are not intended to limit the invention in any way. In the description and the Examples reference is made to the below figures.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1: Phenotypic Analysis of Seed Color

In a screening of the internal Rijk Zwaan germplasm collection for watermelon plants with seeds with a light seed color, two Citrullus mucosospermus accessions (RZ907-03 and RZ907-04) were selected that produce pale colored seeds. The color of the seeds was examined visually on fully developed, mature and dry seeds. The color of fully developed, mature and dry seeds of the two selected Citrullus mucosospermus accessions could be indicated as pale, beige, light yellow, pale yellow or light khaki.

Seed color of the dried seeds of the two selected accessions was also examined using image analysis. For this photography was conducted in a standardized set-up in a darkened room using a Nikon D7000 camera with a Nikon AF-S 35 mm f/1.8G DX 35 mm lens with circular B+W polarization filter. The standardized camera set up used daylight fluorescent lamps (4×36 watts, 5400 K, CRI 98, 40 kHz) with a polarization filter. Lamp heads were angled at 45 degrees to the sample platform. Prior to taking photographs, lamps were turned on and allowed to warm up for at least 30 minutes. The camera was mounted on a stand with the lens pointing down and positioned over the sample platform. About 10 seeds that were clearly separated from each other were photographed for each sample. In each photograph an X-rite color-checker passport color-chart was included.

Color correction of the photographs was performed using an ImageJ macro (1.48u) and the X-rite color-checker passport color-chart. Image analysis and generation of calibrated RGB values was performed using a CellProfiler pipeline. The calibrated RGB color values were then translated into CIELAB L*a*b* color values (D65 illuminant and a 10 degree angle of observer) using a color calibration algorithm.

The average calibrated RGB values and calibrated CIE L*a*b* values (10°/D65) as determined for fully developed, mature and dry seeds of the selected Citrullus mucosospermus accessions with a pale seed color and two Citrullus lanatus var. lanatus accessions with dark seeds are presented in Table 2.

The L* (10° /D65) values indicates lightness on a 0 to 100 scale, where 0 is black and 100 is white. As is clear from Table 2, the L* values most clearly show the color difference between the pale and dark seeds: for the pale seeds of the two selected Citrullus mucosospermus accessions the average L*(10° /D65) values were 66.10 and 72.20; whereas the average L* (10° /D65) value was 46.50 for the brown Citrullus lanatus var. lanatus accession, and 34.70 for the black Citrullus lanatus var. lanatus accession.

TABLE 2
Average color in calibrated RGB values and in calibrated CIE L*a*b* values
(10°/D65) of fully developed, mature and dry seeds of the selected Citrullus mucosospermus
accessions with a pale seed color and two Citrullus lanatus var. lanatus accessions with dark seeds,
as determined by image analysis.

| | Average calibrated color | | | Average color dried seeds | | |
|---|---|---|---|---|---|---|
| | dried seeds | | | (10°/D65) | | |
| Accession | R | G | B | L* | a* | b* |
| RZ907-03 | 216 | 169 | 95 | 72.2 | 8.62 | 43.85 |
| RZ907-04 | 205 | 150 | 78 | 66.10 | 13.43 | 45.95 |
| RZ907-01 (black seeds) | 109 | 73 | 61 | 34.70 | 13.63 | 13.48 |
| Giong(brown seeds) | 140 | 103 | 72 | 46.50 | 11.22 | 22.42 |

### Example 2: Phenotypic Analysis of Seed Size

In a screening for watermelon plants with seeds with a small seed size two Citrullus lanatus var. lanatus accessions were selected that produce seeds with a microseed size. The size of the fully developed, mature and dry seeds was initially assessed by visual inspection and by determining the 100SDW (weight of a batch of 100 seeds). The 100SDW of Citrullus lanatus var. lanatus accession RZ907-01 was 0.8 g on average (standard deviation 0.3), while the 100SDW of Citrullus lanatus var. lanatus accession RZ907-02 was 0.6 g on average (standard deviation 0.1). The 100SDW for the seeds of the two Citrullus lanatus var. lanatus accession with microseeds and that of three control accession with medium or big seeds is presented in Table 3.

Seed size of the fully developed, mature and dry seeds of the two selected accessions and control accessions with medium or big seeds was examined by weighing with a precision balance and measuring with a caliper. For each accession 3 to 10 batches of seeds were weighed and measured, and the average 100SDW and seed sizes calculated.

The average 100SDW and seed sizes determined are presented in Table 3.

TABLE 3
Average weight of 100 seeds (100SDW) and average seed size of fully
developed mature dried seeds of the selected Citrullus lanatus var.
lanatus accessions with a microseed size and three accessions
with medium or big seeds, as determined visually, by weighing with
a precision balance and by measuring with a caliper.

| | Seed size | Average | Average seed size | | |
|---|---|---|---|---|---|
| | determined | 100SDW | Length | Width | Thickness |
| Accession | visually | (g) | (mm) | (mm) | (mm) |
| RZ907-01 | micro | 0.8 ± 0.3 | 4.0 ± 1.2 | 2.8 ± 1.0 | 1.25 ± 0.5 |
| RZ907-02 | micro | 0.6 ± 0.1 | 4.0 ± 0 | 2.4 ± 0.4 | 1.2 ± 0.4 |
| Giong | medium | 2.1 ± 0.1 | 6.1 ± 0.4 | 4.3 ± 0.5 | 2.0 ± 0 |
| RZ907-03 | medium | 5.1 ± 0.6 | 9.3 ± 0.4 | 4.7 ± 0.4 | 2.0 ± 0 |
| RZ907-04 | big | 11.4 ± 2.3 | 12.7 ± 0.4 | 7.2 ± 0.8 | 2.0 ± 0 |

### Example 3: QTL Mapping and Gene Identification for Pale Seed Color and Microseed Size

Four mapping populations were developed in order to map both the genomic region responsible for the pale seed trait and the genomic region responsible for the microseed size trait.

A first mapping population for mapping the genomic region responsible for the pale seed trait resulted from a cross between a Citrullus mucosospermus plant (RZ907-04) with pale colored seeds with a normal seed size and a Citrullus lanatus var. lanatus plant (Giong) with brown seeds of medium seed size. 182 RIL lines were developed up until the F5 generation, in order to map the genomic region responsible for the pale seed color. The color of the seeds produced by these F5 plants was phenotyped by image analysis, while the F5 plants were genotyped using 168 markers of which 104 where informative for the map construction.

Two mapping populations resulted from a cross between a Citrullus mucosospermus plant (RZ907-03) with pale colored seeds with a normal seed size and a Citrullus lanatus var. lanatus plant (RZ907-01) with dark colored microseeds. The F2 population was used for initial QTL mapping of both the pale seed color and microseed size traits. For finemapping and validation of the QTLs, near isogenic lines (NILs) were developed out of the F2 plants by using Citrullus lanatus var. lanatus plant (RZ907-01) as the recurrent backcross (BC) parent. Both the parent lines, the F2 population (155 individuals genotyped with 137 informative markers) and the BC3F3 NILs population (182 informative individuals genotyped with 136 informative markers) were phenotyped for seed color and seed size and genotyped for genetic map construction.

For finemapping and validation of the QTLs a pale seeded NIL (BC3F2) from the cross between a Citrullus mucosospermus plant (RZ907-04) and a Citrullus lanatus var. lanatus plant (Giong), was crossed with a Citrullus lanatus var. lanatus plant (RZ907-02) with dark colored microseeds. All three parent lines and the F2 population from this cross (181 individuals genotyped with 104 informative markers) were phenotyped for seed color and seed size and genotyped for genetic map construction.

In the development of these mapping populations both the pale seed color trait and the microseed size trait showed a monogenic recessive inheritance. It also became clear that both the pale seed color and the microseed size phenotype are determined by the genotype of the plant that produces the seed.

Before the genetic map construction, non-polymorphic and uninformative markers and strong deviant individuals were removed. During the genetic map construction two linkage groups were constructed. Numbering and orientation of the linkage groups was done using physical assembly cl_97103_v1 as reference, publicly available at http://cucurbitgenomics.org. Marker phase correction was performed using the marker information from the parental lines and the grouping structure. Map integration was performed using the physical assembly cl_97103_v1 as reference. QTL analysis was performed using the R software package.

A quantitative trait locus (QTL) for pale seed color on Chromosome 3 was identified in all 4 mapping populations. By fine mapping the QTL region could be reduced to a region of approximately 110 kB, which comprises 5 PPO genes and one unknown predicted gene.

By studying whole genome sequence data of both sources of the pale seed color trait and comparing the sequence with whole genome sequence data of material with dark colored seeds it was clear that there is a C/CA indel at position cl_97103_v1_Chr3:5706705 in the sources with pale colored seeds. The insertion of an A at position cl_97103_v1_Chr3:5706705 leads to a frameshift, which leads to the introduction of a premature stop codon in the PPO gene of SEQ ID NO: 1 (which gene is in the genome on the reverse strand). The modified PPO gene comprises an insertion of a T between nucleotides 711 and 712 of SEQ ID NO: 1. This one base pair insertion leads to a frameshift, which leads to 13 amino acids being encoded in the wrong frame followed by a premature stop codon at position 751-753 of the modified PPO gene (SEQ ID NO:2).

Whereas the size of the wild type PPO protein is 587 amino acids (SEQ ID NO:5), the modified PPO protein (SEQ ID NO:6), if produced at all, is only 250 amino acids long, comprises only a small part of its Tyrosinase domain, lacks its conserved PPO1-DWL and PPO1-KFDV domains completely and comprises 13 altered amino acids at its C-terminus. The mutant protein is thus not functional.

A marker (named CL08381) was designed on the C/CA indel at position cl_97103_v1_Chr3:5706705. SEQ ID NO: 15 represents the allele of marker CL08381 as it is present in the genome of a plant which may comprise the modified PPO gene of this invention and producing pale colored seeds. SEQ ID NO:14 represents the wild type allele of this same marker, as is present in genomes of plants that do not comprise the modified PPO gene of this invention. The nucleotide that is different between the two marker alleles of marker CL08381 is underlined and in bold in Table 4 below. The marker allele (SEQ ID NO: 15) for the modified PPO gene has a single nucleotide insertion of an A that is underlined and in bold in Table 2 (position 101 of SEQ ID NO:15). In all four mapping populations this marker showed a 100% correlation with the pale seed phenotype.

A quantitative trait locus (QTL) for microseed size on Chromosome 2 was identified in 3 mapping populations. In two of these populations the QTL interval was only 1 cM. By studying whole genome sequence data of both sources of the microseed size trait and comparing the sequence with whole genome sequence data of material with big or medium sized seeds it was clear that only in material with a microseed size (about 30 kB from the peak markers of two of the QTL populations) there is genomic deletion, corresponding to 13962 bp being deleted between base pair position 29902114 and 29916077 on chromosome 2 of the Citrullus lanatus 97103_v1 genome. In other words, all nucleotides starting from the G at position 29902115 on chromosome 2 of the Citrullus lanatus 97103_v1 genome to the A at position 29916076 on chromosome 2 of the Citrullus lanatus 97103_v1 genome, have been deleted. Sequence SEQ ID NO:13 provides the cl_97103_v1 genomic sequence from position 29897185 to 29920517 of chromosome 2. The genomic deletion conferring microseed size corresponds to a deletion of all nucleotides between base pair position 4930 and 18893 of SEQ ID NO: 13.

This genomic deletion leads to two genes being deleted: the HLS1 gene of SEQ ID NO: 7 and the BAG4 gene of SEQ ID NO: 10.

Markers were designed to detect the presence or the absence of the genomic deletion on chromosome 2 (CL_chr2_gap1 and CL_chr2_gap2 as included in Table 4 below): In material comprising the genome deletion the primers of marker CL_chr2_gap1 (SEQ ID NO:16 and 17) amplify a PCR product of 446 bp, in material without said genome deletion no PCR product is amplified by these primers. In material not comprising said genome deletion the primers of marker CL_chr2_gap2 (SEQ ID NO:18 and 19) amplify a PCR product of 945 bp, in material comprising said genome deletion no PCR product is amplified by these primers.

A PCR on DNA isolated from a plant producing microseeds gave a PCR product of 446 bp in a PCR with the primers of marker CL_chr2_gap1 (SEQ ID NO:16 and 17), and no PCR product in a PCR with the primers of marker CL_chr2_gap2 (SEQ ID NO:18 and 19). A PCR on DNA isolated from a plant producing big seeds gave no PCR product in a PCR with the primers of marker CL_chr2_gap1 (SEQ ID NO:16 and 17), and a PCR product of 945 bp in a PCR with the primers of marker CL_chr2_gap2 (SEQ ID NO:18 and 19). A PCR on DNA isolated from an F1 plant resulting from a cross between a plant producing big seeds and a plant producing microseeds gave a PCR product of 446 bp in a PCR with the primers of marker CL_chr2_gap 1 (SEQ ID NO:16 and 17), and a PCR product of 945 bp in a PCR with the primers of marker CL_chr2_gap2 (SEQ ID NO:18 and 19).

TABLE 4
Marker information.

| Marker name | Seq ID NO: | Sequence marker |
|---|---|---|
| CL08381 | SEQ ID NO: 14 | MACCAATGTCGGCGGCT |
| (pale seed | (wild type | GATGACGTCCGTCACGA |
| color) | allele) | AATGCATCATARAGTGA |
| | | CGAACTTTTATCCGTGT |
| | | AGATTTTTGGTATTTCC |
| | | ATCCCTTGCGGAGCCGT |
| | | CATAATTCCAAAACGGC |
| | | AATGCAAAATCAGGATC |
| | | CTTAATCAAAGACCCCA |
| | | ATATTCTCTCATGAAAG |
| | | TAAAGATAAAAACGATG |
| | | GAATGGGAAGAAC |
| | SEQ ID NO: 15 | MACCAATGTCGGCGGCT |
| | (mutant | GATGACGTCCGTCACGA |
| | allele) | AATGCATCATARAGTGA |
| | | CGAACTTTTATCCGTGT |
| | | AGATTTTTGGTATTTCC |
| | | ATCCCTTGCGGAGCCAG |
| | | TCATAATTCCAAAACGG |
| | | CAATGCAAAATCAGGAT |
| | | CCTTAATCAAAGACCCC |
| | | AATATTCTCTCATGAAA |
| | | GTAAAGATAAAAACGAT |
| | | GGAATGGGAAGAAC |
| CL_chr2_gap1 | SEQ ID NO: 16 | AGAGTGAACCAAAAGAT |
| (microseed | (primer F1) | CC |
| size) | SEQ ID NO: 17 | CCCAAAACCAAATAGTT |
| | (primer R3) | ACC |
| CL_chr2_gap2 | SEQ ID NO: 18 | GAACCAAAAGATCCACC |
| (microseed | (primer F2) | A |
| size | SEQ ID NO: 19 | ACCTACATCCACTCCCT |
| | (primer R1) | AA |

### Example 4: Combining the Pale Seed Color and Microseed Size Traits in One Plant

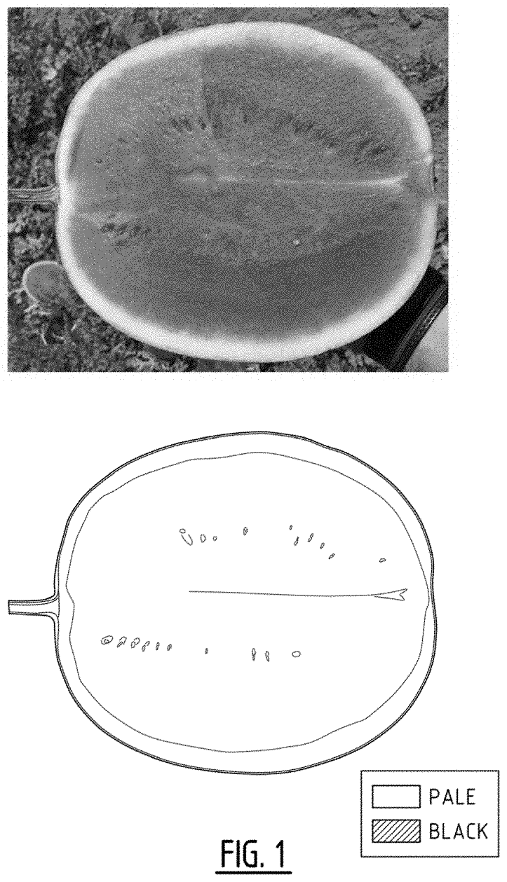

Out of the cross between a Citrullus mucosospermus plant (RZ907-03) with pale colored seeds with a normal seed size and a Citrullus lanatus var. lanatus plant (RZ907-01) with dark colored microseeds, plants were selected using markers SEQ ID NO: 14-19 in which both the microseed and the pale seed trait were fixed. Besides selecting for these two traits plants were also selected for having fruit flesh that is red and has a high brix level. These selected plants were homozygous for both the modified PPO gene and the deletion of 13962 bp on Chromosome 2 corresponding to base pair position 29902114 and 29916077 on the Citrullus lanatus 97103_v1 genome. FIG. 1 shows a picture of a mature fruit of such a plant. The mature fruits of the selected plants have red fruit flesh with pale colored microseeds. The brix levels of mature fruits of these plants vary between 9.0 degrees Brix and 14.4 degrees Brix. Seeds resulting from a self-pollination on such a plant with an average brix level of mature fruits of 10.0 (Std 0.87) were deposited at the NCIMB under deposit Accession number 43364. Table 5 and Table 6 present the color and seed size data gathered on fully developed, mature and dried seeds as deposited and seeds of control varieties with big or medium sized seeds and/or seeds with a dark color.

TABLE 5
Average seed color of fully developed mature dried seeds in calibrated RGB
values and in calibrated CIE L*a*b* values (10°/D65) for seeds of the deposit NCIMB 43364 with
a pale seed color and microseed size and for brown (variety Giong) and black seeds (RZ907-1) not
comprising the modified PPO gene of the invention, as determined by image analysis. Photographs
were taken on a black background.

| | Average calibrated | | | Average color dried | | | Standard deviation | | |
|---|---|---|---|---|---|---|---|---|---|
| | color dried seeds | | | seeds (10°/D65) | | | between seeds | | |
| Accession | R | G | B | L* | a* | b* | L* Std | a* Std | b* Std |
| Deposit | 194.5 | 154.8 | 103.9 | 66.5 | 8.5 | 31.7 | 2.5 | 0.5 | 0.1 |
| NCIMB 43364 | | | | | | | | | |
| Giong (brown | 139.8 | 102.6 | 72.4 | 46.5 | 11.2 | 22.4 | 6.0 | 0.3 | 2.8 |
| seeds) | | | | | | | | | |
| RZ907-01 | 109.0 | 73.2 | 60.7 | 34.7 | 13.6 | 13.5 | 6.4 | 0.1 | 4.5 |
| (black seeds) | | | | | | | | | |

TABLE 6
Average seed size of fully developed mature dried seeds for seeds of the
deposit NCIMB 43364 with a pale seed color and microseed size and for medium
(variety Giong) and big (RZ907-04) sized seeds not comprising the genomic
deletion on chromosome 2 of this invention, as determined visually, by
weighing with a precision balance and by measuring with a caliper.

| | Seed size | Average | Average seed size | | |
|---|---|---|---|---|---|
| | determined | 100SDW | Length | Width | Thickness |
| Accession | visually | (g) | (mm) | (mm) | (mm) |
| Deposit NCIMB | micro | 0.7 ± 0 | 4.0 ± 0 | 2.5 ± 0.7 | 1.5 ± 0 |
| 43364 | | | | | |
| Giong | medium | 2.1 ± 0.1 | 6.1 ± 0.4 | 4.3 ± 0.5 | 2.0 ± 0 |
| RZ907-04 | big | 11.4 ± 2.3 | 12.7 ± 0.4 | 7.2 ± 0.8 | 2.0 ± 0 |

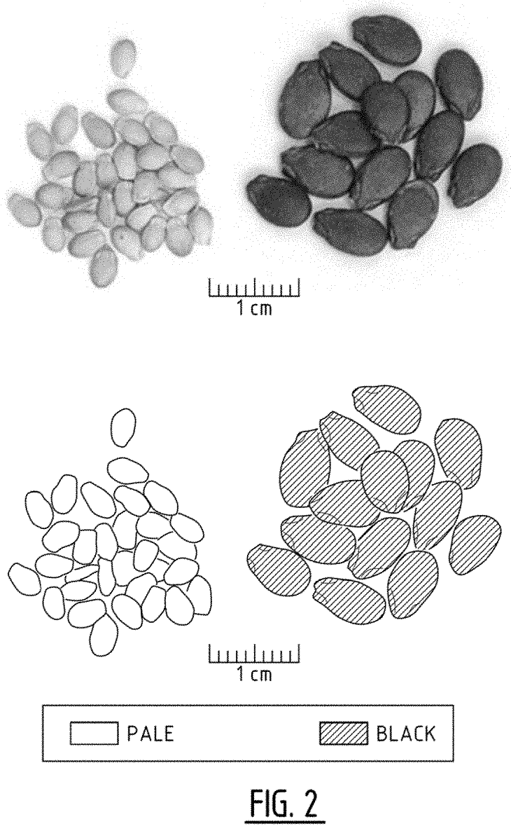

FIG. 2 shows seeds as deposited at the NCIMB under deposit Accession number 43364 with a pale color and a microseed size (left), and seeds of a wild type publicly available watermelon variety that are black and have a big seed size (right). The bar indicates a size of 1 cm.

### Example 5: Modifying the PPO Gene to Produce the Pale Seed Color Trait

Seeds of the watermelon plants of interest with dark colored seeds are mutagenized in order to introduce mutations into the genome. Mutagenesis is achieved using chemical means, such as EMS treatment, fast neutron (FN) radiation or specific targeted means such as CRISPR. The skilled person is familiar with chemical, radiation and targeted means for introducing mutations into a genome.

Mutagenized seed is then germinated, the resultant plants are selfed or crossed to produce M2 seed. A tilling screen for PPO gene modifications which are responsible for the pale seed color trait is performed. PPO gene modifications are identified based on comparison to the wild type PPO DNA sequences listed in SEQ ID NO: 1 and SEQ ID NO: 3. The skilled person is also familiar with tilling (McCallum et. al. (2000) Nature Biotechnology, 18: 455-457) and techniques for identifying nucleotide changes such as DNA sequencing, amongst others.

Watermelon plants with a modified PPO gene can be identified and selected on the basis of modifications to the PPO gene. Preferably, PPO gene knockout mutants (encoding a premature stop codon) are selected, but also PPO amino acid change mutants can result in a pale seed color. Amino acid change mutants that are most likely to be deleterious to the function of the protein and thus most likely to result in a pale seed color can be selected using a predictive tool such as SIFT or PROVEAN. Mutants are homozygous or made homozygous by selfing, crossing or doubled haploid techniques which are familiar to the skilled person. Seed color of said homozygous plants can then be analyzed visually, with a colorimeter or using image analysis, to confirm that they have a pale seed color.

### Example 6: Modifying the HLS1 Gene and/or the BAG4 Gene to Produce the Microseed Trait

Seeds of the watermelon plants of interest with medium or big seeds are mutagenized in order to introduce mutations into the genome. Mutagenesis is achieved using chemical means, such as EMS treatment, fast neutron (FN) radiation or specific targeted means such as CRISPR. The skilled person is familiar with chemical, radiation and targeted means for introducing mutations into a genome.

Mutagenized seed is then germinated, the resultant plants are selfed or crossed to produce M2 seed. A tilling screen for BAG4 gene and/or HLS1 gene modifications which are responsible for the microseed size trait is performed. HLS1 gene modifications are identified based on comparison to the wild type HLS1 DNA sequences listed in SEQ ID NO: 7 and SEQ ID NO: 8. BAG4 gene modifications are identified based on comparison to the wild type BAG4 DNA sequences listed in SEQ ID NO: 10 and SEQ ID NO: 11. The skilled person is also familiar with tilling (McCallum et. al. (2000) Nature Biotechnology, 18: 455-457) and techniques for identifying nucleotide changes such as DNA sequencing, amongst others.

Watermelon plants with a non-functional HLS1 gene can be identified and selected on the basis of deleterious mutations to the HLS1 gene. Preferably, HLS1 gene knockout mutants (encoding a premature stop codon) are selected, but also HLS1 amino acid change mutants can result in a pale seed color. Amino acid change mutants that are most likely to be deleterious to the function of the protein and thus to result in a microseed size can be selected using a tool such as SIFT or PROVEAN.

Watermelon plants with a non-functional BAG4 gene can be identified and selected on the basis of deleterious mutations to the BAG4 gene. Preferably, BAG4 gene knockout mutants (encoding a premature stop codon) are selected, but also BAG4 amino acid change mutants can result in a pale seed color. Amino acid change mutants that are most likely to be deleterious to the function of the protein and thus to result in a microseed size can be selected using a tool such as SIFT or PROVEAN. Mutants are homozygous or made homozygous by selfing, crossing or doubled haploid techniques which are familiar to the skilled person. Seed size of said homozygous plants can then be analyzed visually by measuring or by using image analysis, to confirm that the microseed size results from one or more modification to the HLS1 gene and/or BAG4 gene.

### SEQUENCE INFORMATION

TABLE 7
PPO, HLS1 and BAG4 gene and protein sequences
and their corresponding SEQ ID NOS (“CDS”: Coding
sequence).

| | gDNA | CDS DNA | Protein |
|---|---|---|---|
| Sequence Name | SEQ ID NO: | SEQ ID NO: | SEQ ID NO: |
| PPO wild type sequence | 1 | 3 | 5 |
| PPO modified sequence | 2 | 4 | 6 |
| HLS1 wild type sequence | 7 | 8 | 9 |
| BAG4 wild type sequence | 10 | 11 | 12 |
| wild type sequence | 13 | | |
| Chr2:29897185-29920517 of | | | |
| watermelon reference | | | |
| genome cl_97103_ v1 | | | |
| (available at | | | |
| http://cucurbitgenomics.org) | | | |

> PPO_WT_gDNA
SEQ ID NO: 1
ATGGCCTCTCTATCTCCTTCCATGCCACTAGCACTTTCCTCCGCCGCAATA
ACCACGGCCACCACCGGCGGCGCCTCCTTTGGTCTGTTTTATCGTAAAAAAAAAGAT
CCATCTTCCACCATTCATAGACTCAATAACTTGGTTGTGTGTAGCGGCTCCAATGGC
AGTGGTGAAGAAAGTAATAACTCATTATGGCCAGGCAAGTTTGTTGACCGGAGAGA
AGCGCTTATCGGGCTCGGCGGTCTGTATGGCTCAGCTTCAAGTGCTTTTGGAGTTGA
TCCCTTCGCTTTGGCAGCTCCAGTCACAACCCCCGACCCTTCCAAGTGTGGATCAAG
CACGGACTTGGCAGATGGCGTCAAAGATTTGGTTTGCTGCCCACCATCCACCAATAA
CGTAAAACCCTTCCTCAAACCACGCGTTAGGAAAGCGGCACAATCATTAGATAAAG
AATATATTGAAAAGTATAAGGAAGCCGTAGCGCTTATGAAAGCGCTTCCTGATGAT
GATCCACGTAGTTTTAAACAGCAAGCACTTGTTCACTGTGCTTATTGTACTGGGGGT
TACGATCAATTGGGTCTTCCAGTTGAATTACAAGTTCATTTCTCGTGGCTGTTCTTCC
CATTCCATCGTTTTTATCTTTACTTTCATGAGAGAATATTGGGGTCTTTGATTAAGGA
TCCTGATTTTGCATTGCCGTTTTGGAATTATGACGCTCCGCAAGGGATGGAAATACC
AAAAATCTACACGGATAAAAGTTCGTCACTCTATGATGCATTTCGTGACGGACGTCA
TCAGCCGCCGACATTGGTTGATTTGGATTACAATGACGTTGAGCCAACAATAAGCAG
AGAAAAGATAATCCAATGCAATCTAAGTGTTATGTATCGCCAGGTCGTGTCCGGCGC
CCGTACGCCCTTGCTCTTTTTCGGCCAGCCTTATCGAAGTGGCAGCAACCCAAGTCC
AGGTGATTTTTTTTTTCAACCATATAATTTCTTTTTTCAAAAAAAGGAAAAGATAAA
AAAGACAATCATATATAGTTATGGTTATGTTAGGTAATGTTTTTAGTTTTTGAAAATT
AATGTTAAGAGTGTTTTTGGGTTGGATTAGAGCAGTTTATAGATCTAACTCAATTGT
CGGATTGATAAGTTTTTTCAATTCAAATAAATTCTATTAAATAATGAATCGAATTCA
ACTCAATCATGAAATATATGGGTTCAAATTACTTAGGTCATTTATGTAAATTTCTGTT
CTAAAAATAAGTAAAATGTTAGGAAACTATTATAAATGGAAAAAAATCAAAGTTTT
TACAAATACAGAAAGATTTAATTGAGTCTATCCCCTAATGATAAAAGTTTATCGCGG
TTTATCACGGATAGACAATGAAATATTTGCAAATAATTTGACTTTTTTTGCTATATTT
GAAAACAACCCTAAAATGTTAATATGTAAAATTTTTGTTGAATTATTTTTATATATTG
AATTAAAATTAACAACTCAATTTCAATTTATATTATGAAGATTTTCTTTTCAAAATGC
TAAAATATTATTGTTTGAAGTTTCTGGAGAATAAACTTTCCAAAAAATTATGAAATT
AAATAAAATTAAAATAAATATATGTATATATAATTGTACCATAAGTAAATCTTTTAT
AAAAGAATTATAACAAGTTGGGGTTATTTGAAGAGAACACATAAACAACTCAACCA
ATAACTTTTTAATTTATTTGAACTCAACCTAACCCAATCGAAACAGTGATGATTGGG
TTGTGTTGATAAGTTATGCTGGTCATCAAATTGACTTGTTTTTTTAATTTAATCATTTG
AAAATGTATTCTAAACATGCCCATTTGTAAATTAGATAATGATGTTTAGAGATAATA
ATTAAGGATATTAGTATGGAAAGTAAAATGATGTAATTTTGATGAATTGATTAATTT
TTTGAAAGGAATGGGGACGGTGGAAAACCTTCCTCACAATTCAATTCATTTGTGGAC
GGGTGACCCGAACCAATCGAACCGAATTGACATGGGAACCTTCTTCTCAGCGGCTA
GAGATCCCATCTTCTACGCCCACCACGCGAACGTGGACCGTTTTTGGTCCATATGGA
AATCCTTAGGCGAAAAGCGACAAGACATTAAAGACAAAGATTTCCTAAACGCTTCC
TTTGTATTCTACGATGAGAATGGTGAAGCTGTCCGAGTTTATGTCAAAGACTGTCTA
GATACCAGAGCCTTAGGCTATGTCTACGATGACACCGTACCAATTCCATGGCTCAAA
ACACCTCCAACCCCACGAGTACCACGCACACCCAACAAAACCAAGAAGAAATCTAC
CAAGAAGACCGGGAAGCTACCGTCGAGTGTTGACAAGATCGTCAGCTTCGAAGTCA
AGAGGCCGAAGAAATCGAGGGGTACGAAGGAGAAAGACGATGAAGAGGAGATTTT
GGTGATTGATGGGATTGAGTTCGACGGAAACAAGGCTATTAAGTTTGATGTTTTTAT
CAATGACGAGGATGATAGGGAAATTAGAGCGGATAATTCTGAGTTTGCAGGGAGCT
TTGTGAATGTGCCTCATATGAAAGGTAGCAGCAGCATGAACATAAAAACATGCCTT
AGGTTAGGGATAACTGAACTGCTTGAGAGTTTGGATGCGGATAACGACGATAGCAT
TATTGTTACATTGGTCCCTAGGTTTGGAGATGGGTCCGCCACCGTTAAGGACATTAG
AATTGAATATGATGCATAA
> PPO_MUT_gDNA_with INDEL
SEQ ID NO: 2
ATGGCCTCTCTATCTCCTTCCATGCCACTAGCACTTTCCTCCGCCGCAATA
ACCACGGCCACCACCGGCGGCGCCTCCTTTGGTCTGTTTTATCGTAAAAAAAAAGAT
CCATCTTCCACCATTCATAGACTCAATAACTTGGTTGTGTGTAGCGGCTCCAATGGC
AGTGGTGAAGAAAGTAATAACTCATTATGGCCAGGCAAGTTTGTTGACCGGAGAGA
AGCGCTTATCGGGCTCGGCGGTCTGTATGGCTCAGCTTCAAGTGCTTTTGGAGTTGA
TCCCTTCGCTTTGGCAGCTCCAGTCACAACCCCCGACCCTTCCAAGTGTGGATCAAG
CACGGACTTGGCAGATGGCGTCAAAGATTTGGTTTGCTGCCCACCATCCACCAATAA
CGTAAAACCCTTCCTCAAACCACGCGTTAGGAAAGCGGCACAATCATTAGATAAAG
AATATATTGAAAAGTATAAGGAAGCCGTAGCGCTTATGAAAGCGCTTCCTGATGAT
GATCCACGTAGTTTTAAACAGCAAGCACTTGTTCACTGTGCTTATTGTACTGGGGGT
TACGATCAATTGGGTCTTCCAGTTGAATTACAAGTTCATTTCTCGTGGCTGTTCTTCC
CATTCCATCGTTTTTATCTTTACTTTCATGAGAGAATATTGGGGTCTTTGATTAAGGA
TCCTGATTTTGCATTGCCGTTTTGGAATTATGAC [symbol] GCTCCGCAAGGGATGGAAATAC
CAAAAATCTACACGGATAAAAGTTCGTCACTCTATGATGCATTTCGTGACGGACGTC
ATCAGCCGCCGACATTGGTTGATTTGGATTACAATGACGTTGAGCCAACAATAAGCA
GAGAAAAGATAATCCAATGCAATCTAAGTGTTATGTATCGCCAGGTCGTGTCCGGCG
CCCGTACGCCCTTGCTCTTTTTCGGCCAGCCTTATCGAAGTGGCAGCAACCCAAGTC
CAGGTGATTTTTTTTTTCAACCATATAATTTCTTTTTTCAAAAAAAGGAAAAGATAAA
AAAGACAATCATATATAGTTATGGTTATGTTAGGTAATGTTTTTAGTTTTTGAAAATT
AATGTTAAGAGTGTTTTTGGGTTGGATTAGAGCAGTTTATAGATCTAACTCAATTGT
CGGATTGATAAGTTTTTTCAATTCAAATAAATTCTATTAAATAATGAATCGAATTCA
ACTCAATCATGAAATATATGGGTTCAAATTACTTAGGTCATTTATGTAAATTTCTGTT
CTAAAAATAAGTAAAATGTTAGGAAACTATTATAAATGGAAAAAAATCAAAGTTTT
TACAAATACAGAAAGATTTAATTGAGTCTATCCCCTAATGATAAAAGTTTATCGCGG
TTTATCACGGATAGACAATGAAATATTTGCAAATAATTTGACTTTTTTTGCTATATTT
GAAAACAACCCTAAAATGTTAATATGTAAAATTTTTGTTGAATTATTTTTATATATTG
AATTAAAATTAACAACTCAATTTCAATTTATATTATGAAGATTTTCTTTTCAAAATGC
TAAAATATTATTGTTTGAAGTTTCTGGAGAATAAACTTTCCAAAAAATTATGAAATT
AAATAAAATTAAAATAAATATATGTATATATAATTGTACCATAAGTAAATCTTTTAT
AAAAGAATTATAACAAGTTGGGGTTATTTGAAGAGAACACATAAACAACTCAACCA
ATAACTTTTTAATTTATTTGAACTCAACCTAACCCAATCGAAACAGTGATGATTGGG
TTGTGTTGATAAGTTATGCTGGTCATCAAATTGACTTGTTTTTTTAATTTAATCATTTG
AAAATGTATTCTAAACATGCCCATTTGTAAATTAGATAATGATGTTTAGAGATAATA
ATTAAGGATATTAGTATGGAAAGTAAAATGATGTAATTTTGATGAATTGATTAATTT
TTTGAAAGGAATGGGGACGGTGGAAAACCTTCCTCACAATTCAATTCATTTGTGGAC
GGGTGACCCGAACCAATCGAACCGAATTGACATGGGAACCTTCTTCTCAGCGGCTA
GAGATCCCATCTTCTACGCCCACCACGCGAACGTGGACCGTTTTTGGTCCATATGGA
AATCCTTAGGCGAAAAGCGACAAGACATTAAAGACAAAGATTTCCTAAACGCTTCC
TTTGTATTCTACGATGAGAATGGTGAAGCTGTCCGAGTTTATGTCAAAGACTGTCTA
GATACCAGAGCCTTAGGCTATGTCTACGATGACACCGTACCAATTCCATGGCTCAAA
ACACCTCCAACCCCACGAGTACCACGCACACCCAACAAAACCAAGAAGAAATCTAC
CAAGAAGACCGGGAAGCTACCGTCGAGTGTTGACAAGATCGTCAGCTTCGAAGTCA
AGAGGCCGAAGAAATCGAGGGGTACGAAGGAGAAAGACGATGAAGAGGAGATTTT
GGTGATTGATGGGATTGAGTTCGACGGAAACAAGGCTATTAAGTTTGATGTTTTTAT
CAATGACGAGGATGATAGGGAAATTAGAGCGGATAATTCTGAGTTTGCAGGGAGCT
TTGTGAATGTGCCTCATATGAAAGGTAGCAGCAGCATGAACATAAAAACATGCCTT
AGGTTAGGGATAACTGAACTGCTTGAGAGTTTGGATGCGGATAACGACGATAGCAT
TATTGTTACATTGGTCCCTAGGTTTGGAGATGGGTCCGCCACCGTTAAGGACATTAG
AATTGAATATGATGCATAA
> PPO_WT_CDS
SEQ ID NO: 3
ATGGCCTCTCTATCTCCTTCCATGCCACTAGCACTTTCCTCCGCCGCAATA
ACCACGGCCACCACCGGCGGCGCCTCCTTTGGTCTGTTTTATCGTAAAAAAAAAGAT
CCATCTTCCACCATTCATAGACTCAATAACTTGGTTGTGTGTAGCGGCTCCAATGGC
AGTGGTGAAGAAAGTAATAACTCATTATGGCCAGGCAAGTTTGTTGACCGGAGAGA
AGCGCTTATCGGGCTCGGCGGTCTGTATGGCTCAGCTTCAAGTGCTTTTGGAGTTGA
TCCCTTCGCTTTGGCAGCTCCAGTCACAACCCCCGACCCTTCCAAGTGTGGATCAAG
CACGGACTTGGCAGATGGCGTCAAAGATTTGGTTTGCTGCCCACCATCCACCAATAA
CGTAAAACCCTTCCTCAAACCACGCGTTAGGAAAGCGGCACAATCATTAGATAAAG
AATATATTGAAAAGTATAAGGAAGCCGTAGCGCTTATGAAAGCGCTTCCTGATGAT
GATCCACGTAGTTTTAAACAGCAAGCACTTGTTCACTGTGCTTATTGTACTGGGGGT
TACGATCAATTGGGTCTTCCAGTTGAATTACAAGTTCATTTCTCGTGGCTGTTCTTCC
CATTCCATCGTTTTTATCTTTACTTTCATGAGAGAATATTGGGGTCTTTGATTAAGGA
TCCTGATTTTGCATTGCCGTTTTGGAATTATGACGCTCCGCAAGGGATGGAAATACC
AAAAATCTACACGGATAAAAGTTCGTCACTCTATGATGCATTTCGTGACGGACGTCA
TCAGCCGCCGACATTGGTTGATTTGGATTACAATGACGTTGAGCCAACAATAAGCAG
AGAAAAGATAATCCAATGCAATCTAAGTGTTATGTATCGCCAGGTCGTGTCCGGCGC
CCGTACGCCCTTGCTCTTTTTCGGCCAGCCTTATCGAAGTGGCAGCAACCCAAGTCC
AGGAATGGGGACGGTGGAAAACCTTCCTCACAATTCAATTCATTTGTGGACGGGTG
ACCCGAACCAATCGAACCGAATTGACATGGGAACCTTCTTCTCAGCGGCTAGAGAT
CCCATCTTCTACGCCCACCACGCGAACGTGGACCGTTTTTGGTCCATATGGAAATCC
TTAGGCGAAAAGCGACAAGACATTAAAGACAAAGATTTCCTAAACGCTTCCTTTGTA
TTCTACGATGAGAATGGTGAAGCTGTCCGAGTTTATGTCAAAGACTGTCTAGATACC
AGAGCCTTAGGCTATGTCTACGATGACACCGTACCAATTCCATGGCTCAAAACACCT
CCAACCCCACGAGTACCACGCACACCCAACAAAACCAAGAAGAAATCTACCAAGAA
GACCGGGAAGCTACCGTCGAGTGTTGACAAGATCGTCAGCTTCGAAGTCAAGAGGC
CGAAGAAATCGAGGGGTACGAAGGAGAAAGACGATGAAGAGGAGATTTTGGTGAT
TGATGGGATTGAGTTCGACGGAAACAAGGCTATTAAGTTTGATGTTTTTATCAATGA
CGAGGATGATAGGGAAATTAGAGCGGATAATTCTGAGTTTGCAGGGAGCTTTGTGA
ATGTGCCTCATATGAAAGGTAGCAGCAGCATGAACATAAAAACATGCCTTAGGTTA
GGGATAACTGAACTGCTTGAGAGTTTGGATGCGGATAACGACGATAGCATTATTGTT
ACATTGGTCCCTAGGTTTGGAGATGGGTCCGCCACCGTTAAGGACATTAGAATTGAA
TATGATGCATAA
>PPO_MUT_CDS
SEQ ID NO: 4
ATGGCCTCTCTATCTCCTTCCATGCCACTAGCACTTTCCTCCGCCGCAATA
ACCACGGCCACCACCGGCGGCGCCTCCTTTGGTCTGTTTTATCGTAAAAAAAAAGAT
CCATCTTCCACCATTCATAGACTCAATAACTTGGTTGTGTGTAGCGGCTCCAATGGC
AGTGGTGAAGAAAGTAATAACTCATTATGGCCAGGCAAGTTTGTTGACCGGAGAGA
AGCGCTTATCGGGCTCGGCGGTCTGTATGGCTCAGCTTCAAGTGCTTTTGGAGTTGA
TCCCTTCGCTTTGGCAGCTCCAGTCACAACCCCCGACCCTTCCAAGTGTGGATCAAG
CACGGACTTGGCAGATGGCGTCAAAGATTTGGTTTGCTGCCCACCATCCACCAATAA
CGTAAAACCCTTCCTCAAACCACGCGTTAGGAAAGCGGCACAATCATTAGATAAAG
AATATATTGAAAAGTATAAGGAAGCCGTAGCGCTTATGAAAGCGCTTCCTGATGAT
GATCCACGTAGTTTTAAACAGCAAGCACTTGTTCACTGTGCTTATTGTACTGGGGGT
TACGATCAATTGGGTCTTCCAGTTGAATTACAAGTTCATTTCTCGTGGCTGTTCTTCC
CATTCCATCGTTTTTATCTTTACTTTCATGAGAGAATATTGGGGTCTTTGATTAAGGA
TCCTGATTTTGCATTGCCGTTTTGGAATTATGAC [symbol] GCTCCGCAAGGGATGGAAATAC
CAAAAATCTACACGGATAAAAGTTCGTCACTCTATGATGCATTTCGTGACGGACGTC
ATCAGCCGCCGACATTGGTTGATTTGGATTACAATGACGTTGAGCCAACAATAAGCA
GAGAAAAGATAATCCAATGCAATCTAAGTGTTATGTATCGCCAGGTCGTGTCCGGCG
CCCGTACGCCCTTGCTCTTTTTCGGCCAGCCTTATCGAAGTGGCAGCAACCCAAGTC
CAGGAATGGGGACGGTGGAAAACCTTCCTCACAATTCAATTCATTTGTGGACGGGTG
ACCCGAACCAATCGAACCGAATTGACATGGGAACCTTCTTCTCAGCGGCTAGAGAT
CCCATCTTCTACGCCCACCACGCGAACGTGGACCGTTTTTGGTCCATATGGAAATCC
TTAGGCGAAAAGCGACAAGACATTAAAGACAAAGATTTCCTAAACGCTTCCTTTGTA
TTCTACGATGAGAATGGTGAAGCTGTCCGAGTTTATGTCAAAGACTGTCTAGATACC
AGAGCCTTAGGCTATGTCTACGATGACACCGTACCAATTCCATGGCTCAAAACACCT
CCAACCCCACGAGTACCACGCACACCCAACAAAACCAAGAAGAAATCTACCAAGAA
GACCGGGAAGCTACCGTCGAGTGTTGACAAGATCGTCAGCTTCGAAGTCAAGAGGC
CGAAGAAATCGAGGGGTACGAAGGAGAAAGACGATGAAGAGGAGATTTTGGTGAT
TGATGGGATTGAGTTCGACGGAAACAAGGCTATTAAGTTTGATGTTTTTATCAATGA
CGAGGATGATAGGGAAATTAGAGCGGATAATTCTGAGTTTGCAGGGAGCTTTGTGA
ATGTGCCTCATATGAAAGGTAGCAGCAGCATGAACATAAAAACATGCCTTAGGTTA
GGGATAACTGAACTGCTTGAGAGTTTGGATGCGGATAACGACGATAGCATTATTGTT
ACATTGGTCCCTAGGTTTGGAGATGGGTCCGCCACCGTTAAGGACATTAGAATTGAA
TATGATGCATAA
> PPO_WT_protein
SEQ ID NO: 5
MASLSPSMPLALSSAAITTATTGGASFGLFYRKKKDPSSTIHRLNNLVVCSGS
NGSGEESNNSLWPGKFVDRREALIGLGGLYGSASSAFGVDPFALAAPVTTPDPSKCGSST
DLADGVKDLVCCPPSTNNVKPFLKPRVRKAAQSLDKEYIEKYKEAVALMKALPDDDPR
SFKQQALVHCAYCTGGYDQLGLPVELQVHFSWLFFPFHRFYLYFHERILGSLIKDPDFAL
PFWNYDAPQGMEIPKIYTDKSSSLYDAFRDGRHQPPTLVDLDYNDVEPTISREKIIQCNL
SVMYRQVVSGARTPLLFFGQPYRSGSNPSPGMGTVENLPHNSIHLWTGDPNQSNRIDMG
TFFSAARDPIFYAHHANVDRFWSIWKSLGEKRQDIKDKDFLNASFVFYDENGEAVRVY
VKDCLDTRALGYVYDDTVPIPWLKTPPTPRVPRTPNKTKKKSTKKTGKLPSSVDKIVSFE
VKRPKKSRGTKEKDDEEEILVIDGIEFDGNKAIKFDVFINDEDDREIRADNSEFAGSFVNV
PHMKGSSSMNIKTCLRLGITELLESLDADNDDSIIVTLVPRFGDGSATVKDIRIEYDA
> PPO_MUT_protein
SEQ ID NO: 6
MASLSPSMPLALSSAAITTATTGGASFGLFYRKKKDPSSTIHRLNNLVVCSGS
NGSGEESNNSLWPGKFVDRREALIGLGGLYGSASSAFGVDPFALAAPVTTPDPSKCGSST
DLADGVKDLVCCPPSTNNVKPFLKPRVRKAAQSLDKEYIEKYKEAVALMKALPDDDPR
SFKQQALVHCAYCTGGYDQLGLPVELQVHFSWLFFPFHRFYLYFHERILGSLIKDPDFAL
PFWNYDCSARDGNTKNLHG
>HLS1_WT_gDNA
SEQ ID NO: 7
ATGGGGTTTAAAGGCTTTGTTATTCGAAGCTACGAAGAGAGTCAATTATC
AGATAAAGCTCAAGTTATGGATCTTGAACGAAGATGTGAAATTGGCCAATCAAAAC
GTGTGTTTCTCTTCACTGACACTTTGGGTGACCCCATTTGTAGGATACGTAACAGTCC
CATGTATAAAATGCTGGTAATCTAATTTAATTTTAATTAATTGTGTTTTTTTATAGGT
TAATTAATTATTAATTTGTGAATTGAAAATTTTTTATTATTAAGGTTGCTGAGCGGGA
CAAGGAAGTGGTTGGTGTTATTCAAGGCTCTATAAAACCGGTTTTTTTTACTGCTCAT
AAACCGCCGCCCGGTTTGGTGGTTAAACTGGGCTACATTCTTGGCCTGAGAGTGGCA
CCGCCGTATCGCCGCCGTGGAATTGGCTCTAGCCTCGTCCGCCGTTTGGAAGATTGG
TTCCTTTCTAATGATGTTGATTACTGTTGTATGGCCACTGAGAAAGATAATCATGCCT
CTCTTAATCTCTTCATCAATAATTTGAGGTATTTTCCATTTTTTTCTTTTTCTTTTAAGT
CAACAATTATGAATTGGGAGAGAGCACGGATCGAACAATCATTTTTAAAATGGTAA
TTAGTGTCATTTTATCTTATGTGTTATACTCAGATTAGCTATTAACTCTATCTTATAAT
GTAGGTTTTGTCAGTATTTTGAGAACTTTCAAATTTGTGTCTAACTAGTTTTTTTTTCC
TTAGTTACTTACCAAACACGTGATAATATTATTATATGATGAAATTTTTTATTTTTTTT
ATTTTTTTATTTATTTTTTATTGTGGCAAGTAGGAGCATCATACTTACCAACAACTTA
ATAGATATAAAATTAAAAATTTAATGGTCAAATCAAACTTTTTCGAGAGTAATTTTA
ATGTTGAGTTTTGCACTTTTAAATATAATTTACGTATGATTTTGAAGGATTAGATTCA
TGTTTAGTTTGAAGTGGTTTAGAATCTGGCAAAAGGGATTTTAACAATTTCAAACTA
ACGCCTCTCATATTTGTAGTTTGGGACGTCACTTTGCTATTAATTAACAAAATCACAT
TTTTGGAAATTAACATATGATTCACATGCAACTCGTAAATGAAGTTAAACTTGAAAG
TTTAGAGGCATATTAGAAATTTCTTTAAATATTCTTTTTCCCAACAGGTACATAAAGT
TTAGAACAGGAAGGATCTTGGTAAACCCAGTAAGAAATCATCCATACAATATGAAT
TCATCAGAAATCAACATTCAAAAGCTAAAAATAGAAGAAGCAGAAGCAATATACAA
AAAACACATGGCTTCAACAGAGTTCTTCCCCAAAGACATAAAAAACATATTGAAAA
ACAAGTTGAGTTTAGGGACATGGGTGGCAAATTTCAAACAACCGCCATGGTCGTCGT
CGAACTCTGTTGGAGGAAACGGGCAGACTATGGCGAGTAGCTGGGCCATTGTAAGT
CTATGGAACAGTGGGGAAGTTTTCAAGCTAAGGCTAGGAAAAGCACCATTTCCATG
GCTTATCTACACAAAGAGTTTAAAAATTATGGATAAAATTTTTCCTTGCTTTAAAGT
GGTTTTGGTGCCTAATTTTTTCAAGCCATTTGGGTTCTATTTTGTTTATGGATTGCACC
ATGAAGGCCCTTTTTCTGAGAGATTGGTTGGAGCTTTGTGCAAATTTGTGCACAATA
TGGCATTGAATAATTCAAAGGATAATTGTAAAGCTATTGTTACTGAGATTGGAGGTG
ATGAGGATGATGGGCTGAAAATGGAGATTCCTCATTGGAAATTGCTATCATGTTATG
AAGATTTTTGGTGCATAAAGTCCTTGAAAAGTAAGAGATATAATAATATTAGTAATG
ATAATGATAACGATAACGATCACGATCATCATATATTGGAATGGACAAATGCCTCAC
CTAATAGAACTCTCTTTGTAGACCCAAGAGAGGTATAA
>HLS1_WT_CDS
SEQ ID NO: 8
ATGGGGTTTAAAGGCTTTGTTATTCGAAGCTACGAAGAGAGTCAATTATC
AGATAAAGCTCAAGTTATGGATCTTGAACGAAGATGTGAAATTGGCCAATCAAAAC
GTGTGTTTCTCTTCACTGACACTTTGGGTGACCCCATTTGTAGGATACGTAACAGTCC
CATGTATAAAATGCTGGTTGCTGAGCGGGACAAGGAAGTGGTTGGTGTTATTCAAG
GCTCTATAAAACCGGTTTTTTTTACTGCTCATAAACCGCCGCCCGGTTTGGTGGTTAA
ACTGGGCTACATTCTTGGCCTGAGAGTGGCACCGCCGTATCGCCGCCGTGGAATTGG
CTCTAGCCTCGTCCGCCGTTTGGAAGATTGGTTCCTTTCTAATGATGTTGATTACTGT
TGTATGGCCACTGAGAAAGATAATCATGCCTCTCTTAATCTCTTCATCAATAATTTGA
GGTACATAAAGTTTAGAACAGGAAGGATCTTGGTAAACCCAGTAAGAAATCATCCA
TACAATATGAATTCATCAGAAATCAACATTCAAAAGCTAAAAATAGAAGAAGCAGA
AGCAATATACAAAAAACACATGGCTTCAACAGAGTTCTTCCCCAAAGACATAAAAA
ACATATTGAAAAACAAGTTGAGTTTAGGGACATGGGTGGCAAATTTCAAACAACCG
CCATGGTCGTCGTCGAACTCTGTTGGAGGAAACGGGCAGACTATGGCGAGTAGCTG
GGCCATTGTAAGTCTATGGAACAGTGGGGAAGTTTTCAAGCTAAGGCTAGGAAAAG
CACCATTTCCATGGCTTATCTACACAAAGAGTTTAAAAATTATGGATAAAATTTTTC
CTTGCTTTAAAGTGGTTTTGGTGCCTAATTTTTTCAAGCCATTTGGGTTCTATTTTGTT
TATGGATTGCACCATGAAGGCCCTTTTTCTGAGAGATTGGTTGGAGCTTTGTGCAAA
TTTGTGCACAATATGGCATTGAATAATTCAAAGGATAATTGTAAAGCTATTGTTACT
GAGATTGGAGGTGATGAGGATGATGGGCTGAAAATGGAGATTCCTCATTGGAAATT
GCTATCATGTTATGAAGATTTTTGGTGCATAAAGTCCTTGAAAAGTAAGAGATATAA
TAATATTAGTAATGATAATGATAACGATAACGATCACGATCATCATATATTGGAATG
GACAAATGCCTCACCTAATAGAACTCTCTTTGTAGACCCAAGAGAGGTATAA
>HLS1_WT_protein
SEQ ID NO: 9
MGFKGFVIRSYEESQLSDKAQVMDLERRCEIGQSKRVFLFTDTLGDPICRIRN
SPMYKMLVAERDKEVVGVIQGSIKPVFFTAHKPPPGLVVKLGYILGLRVAPPYRRRGIGS
SLVRRLEDWFLSNDVDYCCMATEKDNHASLNLFINNLRYIKFRTGRILVNPVRNHPYN
MNSSEINIQKLKIEEAEAIYKKHMASTEFFPKDIKNILKNKLSLGTWVANFKQPPWSSSNS
VGGNGQTMASSWAIVSLWNSGEVFKLRLGKAPFPWLIYTKSLKIMDKIFPCFKVVLVPN
FFKPFGFYFVYGLHHEGPFSERLVGALCKFVHNMALNNSKDNCKAIVTEIGGDEDDGLK
MEIPHWKLLSCYEDFWCIKSLKSKRYNNISNDNDNDNDHDHHILEWTNASPNRTLFVDP
REV
>BAG4_WT_gDNA
SEQ ID NO: 10
ATGAAAAAATGGTGTTCAAAAGGAAGCCAAATTAGGAGCGAAGAGTATG
GAAGAGGAGACGTAGATTGGGAGCTCCGACCAGGTGGAATGATTGTTCAGAAACGA
CATGTCGGGTCGGGTTCGGGTTCAAATTCGGAGCGTTTCATTACAATCAACGTATCT
CATGGGTCTTATCGTCATCAAATCACCGTCGATTCTCATTCCACATTTGGTATGTTAT
CATTTCAATTTGGGGGTTTTTTTGAAATACAGATTGATTTTTATTTTAAATTGAAGAC
TGAATTATTAATTTTTGTGTTTGGGACAGGGAATTTAAAGACAGTTTTACGACAGCA
GACAGGGTTAGAGCCGAGGGAACAGAGATTGTTGTTTAAAGGGAAGGAGAAGGAG
AACGACGAGTGGTTGCATATGGCCGGTGTGAACGACATGTCGAAACTCATACTCAT
GGAAGATCCTGCTACTAAAGAGAGGAAGCTTCAAGAGATGAAGAAGAAGAATACC
ACTGCTGCAGGCGAAGCACTGGCGGGGATCAGAGCGGAGGTCGATAAACTCTCCGA
AAAGGTTCGTTAAATCGTTAAATTACAACTTTAGTCGAAAAATATATTTGAGAAAAT
TGTAAAAACCACTCGTGGTAATTACAGTTATACCTTCAAACTTTTAATATTAAAAAT
TAAGTCTTTAAATTTATATTATTGTTAAAATTGGACTCTTAAATTTTGTTTAATTGTA
GAATTGAAGCTCAAAATGATAAAAATTGAACTCTCAAACTTATACAATTTTTACCAT
TTCTATTATTACTTAAGTTTGAGGTCTCAATTTTACCATAAAAAAATTTAAGAGGTGG
AATTGCAAGTTATAACTATTATCATACTTTAAGGTCAATTTTTACCATTTGTCTAGGA
TATTTTTTGGTTGGTATGGTTTATGTTTTTAAATTCTTAATTTCTCTAACATTTTGTGT
TTAATAAATGGGTAATAATTTATTTCATAAATTTTTTAAGTTCACACCTAAAATTCAA
TTTTATAACTAAAAAATTAATTAAATTTTACTTATTTATTTATTATGATATTCACATA
CTTTTAAGATATTTGAATTCTCAAGTGAATTTTTTTTTAAACAACAAGTTTTTCTGGA
AATTGACAAAAAAAAGAAAAAAGTAGTTTTAAATGCCTTGCTTTTATTTTATTTTATT
TAATGAAGTTTTGATAATGATACAAATGTTTATGTAACAAAAATGAAAACATTTGAA
AGAAAAAAATGGTTATTAGGTAACATTTCTAAAGTTTAAAAACCTATTTGAGACAAA
TGTGAAAGTTCAATAACTCATTGAATTGCTTTAGAAGGTTTAAAAAACAAATAGTTA
CAAACAAGCTTAGAGACTAAACTTCTAATTGAACCTAATTCTAAATGATTGAAATGA
ATTGACCAATGGATAACTAGCATATTATACATTTTTTTAAAAAAAAAAATTGGAAAG
GGGCAAAAAAGTGTGAGTGTATAAAATTAGGGTTTTTAAGTGCAGGTTGCGGCAGT
GGAAGGTAGTGTTAATGACGGGAAGAGGGTGGAAGAGAAAGAAGTTAATTTATTGA
TAGAGTTGTTGATGATGCAATTGTTGAAATTAGATGCAATTGAGACGGATGGGGATT
CCAAACTTCAAAGACGAACTCAGGTATCTATTGGACTATATGTCAATTATCATTAAA
AATAAATTTACTTTGGCTTATCTATTTATAATAATTAGGGTATACAAATTTAAGGTGA
TTTAAATCGTTTTCTTTCATTAATCTAACTATGCAAAACCGTTACAACAATACGTCAC
CTTTAAATAACTTTAACTATTTACCAAAACTTTATGAAGAGGAATTATAAATTTACTT
ACCGCCTAATTTCTCTTTTAAAACTCTTTTTGTTAACTCTTAATGTCGGGTATGTTTGC
ATTAGTCATATTTAATATCCATTAAATGATATAACTTTTCAAACAATAATAATTAAC
ATATATCTTTATTATTATTATTAGTTATTAGATTTGTATAGTTTTCTAAAAAAAAGAA
TGGATTTTATGTAAGTTTGGATTAACTTAAAAAATAAATATTTTTTTAAAAATTATTT
TTATTTAAATTTTTGTGACAAAAACTTTTTAAAATAAAAACACTAAATTCCATTTGGA
TGAATTATATATTTAAATATTATATTTCTATGGTAGAAAATTATTTACTATTATTTAA
TCAATTTTAATAATGATGGATTAAATTTAAGTTTTATTAAATGAATACTTGAAAATAT
GAATTAAAATTAAATATATATATTTTAATTTTTCAATTTTGGTATTTATAATAAAAAG
TACGATAGTTTAATCATTAATGGGTTAGGTGGCTGGTGCTCCCCTAGGGCCATCAAA
CTTAAAATAATTAAAAATAATGAAAGTCTCCTAAATTGTATGAAAATTCAATGAATA
TAAATTGTGAAAAATGATAATGGGTATTTTATCTATTTATTTATTAACTCAAAAAAA
AAAATTAATAAATATAGACTAAAAAAATTGCAGAAATAGGACAAAATGATTTTAAT
TCTTTCCCTTGATATGACATTTTTATGTGGGACATTATGAAACCAAGAACTTATCAAG
AAGGATTCTATTCAAAATAAATAAATAATTGATTAAAGAAGAAAATTCCATTAATGT
CCCTAAAGTCTTAATCACACCTCTATTTAGCGTCTATCATGAATAAAATAAATAGAA
ATCATAGGAATGCTGAGGTGGCATGAACACTAGATAAAAATTTTAGGTTTAAATACT
ACTTTAGTTTTTATATTTTTCACATTGTTTCATTTAGATTCACGACCTTTTAATTTTGG
TTAAATTATAAATTTAGTCCATATAATTTGAAGAAAGTTAAAATTTAATCCTATAGTT
TATAATTAGAATTTAATCTCTATGATCTGATAAAATCCTCATAAATAATCTCACTACT
GTAGAGACTAAACTATAGGGAACATTTATAAGGTTTTATCAAACCATAGGAACTAAT
TCTAGATTTTAAAACCAAATGGACCAGATTTTAATTTTCTCCAAACTACAGGGGCCA
AATTCTAATTTTTTCTAAATTATAGGAGACAAATTTGCAATTTAACCTTTAACTATAG
TTAATTTTGGTCCACTTACTTTCAAAATATCAATTTTAGTCCCGTGGTTTTAAAAAGT
CTCCATTTTGGCCCCTTAACAATGAACAAAAATAAGATAAAAATAGTAATTAAATTT
TAATTTTGAACTATGTAATTTTTTTTTTGAAGTACAAATAGTAGAGTAGGGAAATTG
AGAGAAAGAGTATACGTTAATTATCATTGAACTATGTTTATTTTGGTGGTGATAAGT
TTTTACGCAATTTCAATTAATTTGAATAACGTTAGAATTGTAATTTTATAATTTTGGG
AATAAAACAGGTTGTTAGGGTACAGAAATTAGTGGACAGAATTGACAAGTTGAAGG
TTAGAATCTCAAATCCTTTAAACCAAACAACAATGAAAAGAGGCAAATGGGAGGAA
TTTGAATCTGGATTTGGCAGCCTTATTCCTCCAACTTCAAAACTCACCATCAGCTCTA
CAAAAATAACTCATGATTGGGAACTCTTTGATTAG
>BAG4_WT_CDS
SEQ ID NO: 11
ATGAAAAAATGGTGTTCAAAAGGAAGCCAAATTAGGAGCGAAGAGTATG
GAAGAGGAGACGTAGATTGGGAGCTCCGACCAGGTGGAATGATTGTTCAGAAACGA
CATGTCGGGTCGGGTTCGGGTTCAAATTCGGAGCGTTTCATTACAATCAACGTATCT
CATGGGTCTTATCGTCATCAAATCACCGTCGATTCTCATTCCACATTTGGGAATTTAA
AGACAGTTTTACGACAGCAGACAGGGTTAGAGCCGAGGGAACAGAGATTGTTGTTT
AAAGGGAAGGAGAAGGAGAACGACGAGTGGTTGCATATGGCCGGTGTGAACGACA
TGTCGAAACTCATACTCATGGAAGATCCTGCTACTAAAGAGAGGAAGCTTCAAGAG
ATGAAGAAGAAGAATACCACTGCTGCAGGCGAAGCACTGGCGGGGATCAGAGCGG
AGGTCGATAAACTCTCCGAAAAGGTTGCGGCAGTGGAAGGTAGTGTTAATGACGGG
AAGAGGGTGGAAGAGAAAGAAGTTAATTTATTGATAGAGTTGTTGATGATGCAATT
GTTGAAATTAGATGCAATTGAGACGGATGGGGATTCCAAACTTCAAAGACGAACTC
AGGTTGTTAGGGTACAGAAATTAGTGGACAGAATTGACAAGTTGAAGGTTAGAATC
TCAAATCCTTTAAACCAAACAACAATGAAAAGAGGCAAATGGGAGGAATTTGAATC
TGGATTTGGCAGCCTTATTCCTCCAACTTCAAAACTCACCATCAGCTCTACAAAAAT
AACTCATGATTGGGAACTCTTTGATTAG
>BAG4_protein
SEQ ID NO: 12
MKKWCSKGSQIRSEEYGRGDVDWELRPGGMIVQKRHVGSGSGSNSERFITIN
VSHGSYRHQITVDSHSTFGNLKTVLRQQTGLEPREQRLLFKGKEKENDEWLHMAGVND
MSKLILMEDPATKERKLQEMKKKNTTAAGEALAGIRAEVDKLSEKVAAVEGSVNDGK
RVEEKEVNLLIELLMMQLLKLDAIETDGDSKLQRRTQVVRVQKLVDRIDKLKVRISNPL
NQTTMKRGKWEEFESGFGSLIPPTSKLTISSTKITHDWELFD
>cl_97103_v1_Chr2:29897185-29920517
SEQ ID NO: 13
TTGTTACCTAATAAGAAATTTAGACTTTAGTATTTAGTTTTTGAAAATTAA
GTTTATAATATTACTTTCACTTCTAAATTATATGTTCTGTTGTCACTCTTTTATCAATA
TTTTTAAAGTTTCATTTAATGACCATTTGATTTTTAGTTTTTGAAAATTAAGTTTATAA
ACATAACTTTTACCTATTGGTTTCTTTGTTTTGAAGTAAGTTTTGAAAACTAAAAACT
TAAAAAAAGTCATTTCTAATAATTTTTTTTTCTGGAAATTGGTTAAGAATTTAAGTGT
TCAGTAAAGGAAGACGAAAACCAAGTAACCATGATAAGAAAGAATGTAAGAAAAT
AAACATAATTTTTAAAAACTCAAAACTAAATAGTTATCAAACCAAAAACTAGTAATT
CAAATAAATATATATATAAGTACTTGAACTAGCCCCAACCAAAGTACTTATTTGATT
GAGTAAACTACAATTAAATTAAAAGGTTAAAATATTTAATTAATCCTTAATTAACTA
AAAATATCAATATTGACAACTCATTATGGACCATATTACTCTCTCTCTCTCTCTTAAT
AAAAGAAACTTAATATAAGTTGAAGGTTGGTCCATTGGTTTATTTTATTTTAAATCTT
TTTAGCCCTTCTTAAATTTTTAATTTTATAAAAACAAATTTATTAGTCACCAAAAAAT
GGTTGCTTGTAGGATGAATGAAGACGAAATACTAAATAATTTTATTTTATTACCACG
CGTGGCCACAGAAATGTTATTTTTAAAATTTTAATATAAAGTTTGTGAGGCCAATTA
TTTTTTAATTATGCTAAAAACATTCAGCTTTGCGTGAGTTGGAACTTGGAATACACA
ACATATTATTTGACCATTGAAAATCCAAAATCTGAATCACAACCCTCTAATTTTATTC
TGTTATTATTATTTATTTGGATTGAAGTAATCCAGCCATTCCTTTACTTATTTATTTAT
TTAATATAAGGTAAGTGTTACAAAATCGATAAACAAAGAATACAAAAAATATAAAA
AGATAGAGAATCGACATATAGATTTACATGATTTACTAACAGTGTGTTAGTTACGTT
CACAGAACAGATGAAACACAATTTTATTAGAGATAATGTTGCAGAATACAATACAG
TGACACCTCTATCTTTAGAGAATTTATATAGTGCACTCATTTAAACCTTAGGGATCAT
AATCGTAATTAACCATAAATAATTAAATATATCAAATACGAATGCCCCCAAATCCCC
ACCAGTAAGATCCCATACTTAGTCATTTGAAGTGCCAGATAGCATACAACATTATTC
AAACAATATGTTAGTTGACCACCTTGACTTTATTGAAAAAGGATTCAAATCGAACCT
AGTAAGATTGCATTTACAATAGATTAAGAAAGTTCATGAAAGAATAACACAATTATT
TTCTTTCCAAAAAACACACACAATAATTTTACGTGGAAACCCTCTAAACAATTTAAG
GCAAAAACTATGGATAAAGATAAAAGAATTTCACTATATAAAATAGGTATTACAAT
TTGTTTACAGACTCTCTCGTAAGATAAAATCTCTCTATTTTATATCTCAATCTTTTCTC
ACTTAATTCTGGTTTGTTAAGCAACCATGGGTTCTGACCTCGTTATAACTAAAAGAA
ATTTAGCCATCAAACAAATTCAATGGTCCCAAAATGACTTTGAATAATGTGAAAGTT
TTAAGGGTCACAAATTGACCTTGAATGGAGTGTAAAATCTAATTAACCACAAATTGG
CCTACAAGAAAATGTATTGAATAATCTTGAAGATAATATTATTTAACTAACTTCTCA
TAAGAAAAATTGTTATTTTAATTGTTGAGTCGCTCACGTTGAAAGTTTAAATCATTTC
AATTATTATGAGAATGCTATTTTTTCTCCTTTTATTTTAATGAATAATTTGAGCACAA
CTCAGTTAGTTAAGGTATATATTCTCGACCAAGAAATCAGACATTCAAATCCTCTCC
CCTTACATATAGGATAAGAAAAACAATAAAGTTTAGAGGCATATTAGAAGTTCAAA
TACTTATTAGAAACAAATTAAAATTTTATTGTTCAATTAGACACACCTTAAATTTATA
ACTAATAGATCAATCATTTAAAAGTTTCGAACGTGTCAAAAATTTATTGTGGCACAA
ATTTTTTAAAACAATTAAAATACTAAAGATTAAATACTATTTTGACTCACTACTTTGA
AGTTAAATTTATTTTGGTTTTCATACTTAGAAAAATGTTGACTTTGATTTCTTTAGTTT
TCATGTTTGGTTTATTCTACTCTTACACTTTTTAAACCTTCGTTTTAATTTCAATTTTTT
TTTCTTTTAAAAATGTGAAATGTTTTGTCCTTTTTTTATTATTATATTATTTTAAGAAT
AATAATAAACTCATGATTTAGAATTTAATTTCAGCGTAACACTACGAGCGATTTGAT
TTGAAATGTAATTATAACCGAGGGGTAAATTTCGCGGCCCATTTAACAGACCATTTA
CAAAACTTGAGCCGGGCTGCCACCATGTGGGCTGGGTGTCAAATGCAACTGGTGAA
GTGGCCTGCTGATGGGCCGCTCCATCCAACCCATTCAAACCTTAAAAAAGAGTTAAA
AAATATTACTACGGTTAGTTTTGGACGAAATCCTATGAACTTTCAAAATTATAAAAA
ATATTCTTCAACTTAAAAAAAAAAAAAAAAAAACTATCTTTCCTATTAATATATAAA
TAGAAACTATTTATACGTCGTTGCAAAAATAAATATGATATTTGAGATTTTTTTTTTT
AAATGCTCGTATAGTATATAATTTTTTTTTTCTTCATATAGACTATTCTTACATCTATT
TTCTCAGAAACCAAATTAAAAACATAACTTTAAGTTAAAACATAAAATCTCACAAA
ATTCAAAAATCAAAATCTCTCAAAACACACCAAACACTTAACTACTTATGTACAGTT
TTATTAAATCATTCAAGTCGTAAAACCCTTTGATGCTTCCATTATTCTTTTTCTTCTTA
TTAGAATCTATGTAACTTTAATTGAAGTGAGCATATTTAGAGGGTATTGAAGAAAAG
AAAGAAGAAAAAACAGGGAATAGTAAAAGTATTTTTAAACTATTTTTCAAGGTTTA
AGAAAATTTTTGAAAGTTTTGGAAGTTTAAAGGTATTTTAGCACAAATTACATGTTT
TATTTTTAAATTGTAGATTTCATAAACTTCCGTCGTTTTCTCTCCTTCATTTTCGTAAA
CAAAAATCGTTTTCAAAATTCAATTGTTATCCACATAAAATTTATCAAATAATTTTTT
GTTTGTTTGTTCAAACCATTATTTAAGCTGCAAATTTTAATTTTTACAGTTTTGGTTG
ACACAAAACAAAACTTGAAATTACCTCAACATGCATATAAACACTAGTTTTGAGCCA
CAAATCACTATGGATCTATAGTGGAGTGGAATAAAGGTATATGACATAAATTTTTAG
CATAAAAATTATCTACTATTATTTTTGGTTGGTCTCCTTTTCTTATTTGATCATGCATT
TTCAACATAAGCATTTTTCATACTTGTTTTGCTCCGATGCATTTTTATATTATTCGATG
GGAGGTGGGAGAAAAATAATAAGTATCTTGACTTCATATGACTCAAGTAAAACTTC
GGATTCAACCCTACAACATTTCACTAGTTGTATTTAGGGGCTAGCTTACATTTTGAA
ATAAGTAGGTAATAATTAATTGCTTGACTATGTATAGTGATTGTAATAAGGCTTAGC
AATAATAAAAGATGGTTTATGATTTCTATTTTTCCTTTCTAAAGTATGGCCGATACCA
TTTTCATAAACGTTAACCATTACAAGAGATTGATATAATAACATTGCTAACGTTGTT
AACAAATGTATTATCTATACAAAATAATGTTTTCAACAATTACAATAGGGAAGAAA
ACATGGAAAAGTAGTGCTTCACACTTTGGGCAAATCTTGTTTAATTAATTAATCTTGT
TTCGAATATAATAATGCAATATTTTCTTTTTATCAGACTTAAAATTTATCGATTATTT
GGGCTGATATTTTGCATAATTATTTTTATAATTTTAAAATTAGATACTTGATCAAGTT
GTTAGAATCTTTCTTGTTTACTAGATTTTCTCCAACTTCTATGATTTGTTTGGTTTTCA
ATATTCATGGCTTTTAGATTTGATACGAGGGTATTTCTGTCCATCAATTTTCTAGTTG
CTATATAATTTACTTTTCAAATATTTGAACTAAATACTGATCTATTTGATATATTAAT
TATTTTGTTTCTATCATGATGTTTTTTTTTTTTTTTTTTTTTATAATTTTTAATGGCTTG
GTATTCTCCAAGTGCAATTAACCATTTAAATGAAAATTTTGGTAAAAATTTACTATTT
GATTAAAATAATTTGAAAATCAAGTTTATTTTGAGATATAACCTCATGCTTATCTAA
CATGGTATCATAGTTATAAAGTTGATATTCAATCTCAATAAAAAAAAAAAAAAAAA
AAAAAGAAACATTGAGATCCAATTCAATAAGAGTGAACCAAAAGATCCACCATCTC
AAAGGGCATGTTGAGTGGTCATACTTTGAAAAAATTGAGAGACTTCACGTGAAGAT
ACTTAAATTAATTAATTAATATTTTAATGATAACAAATCTTATTTTATATTTAATGTT
TTGAAGAGTTTATTGTTTTGAGTTCAGAATAATGATTCCAACGTAATAATTACTCAA
ATAGTAGTTCAAAAGAAGAAAATATAACAAACATTCTAAGTTAACCAATTGAAATA
TGACATTTGAAGTATATGGGAAAAATGACACATGATGGTTTTTTGACTTTTAAATTG
ATTTAAAAAAAATAAATAAATTTAATATTATATAGGTTTATGTTCTATCCACTAGTTG
ACTTAAAAATTAATAATAATAATTAAAAAAAAAAAAAAGTGCATGTAGCTGTTGGA
GATCACTTTAAAGTAGAAGAATTTTTTTTTTTTAATTTTTAATCTAAACCATTTAATA
TTCTCTTTAGGATGATTTAAATACTCAATGAATTTGGATTTCACTATTCTCTTTTGAA
ACTCTTCATCTTTTTCACATTTCAACAACAATTTTCATAATTTTTAATTCTCTAAAAA
AAATACAAAGCTTCCATTGTTACTTTCTTCAAGCTTCATTTTTTTTTTCATCTTATTTT
TAAGAGAGATTTATGTATTGTGAGAATTGATTTGTTTTATAAGCTCAAATTTGTGAAT
CCTACTTCTTTAAAGAGTTTTTCTCATCTTGTTCTTCATTTTCAAATTCATATTGTAAG
TGGTTACTCTTGAACCTGTGAGAAGAGTGTGGTAACAATTTGATCATCAATGGTGGA
AAACATCGAGTTTATTCTTGTGCCTGGAAAAAGAACGTTGTAGCAGTTCACCTCAAG
CTGTGGAATGAATCGAGTTTGAGTGACCATATCCAAGAGAAACTTAGGGAGTGGAT
GTAGGTCGGGTAGTGTCAAACTACTATAAAATGTGTCAATTTCCTCTCTACGTCTTCT
AATTTATTTTTGCAATTAATTTGTTATTATATTTTATTCCATGAAATTAATTGCAATAT
TTATTCCTTAAATTAAGTGCTTACACATTGATTTATTTAGATGGGTTGAATTGATCTT
TACATATCCTTTACCAATCTTCTTGAATCAAATAGGGTTCTATAAAATATTAGTGTTA
ATTTATTATCTAATTCAAGATGAATTTATTTGCATGAAATTATTGTTTAAGTTACTTG
CTCGCAAAAAGTATATTGATTTGTTTAATTGGGCCCACTGTAGTAAAAAGTTTTAAT
TAATTTAATAAACTCTATTCACCCCTTTAAGGTTGCCATACCAGTCCTACAATGAGAT
GGAACTCATGTACGTTTATCTAATACCACCCTAATGCCTGCAAAATTTATAACGAGG
GGTGAGAAATGAGAAAGAGGCGATCTCCCATTTCCGTCCCCGCCCCATCTCGTAAAC
ATCCCTAATATATAATCCAAATGCATTTTTCTCACAAGAATGAATTCTCTTTCAAGTG
GTCCAAGAGGGAAGAAGTTTGTTTTCAAATAACGTAATCAACGCAAGCTGAAATAA
AACAGCAGCCATTTGAGAATCAAACACAAAAAATGGAAAATTTGTATGTGTATAAT
TAATTAACCATCAAAATTTCATAAACAAATAAACACTTCAAATCAACTTCACATTGT
CCATAAATTGCAAAGACATATTTCCATTTTAGGAAGAATAAAGTTTTGGCCTAACTC
ATCCATGGCTGTGTTTTTTAAAAGCCCTAATAATAATATATTCCAAAACTCTCCTCTT
TATCTTTTTTCTCAAATCTCTCCCCTTTTTGCCCTTTGCCCTTCCCCCACCTGGCCTTC
CTTTAAACCCTAATAATTCCAAACTCTCACCTTTTTTAAGCTCAACACTCAATATTTA
ACCTAATCTCTATTTATATGTATATCTTTCTTCTTTTTCACTCTTATTATTGCCTTTGC
ATATAGCACATGTGATGAGAATATTCCCCAAATGCCTCTCCTCTTATTGTTTTTCAAC
TTTGAATCTTTATAAAGTAAAAAGATGGGTGACACCTAAATTAGGAATGTCTAAAGG
TTTTTTTCTCTTAAATGACACACAAAAAAACATAATAATAAGATGCAAAATCTAGAC
ATTTCAATCCACAAAAGAATTAACATTATCGTTTGATTATTAATTATTTGGACTCCAA
AGCAATAGTACATATACTATTAATAGAAAACGACGTTGATATTTTTTGCATTCATAT
TTAGTCATGAATACAATAATACGAATAGAGACCTAGTTAAATCCAGAAACATCCAC
GTACAAAAAAAAACAAAATTTTACAACATAACTATTTCAGTTCAGATCGAAATCGTC
TTCTTCTTTCTTTATAACTTTTCTTCTACTTTCTTCACTCTGCTATCAAGTACAAACAA
TAATTAGATTGAGACATATCGAGAGAGGGTTAAACCTATTTTCTTTTTATTCTTTTAT
ACCTCTCTTGGGTCTACAAAGAGAGTTCTATTAGGTGAGGCATTTGTCCATTCCAAT
ATATGATGATCGTGATCGTTATCGTTATCATTATCATTACTAATATTATTATATCTCT
TACTTTTCAAGGACTTTATGCACCAAAAATCTTCATAACATGATAGCAATTTCCAAT
GAGGAATCTCCATTTTCAGCCCATCATCCTCATCACCTCCAATCTCAGTAACAATAG
CTTTACAATTATCCTTTGAATTATTCAATGCCATATTGTGCACAAATTTGCACAAAGC
TCCAACCAATCTCTCAGAAAAAGGGCCTTCATGGTGCAATCCATAAACAAAATAGA
ACCCAAATGGCTTGAAAAAATTAGGCACCAAAACCACTTTAAAGCAAGGAAAAATT
TTATCCATAATTTTTAAACTCTTTGTGTAGATAAGCCATGGAAATGGTGCTTTTCCTA
GCCTTAGCTTGAAAACTTCCCCACTGTTCCATAGACTTACAATGGCCCAGCTACTCG
CCATAGTCTGCCCGTTTCCTCCAACAGAGTTCGACGACGACCATGGCGGTTGTTTGA
AATTTGCCACCCATGTCCCTAAACTCAACTTGTTTTTCAATATGTTTTTTATGTCTTTG
GGGAAGAACTCTGTTGAAGCCATGTGTTTTTTGTATATTGCTTCTGCTTCTTCTATTTT
TAGCTTTTGAATGTTGATTTCTGATGAATTCATATTGTATGGATGATTTCTTACTGGG
TTTACCAAGATCCTTCCTGTTCTAAACTTTATGTACCTGTTGGGAAAAAGAATATTTA
AAGAAATTTCTAATATGCCTCTAAACTTTCAAGTTTAACTTCATTTACGAGTTGCATG
TGAATCATATGTTAATTTCCAAAAATGTGATTTTGTTAATTAATAGCAAAGTGACGT
CCCAAACTACAAATATGAGAGGCGTTAGTTTGAAATTGTTAAAATCCCTTTTGCCAG
ATTCTAAACCACTTCAAACTAAACATGAATCTAATCCTTCAAAATCATACGTAAATT
ATATTTAAAAGTGCAAAACTCAACATTAAAATTACTCTCGAAAAAGTTTGATTTGAC
CATTAAATTTTTAATTTTATATCTATTAAGTTGTTGGTAAGTATGATGCTCCTACTTG
CCACAATAAAAAATAAATAAAAAAATAAAAAAAATAAAAAATTTCATCATATAATA
ATATTATCACGTGTTTGGTAAGTAACTAAGGAAAAAAAAACTAGTTAGACACAAAT
TTGAAAGTTCTCAAAATACTGACAAAACCTACATTATAAGATAGAGTTAATAGCTAA
TCTGAGTATAACACATAAGATAAAATGACACTAATTACCATTTTAAAAATGATTGTT
CGATCCGTGCTCTCTCCCAATTCATAATTGTTGACTTAAAAGAAAAAGAAAAAAATG
GAAAATACCTCAAATTATTGATGAAGAGATTAAGAGAGGCATGATTATCTTTCTCAG
TGGCCATACAACAGTAATCAACATCATTAGAAAGGAACCAATCTTCCAAACGGCGG
ACGAGGCTAGAGCCAATTCCACGGCGGCGATACGGCGGTGCCACTCTCAGGCCAAG
AATGTAGCCCAGTTTAACCACCAAACCGGGCGGCGGTTTATGAGCAGTAAAAAAAA
CCGGTTTTATAGAGCCTTGAATAACACCAACCACTTCCTTGTCCCGCTCAGCAACCT
TAATAATAAAAAATTTTCAATTCACAAATTAATAATTAATTAACCTATAAAAAAACA
CAATTAATTAAAATTAAATTAGATTACCAGCATTTTATACATGGGACTGTTACGTAT
CCTACAAATGGGGTCACCCAAAGTGTCAGTGAAGAGAAACACACGTTTTGATTGGC
CAATTTCACATCTTCGTTCAAGATCCATAACTTGAGCTTTATCTGATAATTGACTCTC
TTCGTAGCTTCGAATAACAAAGCCTTTAAACCCCATTAATTAGAAAAAACAAAATTA
AAAGATAAAGATTGAGAGTGAGTGAGTGATAAAAATGAGAGGAGAAATTTATTTAT
ATAATAAGGGGAGAGAGAAGAAGGGTTTGTTGTAAATATGGTATTTGATATCAACA
TCAAGATGGTAGGATAAATTTTGAAAAGGTGTTTAATACGAGACAAAAAGTGATTA
ATGATAGAACGTGTTCCTTGTCTATGGTTTACCCTTTCTTAACTTTTCTTTTACTTATT
ATAGGCAGACCATGTTAGGATAACTATATGCCCTCACAAAATTCTCTCTCTCTCTCTC
TCTCTTGTTTATTCAATTAACATCTCTTTTCTATTTTAATTTAATAAAATACAATTAAC
TTATGATTCACTCAATTTTCTCACTCAACACATTGAAAATGACCATCTTAATCCCTGT
TTGCAAAACGTTGATATAAAATTTTGAAAAAAAAAAAGACTAAAAATGAAGATTGA
TGGACCAAAATTGTCAACCAAAATATATTTTGAAAGTTCAAGAATCAAATTGAACAT
TTTGGATTAAAATAGATTAAATTGCAAATTTGGTCGTTATGGTTAGGAGGACTAAAT
TCTAATTTTTTTCAAAAAATCATAGGGATCAATTTTATAATTTGACCATTGAAATACC
ATTTTAATCTACGTATTTTGAGTATAATTCCATTTTAATACATATACTTTCATTTGCGT
AAATTTAATCTTAAATTTTCTTTTATGAAAATATATTAAAAATAATAATATATTATCA
AAATTTAGAACATAAGGCTAATACAAAGCAGAATAAGTAAATGGAAGCGTATATGA
AAATAATATTTAAATAAAATAAAATAAAATAATAATAGAGATGAAAATGAAAGCTA
AGAAAGAATGGAATTGGAAAAGATTGTATTACAAAGACATAATGAGTACTTTTTTCC
GTTACCACCTTTTGAAAAATGAGGAAAACTAGAAATTCTTACCTTAAAAAACGCCAT
ATATACAAAGGGCAAAAACAGATCGATGACAAGAAAATTCCAATTTCAAGGCCAAT
CAATTTTTCGCTTTCTTTTTATTAAAAACAAATAATTATTATTATTATTATTATTATTT
TTCCTTTGTCAAGCATTGATTGATTCACATTTTAATGGAAAGGATGGGATTTCGTTGA
AAATGGGTTTAAATTAATGGTTAATTTGGGGTGGTCCACTCCATTGCTTCCATAATTT
CTTAGCTTTTAAAAGACATTTATGAGATATAAATTTAGATATTGCCTAAGAAAGTGA
AAGTGAAATGGCCGTAAATGAAGCCCATATTTATTTTGTAGTTACTAGATTAAGTCC
TATTTGGTAATCATTTCGTTTTTGTTTTAAATTCATCTCTAAGTTTCAAGATTTCACTA
AATACTCACTTCCAAAATAATTTTGGAGTTAATATTTTTTTAGTTAATTTAAAATAAT
TATGAAGTAAAATTTTAAAATTAATTTAAATAGTAATGGACAAATAGTGAAACTTAT
TCAATTTTAATTCTTTTAAATAAATTAATAGATAATTAACAATAAAGGCTAAAAGTG
AGTATTTTGTGATAAATCTAAGTTAAAAATATAAGTCTTCAAACATAGGAACCAAAT
TGAAACAAAGTTTAAATCCCAATGGTAAACTTGTAATATTTTGAAATTTAGGGACTA
AACTGAAATCAAACTCTTTAAAATCTATGGACCAAATGAAAACTAAACGTAAAACT
TAAGGTCCAAAAAAGAGTATACGAATGTGTTAGTGACCAAAAACTAGTCTGTATTTT
GAATTTTTCTATCTTTATTGTATTGAAAAAAAAAAAATTCCTTCTATTGAGGAACTAT
TTATTTTTAATTTGTTAAGTGGAAAAATGATATGATCGTAAGGGGGCCCCTCTTTCAC
ACCATGTCTTTGGCAAACGTGTGGTAGTGAATGATAAGAAAGCCAATAGGTGCAAT
AATATGCCTATAGAAACTTTTATGTCAATTGCATTTTTTTCCCCCTTAGTACAATGGG
AATTTAATTTGATGACATGATTTTTCAAGTCTCAATCAAGTTAGGATTCTTGATTTGA
ATATTATCCTAATTAAGGTATTATTTAAGGAGCTCGGTGCAATATAGTTAACGATAT
CATATTGATGGAGACAAAATTATTGCTTTATCATACGTTTAATAATTTAGAAGATAA
CGTTTACAATATAACATCACAGTCCATATAAATAATTGTTATTACGTACTTTTTCTTT
TCAAATATTACAATAGTTATGGAGAGAATACAAACTGAACCATTTTTTTAGTCTAAC
AATTGCAAGGAATGGGAACTAAATTGTTGACCTTTGATGTAATCAATTAAAGAATTA
AAAGAGAGAAAACCAAAAAGAGGAAGGAGTGTACAATGTGTCGAGTAATAGTTTC
GATTGAATGAATGATTAGTGAGGGGTTGTAATAAGGTATTTTTAGGCATAAGGGGG
GTGAAAAATGGCAGCCTGGCACTTTTTTTCTAATATTTCCAATGATTTCTGTGGACCA
CTCCCCTTCCCCTCTCCCTACATGTTTTTAGAAAAAATTACCAATAAGGAATCCAAC
CCTAATTATTTCTTCCATTTCTTATTATAAACCTTTTCTCTCCCTTCATATTTCATTATC
ATTATTATTTGTTTTTCTAAGTGGGGATAATTTTTTATTTTTTATTTTTTTAATATATA
TATACTTTTTTTTTTTTTTATGTTCATTGGCCCTCTCTTTTTAAAATGTTTCTAAAATTA
TGATAGTTTTAGACCAAGTTAAATGTCCTCAAAACACAATTTCATGAATATAGCGAT
TTTGTTTGATGAATGAAATTTGAAAATATTTTATTTTTTTTCTAAAAATGAAATTGTT
CCATTAATAATTTGCTCTTCAATGTATAGATGAAAAAGTCTAACTTCATTGATTGTCA
AATCATCCTTTTTTATTTTCAATGTTATATTAATGTTTAAAATTTTTCGATAAATGTCA
AGTTGATCAAGTGATATATATCTAATCTCATTTGAGCCATTAGAAATCTTCTTATAAA
CCAATAGTGTCAATCAAGCTATTAAAAGAGATGTTATTGATGATGATGTTTGACAAT
TTAATTTTTTATGTCTAGAAATCTTCTTTAGAAGCTGGGATTTTCTCTATATTATTCCA
ATCTTCTTTATAATTTTTTAAAAATATTAATTACATTTCATTTTTCTAAAAATATAGTA
AATATAAAAAATAAAAAAAAATCCCTTTGAAGTTTTTCACTCTATTTACAATTACAT
TCTTTTAAAAGTCGTAATATTACTTTTGTACTTTTATAAGTTCATTTTCCTTCTCTCTC
TTATTATTTTGTTAGTATTTATCGTTATATTTTTTTAGCACAATAATTGTGGGATTCGA
ACTTTTGACTTTTTTTAAGAAAAAAAATCTACGTCTTATGTCACTTGAACTATGC TCA
TGTTGGCTTATAATGATGGTATAATTTTTTCATAGTTAGAATTCTATCGCTAACATAC
TTTTAATTCATAACCTAAATCTATCAATAATAGAGTATTATGATGGATAGATTTCAAT
CACATTGCACAACTAACATATATATATATATATATATATATATAATTTATCTCTTCTA
TCAAAGATAGATTTCAAACAAATAATTTTCCTTTTTGTTTCCCTCCATTTTCCTCCAA
TTTTATTCTCTCATCCCTTTCATTAATAACCCAACAAAATATTCAAAGTAATTTGTTA
CAATATCCTTTTCAAAGTAACTAGAAGAACTAGAAACAACTAAATTGCACTTAAACT
GTCTATGACTGAAGCTTAAAAGTACATAATAAAAATCTATAAGTAGTCTTATATGTA
AGTGATAGATTTTAATTTGCTATTGGTGATACCTCATATCATTGATAGGGTCTACTCA
TGATAGAGAGTAAATGATAAACTCTATCATTGATACCAATCTATCAATGATATACTT
CTATTGTTGATATATTTCAAGTAATATAAGCCTACAAGTCATAGACTTCTATTACCGA
TAGACTCTATCACTAATAAACTTCGATAGTTAAATCTAAGTTTTGGTATATCTATAAA
TTCTATTATATTGATATATTATATATATTAATACTTTGAACATTGTTGTATTTACAAC
AATAATATACTTCAAATTACTAAGATAGCAGCCGAACATCAGAACCCATGGGCTTG
GGCCCAATAATATCATGGCACGAAGTACAACCCCATGGACATCGCATGGGTTATTA
AGCCCACCAAGAGCCTAAATCACATCAAGTTCAAGCCATGATCAGAGGCCTCAAGA
AGCCCACGGGTATATGTGGGCCAGCAAAGCCCAAAAAACTTGGGCCAAAGGCCCAA
TTAAAGGAATTGTCGTACGATGTGGACAGTTGGACATGTACAATTCCCTTTTTAATC
ACGAACTTTAAAGTTGTGACCGACCAACCGCTGTTCGTAATGACTGTTTGAAGGTCC
CTTTCATCAATCCACTGACACTTGAAAAGTCATTAAAATCTCCGTCGCTATTATGTAG
CTGTCTCATACTAATTTAACCTAACTTTTAAACTTTAGGCTAAGTTTACATGTTATTA
ACTTTCAAATTTTACATCTATTAGGTGTCCTCAACTTTTAATTAAGTGTCTTAATGAG
ATTTTAGTTTTTAATACATTCTTAAATTTTCAATTTTACGTTGAATATATATTTAAGAC
CTTATAAACACTTATTCTAACTCAAAATTTATTGATTTGTTATCTACTTTTTTTAATAT
ATTTTTTTAAAATCAATCTAAATTTTGAAATCTAAAAAGATTTATTATTATTTATTTTT
TTATTTTTAAAAAAGCTTGTTTTTGGAATTTGGAAATTGAGGGATGCTTAATTTCAAA
AAAAAAAAAAAAAAATGAAATGGTTATCGAACAAAACTTGACCTTTTAAATTCATG
TATCTATTAAACATAATGTGAACTTTTACCGTTTCTTTAGATATAAGATCCAATTTTA
TGTCAAATAGGTTAATTACAAATGAATTAAGGGTAGTAGTTTTAAATAGCAAAACTA
CTAGAAATATTTACAAGTATAGAAAAATGTCTCTGTTTATTAGTAATAGACAATATT
GATAGACATGTACCAGTGTCTATTACTAATAGACAATTATAGATGTTAGTAATCTAT
CAGTGATAAATATGATATTTTGCTATATTTTAAAATTTTTTTATATTTGAAAATAACT
CATGAATTAATTTATCAAATACTTATTATATACAAAATTATAAGTTTAAAAATTTATT
AGACACAAAATTTAAAGTTTAACTTATTAGACACATCTATCTATTAGTGTCCGGTTG
GGTTTGGATTCAACAGTTTCGTTATATCTAAAAATATTTATAATTAAAAAAATTAAA
ATACACGCCGTTATTAAATTAATTTGAAAGTTTAGAGACTAAATAAAAGGGAAAAA
AAAAGGAAAGTTGGAACGATTAATTGTCAACCACGTAAAAAGGACCTGATAGGAAT
AATTCTTCAATGACACACTCTCCCCCTCTTCTTTTGTTATAATTTCGCTTTCATTTCAC
TCACACTCTCACATCATCCAACCAACCAACACAACACACACCACCATTTTTTTTCAA
TTTGGAGCCAAAAAAAGAAAAACGGAAATTGTTTGGTAAGAGAGATGAAAAAATG
GTGTTCAAAAGGAAGCCAAATTAGGAGCGAAGAGTATGGAAGAGGAGACGTAGAT
TGGGAGCTCCGACCAGGTGGAATGATTGTTCAGAAACGACATGTCGGGTCGGGTTC
GGGTTCAAATTCGGAGCGTTTCATTACAATCAACGTATCTCATGGGTCTTATCGTCAT
CAAATCACCGTCGATTCTCATTCCACATTTGGTATGTTATCATTTCAATTTGGGGGTT
TTTTTGAAATACAGATTGATTTTTATTTTAAATTGAAGACTGAATTATTAATTTTTGT
GTTTGGGACAGGGAATTTAAAGACAGTTTTACGACAGCAGACAGGGTTAGAGCCGA
GGGAACAGAGATTGTTGTTTAAAGGGAAGGAGAAGGAGAACGACGAGTGGTTGCAT
ATGGCCGGTGTGAACGACATGTCGAAACTCATACTCATGGAAGATCCTGCTACTAAA
GAGAGGAAGCTTCAAGAGATGAAGAAGAAGAATACCACTGCTGCAGGCGAAGCAC
TGGCGGGGATCAGAGCGGAGGTCGATAAACTCTCCGAAAAGGTTCGTTAAATCGTT
AAATTACAACTTTAGTCGAAAAATATATTTGAGAAAATTGTAAAAACCACTCGTGGT
AATTACAGTTATACCTTCAAACTTTTAATATTAAAAATTAAGTCTTTAAATTTATATT
ATTGTTAAAATTGGACTCTTAAATTTTGTTTAATTGTAGAATTGAAGCTCAAAATGAT
AAAAATTGAACTCTCAAACTTATACAATTTTTACCATTTCTATTATTACTTAAGTTTG
AGGTCTCAATTTTACCATAAAAAAATTTAAGAGGTGGAATTGCAAGTTATAACTATT
ATCATACTTTAAGGTCAATTTTTACCATTTGTCTAGGATATTTTTTGGTTGGTATGGT
TTATGTTTTTAAATTCTTAATTTCTCTAACATTTTGTGTTTAATAAATGGGTAATAATT
TATTTCATAAATTTTTTAAGTTCACACCTAAAATTCAATTTTATAACTAAAAAATTAA
TTAAATTTTACTTATTTATTTATTATGATATTCACATACTTTTAAGATATTTGAATTCT
CAAGTGAATTTTTTTTTAAACAACAAGTTTTTCTGGAAATTGACAAAAAAAAGAAAA
AAGTAGTTTTAAATGCCTTGCTTTTATTTTATTTTATTTAATGAAGTTTTGATAATGAT
ACAAATGTTTATGTAACAAAAATGAAAACATTTGAAAGAAAAAAATGGTTATTAGG
TAACATTTCTAAAGTTTAAAAACCTATTTGAGACAAATGTGAAAGTTCAATAACTCA
TTGAATTGCTTTAGAAGGTTTAAAAAACAAATAGTTACAAACAAGCTTAGAGACTA
AACTTCTAATTGAACCTAATTCTAAATGATTGAAATGAATTGACCAATGGATAACTA
GCATATTATACATTTTTTTAAAAAAAAAAATTGGAAAGGGGCAAAAAAGTGTGAGT
GTATAAAATTAGGGTTTTTAAGTGCAGGTTGCGGCAGTGGAAGGTAGTGTTAATGAC
GGGAAGAGGGTGGAAGAGAAAGAAGTTAATTTATTGATAGAGTTGTTGATGATGCA
ATTGTTGAAATTAGATGCAATTGAGACGGATGGGGATTCCAAACTTCAAAGACGAA
CTCAGGTATCTATTGGACTATATGTCAATTATCATTAAAAATAAATTTACTTTGGCTT
ATCTATTTATAATAATTAGGGTATACAAATTTAAGGTGATTTAAATCGTTTTCTTTCA
TTAATCTAACTATGCAAAACCGTTACAACAATACGTCACCTTTAAATAACTTTAACT
ATTTACCAAAACTTTATGAAGAGGAATTATAAATTTACTTACCGCCTAATTTCTCTTT
TAAAACTCTTTTTGTTAACTCTTAATGTCGGGTATGTTTGCATTAGTCATATTTAATA
TCCATTAAATGATATAACTTTTCAAACAATAATAATTAACATATATCTTTATTATTAT
TATTAGTTATTAGATTTGTATAGTTTTCTAAAAAAAAGAATGGATTTTATGTAAGTTT
GGATTAACTTAAAAAATAAATATTTTTTTAAAAATTATTTTTATTTAAATTTTTGTGA
CAAAAACTTTTTAAAATAAAAACACTAAATTCCATTTGGATGAATTATATATTTAAA
TATTATATTTCTATGGTAGAAAATTATTTACTATTATTTAATCAATTTTAATAATGAT
GGATTAAATTTAAGTTTTATTAAATGAATACTTGAAAATATGAATTAAAATTAAATA
TATATATTTTAATTTTTCAATTTTGGTATTTATAATAAAAAGTACGATAGTTTAATCA
TTAATGGGTTAGGTGGCTGGTGCTCCCCTAGGGCCATCAAACTTAAAATAATTAAAA
ATAATGAAAGTCTCCTAAATTGTATGAAAATTCAATGAATATAAATTGTGAAAAATG
ATAATGGGTATTTTATCTATTTATTTATTAACTCAAAAAAAAAAATTAATAAATATA
GACTAAAAAAATTGCAGAAATAGGACAAAATGATTTTAATTCTTTCCCTTGATATGA
CATTTTTATGTGGGACATTATGAAACCAAGAACTTATCAAGAAGGATTCTATTCAAA
ATAAATAAATAATTGATTAAAGAAGAAAATTCCATTAATGTCCCTAAAGTCTTAATC
ACACCTCTATTTAGCGTCTATCATGAATAAAATAAATAGAAATCATAGGAATGCTGA
GGTGGCATGAACACTAGATAAAAATTTTAGGTTTAAATACTACTTTAGTTTTTATATT
TTTCACATTGTTTCATTTAGATTCACGACCTTTTAATTTTGGTTAAATTATAAATTTAG
TCCATATAATTTGAAGAAAGTTAAAATTTAATCCTATAGTTTATAATTAGAATTTAAT
CTCTATGATCTGATAAAATCCTCATAAATAATCTCACTACTGTAGAGACTAAACTAT
AGGGAACATTTATAAGGTTTTATCAAACCATAGGAACTAATTCTAGATTTTAAAACC
AAATGGACCAGATTTTAATTTTCTCCAAACTACAGGGGCCAAATTCTAATTTTTTCTA
AATTATAGGAGACAAATTTGCAATTTAACCTTTAACTATAGTTAATTTTGGTCCACTT
ACTTTCAAAATATCAATTTTAGTCCCGTGGTTTTAAAAAGTCTCCATTTTGGCCCCTT
AACAATGAACAAAAATAAGATAAAAATAGTAATTAAATTTTAATTTTGAACTATGTA
ATTTTTTTTTTGAAGTACAAATAGTAGAGTAGGGAAATTGAGAGAAAGAGTATACGT
TAATTATCATTGAACTATGTTTATTTTGGTGGTGATAAGTTTTTACGCAATTTCAATT
AATTTGAATAACGTTAGAATTGTAATTTTATAATTTTGGGAATAAAACAGGTTGTTA
GGGTACAGAAATTAGTGGACAGAATTGACAAGTTGAAGGTTAGAATCTCAAATCCT
TTAAACCAAACAACAATGAAAAGAGGCAAATGGGAGGAATTTGAATCTGGATTTGG
CAGCCTTATTCCTCCAACTTCAAAACTCACCATCAGCTCTACAAAAATAACTCATGA
TTGGGAACTCTTTGATTAGTTCATTCTCTTTCTTCCCATTTTTTTGCATTAGAACCGAA
CCGAATCGAATTAAACTATTTTGGCATTTCTGTACATATTGCTTTATGTGGGCTTCCC
AATTGATATTGGACCCAAATGGGCTCTGTTATAAGCCCAATAAGATGTCTGTGCAGT
GTGATGTTGGGTTAAGTGGAATATTATTACTCTCTCTTTTTATCAAATTCTTTCGCTTT
TTTTTTTTTTTTAACCATTGTCAACGAGTAATAATTTAGTATCTAATATTTATTATTTT
TTAAATATTTAGGCTATATTTAATAATAATTTTGTTTTTGAAAATAAAGAATATTCTT
TCCCATTTTTTATTAGTCAAATTCCAAAAACAACAAGTTTTTAAAAGTTACTGTTTTT
AGTTTTCAAATTTTGGCTTGGTTTTTTAAATCATTAGTAAAAATTAGATAACAAAAG
AATAAATTTTGAGATGGAAGTAGTGTCTATAGACTTCATTTTCAAAATCGAAAAAAA
AAAAAAAATGGTTACCAAATAAGGCCTTAGTTTTTGTGCTTTTATCTTCATAATAATT
TAGATCAAATTTGGTAACTATTTGGTTTTGGGTTTTTAATTGAAAATTAAGCTTATAA
ACATCCTTTTTATCTTTAAATTTCTTGTTTTGTTATCTACTTTCCACCAACATTTTAAA
AAATAAAGTTATTTTTTGAAAAGTAAAAGGAAATAATTTTTAAAACTTATTTTTGTTT
GTAAAATTTAGCTAAAACTCATTTACTTCATAAATGTATTGAAAATCATAGTAAAAA
ATTACGAGAAAATATGTTTAATTTTCAAAAACGAAAAATCAAATAGTTTTTAATAGT
TCCTTAGCCTCTTTATTTTTATTTTTTTAATACCATATCTCTAAATTTTAGTATGTAAC
CATTTAGTATTCTTTTAAACTTTGAATTTTTAAACAAGTTATTATTCATTAGATTCTA
AAGAAATTTCTTCTATATGTAAATAGATAAAACAATTTAATGATGAAATGCATTTAT
AAATTACAATTTATTAGATTGTTACAAAAAAAAAAAAAAGAAAAGAAAAAAAAATA
GTTCACACTTGCTTGCAATGGAATTTTTATATCAGGGTATGGATGTAAATTGTTACA
AACACAGTTAATCATTGTTTGCCTTTAATATTTTCAATTATACATAGAAAGTTGGCTC
ATACATTACCTTTCTCAAACATGTTATTTATGGCAACTTCTTAGTTTACTCTCTTCCTC
TCTATTTCTTTGCCTTTCCTCTACCATTAAGACTCCTCTTGTTATTTTCAAAGACTATT
TAATTTAATTAAATAACGCTAATGAGTTTTAATAATTATCTAATTAATATTATAACGT
TTTCGTTTTACTGATCTCTTAATTTTAGAAGAATAAGGACTTCAATCAATAGTTATAT
ATTTGTTAAAAATCTATTGATCCAATCTTTTATAAATAAAACAAGTCCAAAATTAAA
CAAGAAAGATCGATGATATTATCGAACACTTTGAAATAATTATGAACTTTTTAATAA
GTTAATGTAGATATGTTTTAAATATAAGAAGGGCCATGCTTTACATGGTATCAACTT
TAAGTCTCATGCAAATATTGCAACTCATGGGTGTACAAAGATAGATCAACAAGCAT
ATTTATCAATTTTTTAAATTTAAAAACAAAGTTCATTTCTTTAATTTTCATAATCATA
GGGTTTATAAAAAGGCTACATAGTCCCTACCAATTCATTCATTATTTCTTCCCTTTGG
CTAAGGTACGTACATACATTTAACTATATAAAATGATTTTTTTTCAAAACTATGATAT
ACATAACCATATATATATTTTTTGCCATTTTGTTTTTTAGGCATCTTCTAATCAATCTC
ATGGAGGAAAGTTACAAAATGAGGATGAACCAAGGCGGAGGAATTCCGACGATGG
CAGCAGCACTGCCACCATTGCCACCGTCATGTTTAGGAAAACTGACAACTAGCGGT
GAAAAAAAGCTACCGTTCTTTCAATCCAACATGAATTTAAGTATGTATGGAAATGAC
AAAAGTATCCTTTCTCAAAGAGAAGCTACCATAACACCACCGCCGAAGCAACACCA
ATCACAACTTCTAGACTCCGACAAAGATCTCACTGTCGAAGCCAAGCGACTAAGAA
GGTTCACTCTCTTGAAACTAATCTAGAACTTAAAAGTAAAATTTAAAAAGTCATTTC
AAACAACCCTAACGTTCTTTTATTCATGTACGTACGTAAATACGTACATAAATGTTTT
TTTTTTTTTTTTTACATGGGTTAATTTTTTATTTTTTACTATAGGGTGATGCAAAGCAG
ACAATACTCTCAAAAGTATCGACTAAAACAGCTTCATTATATTACTCAGCTTGAATC
AGAACTAAAAGCCCTTCAAGTAATTACATAAATCAATAAATAATATCAATCAATTAA
TTAATTAATTAAATTAGTTGATTAATCACCTTTTTTGGTTGAACGAATTAAAACAGGC
AGAAGTAACAATTACCACACCGAGGATAAAATTCATGGACCGTCAAAATTCACTGC
TCCGAGCCGAAAATTACTCCATCAAAGAGAAATTATCTGCATACACCGGAGAACTTC
TATTCAAAGAAGGTAAATTAAATTAAAATTTTTATTTTATTTTTAAAAATTTTAATAT
GATATTTTTGCATGGGGCAGCTCAATACGAAGAATTGAAAAGAGAGAGAAATATGC
TTAAGGAAATCTACGAAGCATATCAGTTAAAATTGCTGGAGACTCTGAAAAGCAGC
AACAACAACAACAACACAACTGCTGCCAGTGGAAGCACTTTTCAATTGGTCGAAAA
TTACCCACAAATTGCTACCAAATCAAACCCATTTACCATGCTCGAAAATTAAATTAA
ATTATCAAAATCAACAATAAGACATTTTGAAATTTGTAATAGTTAATTAAACCATGG
TGAGATGGATTTAGTTGTTGTCATTTCTAAGATATATATGTGTGTGTGTGTATGTTTG
AAATATATTTTTCCTATGTAATTTGTAGGGTTGAATTTGAAGGGAACTTTTGTTCAAT
TTGTAATTCAATTTCTGATTTTTCTTTCTATATTAAATTATGTCACATTTTAATGTTTA
GGGCTGGCATGAGATGATGAAAGGCTATATATGCATGTATATTCATAAATTTGTTTC
CTTAAGAAATTTGATGATGCTGGACATTGGATAAGACTAATTGGCAGCCTGATCATA
TGCTTCAATCAATATTTCTATAATGGAATAAGCAAATTGGTAAGTGTGTGGCCTCCT
GGCATGGTTGTGGACGTGATTGGCGAAATCAAAAGGTGGGGAGACACATCTCATAC
TCCATTTGCCAAAGGTTGAGCAATTAGCTCGTTACTCACTGCCTTACCCATCAACCAT
GCTTTGGTGTGAGCTTTCAGCTTTCAGCTTTCAGCTTTGTTATTTACAATATATATTTC
CTCTCTTTCAACTGCTCCATCTTCTTCTGTGATCCTTACTTTCCTTTATGATTGTATAA
TGAGAATGTTTGGAAAATCGTAATAAGATAGACTTGTAATGTAATGTAATCCAAAAT
TAATGTTTGGATTGAACGTTTTGGACCCGATTTGTAATACGAAAGTCATTCTGTTCCG
ACAGTTGTCAACCCAATCCTATTTTTCATCGTTTTCATGGTTCACAATCTCATTTTCGT
TTATTTCTTAAATACTCACACATTCCAATACTCCACTAATTTCCAATAATCCTATTAC
ATTCCACCTCCCCCTTAATTCTCTTTAACATACTAGTTTGCACATAGTTAAAAAATTT
TAGAATATAAAATTTTAAATGAATATGCTTTTATTTAAATTTAAATAGTAAATTTTTT
ACGTGAGATTTAAAAGTAATGAAAAATATAATATATATGTATATATATATATATATA
TAATTTGGTTAGTTTTTTAGAAGGGAAAACAAAATTGTTTAGATACATAAAAAGTTA
AAACATATGATTCTTACACCGTACTAATTTTCTAAACAACCAAAAAGGAATCCAAAA
CTTTATATTAAAATATAAAAATCTTCAAAATTTCCGCTTAAAACTCAGCAAAACAAT
TAATAGCAAAATATAAAAAAAATGCCTACCAAAAAATATCATATTTGATCCTGATA
ATTTTTTTAATTGATCATAGCAAGCAAACTAATTTAAATTGTAAAAATGATCAACAA
AGTCTCCTCATCGAAAAGTGTTGGGTCATCTATTAAATTAGAGGGAGAGAGGAAAT
AAAAGATTGAGGTGAAATGGGAGGGTAGATAGCAGCTTTCATCTATATACTATGCT
AAGGACATATTTTAATTTTTCTTTAATGTTTAAGTGCATTATTGAAACTTTAAAAGTT
TCAGAGGTATTTTTTTTAAAAAAAAAAATTAGTAGTTTTTGTTAAAAACTAACAACA
ATCGTATTTTTGAAATTTTTTTTAAACTTAAAGTTATTAATGAAATTTTGGAAAGTTT
ATGGTTATTTTTAAAATAAAAAAATTGTATATCTTCTATAATTTATCTTGAATTTTCT
TTGTTTACAAATTTGATTTTACATGTTTCAAGTAGCATTTTTAAGTGGCATGGTCAAA
TGTTTAAAATATACCATGCAAATGAAAGTTGTTTAATTTAGCTAAATTAAACTTATC
AAAATCAAACGTTATTTAAATTTCACTATTCTTTTTATAATATGTGTGATAGGAAAAT
AGAACTTCTCACCAAAATGTTGATGTACAAATTTGATGAGTTTGAAAAATTTAACTA
ATTACAACTAATGGTAAGATTCAACTTCTTACCCTAGCTTCTTACTTCTTTGAAAGTA
TGAAATTATATACATAAAAAGACAAACTAATTTGCTAAGTCTTCCAAAATAAACCAT
AATATTTTAATTTATTTCATCTCAATTTA
>CL08381_WT_allele
SEQ ID NO: 14
MACCAATGTCGGCGGCTGATGACGTCCGTCACGAAATGCATCATARAGTG
ACGAACTTTTATCCGTGTAGATTTTTGGTATTTCCATCCCTTGCGGAGCCGTCATAAT
TCCAAAACGGCAATGCAAAATCAGGATCCTTAATCAAAGACCCCAATATTCTCTCAT
GAAAGTAAAGATAAAAACGATGGAATGGGAAGAAC
>CL08381_MUT_allele
SEQ ID NO: 15
MACCAATGTCGGCGGCTGATGACGTCCGTCACGAAATGCATCATARAGTG
ACGAACTTTTATCCGTGTAGATTTTTGGTATTTCCATCCCTTGCGGAGCC [symbol] GTCATAA
TTCCAAAACGGCAATGCAAAATCAGGATCCTTAATCAAAGACCCCAATATTCTCTCA
TGAAAGTAAAGATAAAAACGATGGAATGGGAAGAAC
>CL_chr2_gap_F1_primer
SEQ ID NO: 16
AGAGTGAACCAAAAGATCC
>CL_chr2_gap_R3_primer
SEQ ID NO: 17
CCCAAAACCAAATAGTTACC
>CL_chr2_gap_F2 primer
SEQ ID NO: 18
GAACCAAAAGATCCACCA
>CL_chr2_gap_R1 primer
SEQ ID NO: 19
ACCTACATCCACTCCCTAA
> PPO_WT_gDNA reverse complementary sequence
SEQ ID NO: 20
TTATGCATCATATTCAATTCTAATGTCCTTAACGGTGGCGGACCCATCTCC
AAACCTAGGGACCAATGTAACAATAATGCTATCGTCGTTATCCGCATCCAAACTCTC
AAGCAGTTCAGTTATCCCTAACCTAAGGCATGTTTTTATGTTCATGCTGCTGCTACCT
TTCATATGAGGCACATTCACAAAGCTCCCTGCAAACTCAGAATTATCCGCTCTAATT
TCCCTATCATCCTCGTCATTGATAAAAACATCAAACTTAATAGCCTTGTTTCCGTCGA
ACTCAATCCCATCAATCACCAAAATCTCCTCTTCATCGTCTTTCTCCTTCGTACCCCT
CGATTTCTTCGGCCTCTTGACTTCGAAGCTGACGATCTTGTCAACACTCGACGGTAG
CTTCCCGGTCTTCTTGGTAGATTTCTTCTTGGTTTTGTTGGGTGTGCGTGGTACTCGTG
GGGTTGGAGGTGTTTTGAGCCATGGAATTGGTACGGTGTCATCGTAGACATAGCCTA
AGGCTCTGGTATCTAGACAGTCTTTGACATAAACTCGGACAGCTTCACCATTCTCAT
CGTAGAATACAAAGGAAGCGTTTAGGAAATCTTTGTCTTTAATGTCTTGTCGCTTTTC
GCCTAAGGATTTCCATATGGACCAAAAACGGTCCACGTTCGCGTGGTGGGCGTAGA
AGATGGGATCTCTAGCCGCTGAGAAGAAGGTTCCCATGTCAATTCGGTTCGATTGGT
TCGGGTCACCCGTCCACAAATGAATTGAATTGTGAGGAAGGTTTTCCACCGTCCCCA
TTCCTTTCAAAAAATTAATCAATTCATCAAAATTACATCATTTTACTTTCCATACTAA
TATCCTTAATTATTATCTCTAAACATCATTATCTAATTTACAAATGGGCATGTTTAGA
ATACATTTTCAAATGATTAAATTAAAAAAACAAGTCAATTTGATGACCAGCATAACT
TATCAACACAACCCAATCATCACTGTTTCGATTGGGTTAGGTTGAGTTCAAATAAAT
TAAAAAGTTATTGGTTGAGTTGTTTATGTGTTCTCTTCAAATAACCCCAACTTGTTAT
AATTCTTTTATAAAAGATTTACTTATGGTACAATTATATATACATATATTTATTTTAA
TTTTATTTAATTTCATAATTTTTTGGAAAGTTTATTCTCCAGAAACTTCAAACAATAA
TATTTTAGCATTTTGAAAAGAAAATCTTCATAATATAAATTGAAATTGAGTTGTTAA
TTTTAATTCAATATATAAAAATAATTCAACAAAAATTTTACATATTAACATTTTAGG
GTTGTTTTCAAATATAGCAAAAAAAGTCAAATTATTTGCAAATATTTCATTGTCTATC
CGTGATAAACCGCGATAAACTTTTATCATTAGGGGATAGACTCAATTAAATCTTTCT
GTATTTGTAAAAACTTTGATTTTTTTCCATTTATAATAGTTTCCTAACATTTTACTTAT
TTTTAGAACAGAAATTTACATAAATGACCTAAGTAATTTGAACCCATATATTTCATG
ATTGAGTTGAATTCGATTCATTATTTAATAGAATTTATTTGAATTGAAAAAACTTATC
AATCCGACAATTGAGTTAGATCTATAAACTGCTCTAATCCAACCCAAAAACACTCTT
AACATTAATTTTCAAAAACTAAAAACATTACCTAACATAACCATAACTATATATGAT
TGTCTTTTTTATCTTTTCCTTTTTTTGAAAAAAGAAATTATATGGTTGAAAAAAAAAA
TCACCTGGACTTGGGTTGCTGCCACTTCGATAAGGCTGGCCGAAAAAGAGCAAGGG
CGTACGGGCGCCGGACACGACCTGGCGATACATAACACTTAGATTGCATTGGATTAT
CTTTTCTCTGCTTATTGTTGGCTCAACGTCATTGTAATCCAAATCAACCAATGTCGGC
GGCTGATGACGTCCGTCACGAAATGCATCATAGAGTGACGAACTTTTATCCGTGTAG
ATTTTTGGTATTTCCATCCCTTGCGGAGCGTCATAATTCCAAAACGGCAATGCAAAA
TCAGGATCCTTAATCAAAGACCCCAATATTCTCTCATGAAAGTAAAGATAAAAACG
ATGGAATGGGAAGAACAGCCACGAGAAATGAACTTGTAATTCAACTGGAAGACCCA
ATTGATCGTAACCCCCAGTACAATAAGCACAGTGAACAAGTGCTTGCTGTTTAAAAC
TACGTGGATCATCATCAGGAAGCGCTTTCATAAGCGCTACGGCTTCCTTATACTTTTC
AATATATTCTTTATCTAATGATTGTGCCGCTTTCCTAACGCGTGGTTTGAGGAAGGGT
TTTACGTTATTGGTGGATGGTGGGCAGCAAACCAAATCTTTGACGCCATCTGCCAAG
TCCGTGCTTGATCCACACTTGGAAGGGTCGGGGGTTGTGACTGGAGCTGCCAAAGCG
AAGGGATCAACTCCAAAAGCACTTGAAGCTGAGCCATACAGACCGCCGAGCCCGAT
AAGCGCTTCTCTCCGGTCAACAAACTTGCCTGGCCATAATGAGTTATTACTTTCTTCA
CCACTGCCATTGGAGCCGCTACACACAACCAAGTTATTGAGTCTATGAATGGTGGAA
GATGGATCTTTTTTTTTACGATAAAACAGACCAAAGGAGGCGCCGCCGGTGGTGGCC
GTGGTTATTGCGGCGGAGGAAAGTGCTAGTGGCATGGAAGGAGATAGAGAGGCCAT

The invention is further described by the following numbered paragraphs:

1. A modified watermelon POLYPHENOL OXIDASE (PPO) gene, the wild type of which is identified as SEQ ID NO: 1, encoding the protein of SEQ ID NO: 5, or the wild type of which encodes a protein that has at least 90% sequence identity to SEQ ID NO: 5, wherein the modified PPO gene comprises one or more nucleotides replaced, inserted and/or deleted relative to the wild type, and wherein said one or more replaced, inserted and/or deleted nucleotides result in an absence of functional PPO protein.

2. The modified PPO gene of paragraph 1, wherein the modified PPO gene confers a pale seed color to a watermelon plant when present homozygously.

3. The modified PPO gene of paragraph 1 or 2, wherein the modified PPO gene, which when homozygously present in a watermelon plant causes the production of pale seeds, comprises a premature stop codon that leads to an absence of functional PPO protein.

4. The modified PPO gene of any of the paragraphs 1 to 3 wherein one or more nucleotides are replaced, inserted and/or deleted relative to the wild type gene at position 1 to 712 of SEQ ID NO: 1 resulting in a premature stop codon, which modified PPO gene when homozygously present in a watermelon plant causes the production of pale seeds.

5. The modified PPO gene of paragraph 4 wherein the modified PPO gene comprises an insertion of a T between nucleotides 711 and 712 of SEQ ID NO: 1.

6. A watermelon plant comprising the modified PPO gene of any of the paragraphs 1 to 5, wherein the homozygous presence of the modified PPO gene causes the production of pale seeds.

7. The watermelon plant of paragraph 6, wherein the modified PPO gene that confers a pale seed color to the plant when present homozygously is as comprised in the genome of a Citrullus lanatus var. lanatus plant representative seed of which was deposited under accession number NCIMB 43364.

8. The watermelon plant of paragraph 6 or 7, wherein the modified PPO gene is homozygously present and the plant produces seeds with a pale seed color.

9. The watermelon plant of any of the paragraphs 6 to 8, wherein the plant comprises a non-functional HLS1 gene, the wild type of which HLS1 gene is identified as SEQ ID NO: 7 encoding the protein of SEQ ID NO: 9, or the wild type of which HLS1 gene encodes a protein that has at least 90% sequence identity to SEQ ID NO: 9, and/or the plant comprises a non-functional BAG4 gene, the wild type of which BAG4 gene is identified as SEQ ID NO: 10 encoding the protein of SEQ ID NO: 12, or the wild type of which BAG4 gene encodes a protein that has at least 90% sequence identity to SEQ ID NO:12, wherein the absence of functional HLS1 protein and/or the absence of functional BAG4 protein confers a microseed size to the plant.

10. The watermelon plant of paragraph 9, wherein the non-functional HLS1 gene comprises one or more nucleotides replaced, inserted and/or deleted relative to the wild type, and wherein said one or more replaced, inserted and/or deleted nucleotides result in an absence of functional HLS1 protein, and/or wherein the non-functional BAG4 gene comprises one or more nucleotides replaced, inserted and/or deleted relative to the wild type, and wherein said one or more replaced, inserted and/or deleted nucleotides result in an absence of functional BAG4 protein.

11. The watermelon plant of paragraph 9, wherein the HLS1 gene and/or the BAG4 gene is non-functional because it is absent from the genome.

12. The watermelon plant of any of the paragraphs 9 to 11, wherein the non-functional HLS1 gene is homozygously present and/or the non-functional BAG4 gene is homozygously present or the HLS1 gene and/or the BAG4 gene are homozygously absent resulting in the plant producing seeds with a microseed size.

13. The watermelon plant of any of the paragraphs 6 to 12, wherein the plant comprises a deletion on chromosome 2 corresponding to 13962 bp being deleted between base pair position 29902114 and 29916077 on the Citrullus lanatus 97103_v1 genome, wherein said deletion confers a microseed size to the plant when present homozygously.

14. The watermelon plant of any of the paragraphs 6 to 13, wherein the plant comprises a deletion on chromosome 2, wherein said deletion is as comprised in the genome of a Citrullus lanatus var. lanatus plant representative seed of which was deposited under accession number NCIMB 43364, and wherein said deletion confers a microseed size to the plant when present homozygously.

15. The watermelon plant of paragraph 13 or 14, wherein the deletion is present homozygously and the plant produces seeds with a microseed size.

16. A watermelon seed, comprising the modified PPO gene of any of the paragraphs 1 to 5, wherein the plant grown from said seed produces seeds with a pale seed color as a result of the homozygous presence of the modified PPO gene, and optionally further comprising the non-functional HLS1 gene as in any of the paragraphs 9 to 15, and/or further comprising the non-functional BAG4 gene as in any of the paragraphs 9 to 15, wherein the absence of functional HLS1 protein and/or the absence of functional BAG4 protein confers a microseed size to the plant grown from said seed.

17. A watermelon fruit produced by the watermelon plant of any of the paragraphs 6 to 15, wherein the watermelon fruit has seeds that have a pale seed color and optionally a microseed size.

18. Food product, comprising the watermelon fruit of paragraph 17, or a part thereof, optionally in processed form.

19. Propagation material capable of developing into and/or being derived from a plant of any of the paragraphs 6 to 15, wherein the propagation material comprises the modified PPO gene of any of the paragraphs 1 to 5, and optionally the non-functional HLS1 gene and/or the non-functional BAG4 gene as defined in any of the paragraphs paragraph 9 to 15, and wherein the propagation material is selected from the group consisting of a microspore, a pollen, an ovary, an ovule, an embryo, an embryo sac, an egg cell, a cutting, a root, a root tip, a hypocotyl, a cotyledon, a stem, a leave, a flower, an anther, a seed, a meristematic cell, a protoplast and a cell, or a tissue culture thereof.

20. Use of the modified PPO gene of any of the paragraphs 1 to 5 for producing a plant that produces seeds with a pale seed color.

21. Use of paragraph 20, wherein the plant that produces seeds with a pale seed color is produced by introducing the modified PPO gene into its genome, in particular by means of mutagenesis or introgression, or combinations thereof.

22. Use of the plant of any of the paragraphs 6 to 15 for the production of a watermelon fruit having seeds that have a pale seed color and optionally a microseed size.

23. Marker for the identification of a modified PPO gene, wherein the marker sequence detects an insertion of a T between nucleotides 711 and 712 of SEQ ID NO:1.

24. Use of the marker of paragraph 23 for identification and/or selection of a watermelon plant that produces seeds with a pale seed color.

25. Method for selecting a watermelon plant that produces seeds with a pale seed color, comprising identifying the presence of a modification in the PPO gene, optionally checking the color of the seeds the plant produces, and selecting a plant that homozygously comprises said modification as a plant that produces seeds with a pale seed color.

26. Method of paragraph 25, wherein the identification is performed by using the marker as defined in paragraph 23.

27. Marker for the identification of a deletion on chromosome 2, wherein the marker sequence detects the presence or absence of a deletion corresponding to 13962 bp being deleted between base pair position 4930 and 18893 of SEQ ID NO: 13.

28. Use of the molecular marker of paragraph 27 for identification and/or selection of a watermelon plant producing seeds with a microseed size.

29. Method for selecting a watermelon plant that produces seeds with a microseed size, comprising identifying the presence of the deletion on chromosome 2 using the marker of paragraph 27, and selecting a plant that homozygously comprises said deletion as a plant that produces seeds with a microseed size.

30. Method for producing a watermelon plant that produces seeds that have a pale seed color, comprising modifying the PPO gene of SEQ ID NO:1, wherein the modification results in an absence of functional PPO protein, and the absence of functional PPO protein leads to the seeds of the produced plant having a pale seed color.

31. The method of paragraph 30, wherein the plant in which the PPO gene is modified has seeds with a microseed size.

32. Method for producing a watermelon plant that produces seeds that have a microseed size, comprising modifying the HLS1 gene of SEQ ID NO: 7 and/or the BAG4 gene of SEQ ID NO: 10, wherein the modification results in an absence of functional HLS1 protein and/or an absence of functional BAG4 protein in the plant, which leads to the seeds produced by said plant having a microseed size.

33. The method of paragraph 32, wherein the plant in which the HLS1 gene and/or the BAG4 gene is modified has seeds with a pale seed color.

34. A modified nucleic acid molecule, the wild type of which is identified as SEQ ID NO: 13, or the wild type of which that has at least 90% sequence identity to SEQ ID NO: 13, wherein the modified nucleic acid does not comprise SEQ ID NO: 7 and/or SEQ ID NO: 10, wherein the modified nucleic acid confers a microseed size to the watermelon plant when present homozygously.

35. The nucleic acid molecule of paragraph 34, comprising a deletion corresponding to 13962 bp being deleted between base pair position 4930 and 18893 of SEQ ID NO: 13.

36. Use of the modified nucleic acid molecule of paragraph 34 or 35 for producing a watermelon plant that produces seeds with a microseed size.

37. Use of paragraph 36, wherein the watermelon plant that produces seeds with a microseed size is produced by introduction of the modified nucleic acid molecule into its genome, in particular by means of mutagenesis or introgression, or combinations thereof.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A modified watermelon POLYPHENOL OXIDASE (PPO) gene, the wild type of which is SEQ ID NO: 1, encoding the protein of SEQ ID NO: 5, or the wild type of which encodes a protein that has at least 90% sequence identity to SEQ ID NO: 5, wherein the modified PPO gene comprises one or more nucleotides replaced, inserted and/or deleted relative to the wild type, and wherein said one or more replaced, inserted and/or deleted nucleotides result in an absence of functional PPO protein.

2. The modified PPO gene of claim 1, wherein the modified PPO gene confers a pale seed color to a watermelon plant when present homozygously.

3. The modified PPO gene of claim 1, wherein the modified PPO gene, which when homozygously present in a watermelon plant causes the production of pale seeds, comprises a premature stop codon that leads to an absence of functional PPO protein.

4. The modified PPO gene of claim 1, wherein one or more nucleotides are replaced, inserted and/or deleted relative to the wild type gene at position 1 to 712 of SEQ ID NO: 1 resulting in a premature stop codon, which modified PPO gene when homozygously present in a watermelon plant causes the production of pale seeds.

5. The modified PPO gene of claim 4, wherein the modified PPO gene comprises an insertion of a T between nucleotides 711 and 712 of SEQ ID NO: 1.

6. A watermelon plant comprising the modified PPO gene of claim 1, wherein the homozygous presence of the modified PPO gene causes the production of pale seeds.

7. The watermelon plant of claim 6, wherein the modified PPO gene that confers a pale seed color to the plant when present homozygously is as comprised in the genome of a Citrullus lanatus var. lanatus plant representative seed of which was deposited under accession number NCIMB 43364.

8. The watermelon plant of claim 6, wherein the modified PPO gene is homozygously present and the plant produces seeds with a pale seed color.

9. The watermelon plant of claim 6, wherein the plant comprises a non-functional HLS1 gene, the wild type of which HLS1 gene is identified as SEQ ID NO: 7 encoding the protein of SEQ ID NO: 9, or the wild type of which HLS1 gene encodes a protein that has at least 90% sequence identity to SEQ ID NO: 9, and/or the plant comprises a non-functional BAG4 gene, the wild type of which BAG4 gene is identified as SEQ ID NO: 10 encoding the protein of SEQ ID NO: 12, or the wild type of which BAG4 gene encodes a protein that has at least 90% sequence identity to SEQ ID NO:12, wherein the absence of functional HLS1 protein and/or the absence of functional BAG4 protein confers a microseed size to the plant.

10. The watermelon plant of claim 9, wherein the non-functional HLS1 gene comprises one or more nucleotides replaced, inserted and/or deleted relative to the wild type, and wherein said one or more replaced, inserted and/or deleted nucleotides result in an absence of functional HLS1 protein, and/or wherein the non-functional BAG4 gene comprises one or more nucleotides replaced, inserted and/or deleted relative to the wild type, and wherein said one or more replaced, inserted and/or deleted nucleotides result in an absence of functional BAG4 protein.

11. The watermelon plant of claim 9, wherein the HLS1 gene and/or the BAG4 gene is non-functional because of its absence from the genome.

12. The watermelon plant of claim 9 wherein the non-functional HLS1 gene is homozygously present and/or the non-functional BAG4 gene is homozygously present or the HLS1 gene and/or the BAG4 gene are homozygously absent resulting in the plant producing seeds with a microseed size.

13. The watermelon plant of claim 6, wherein the plant comprises a deletion on chromosome 2 corresponding to 13962 bp being deleted between base pair position 29902114 and 29916077 on the Citrullus lanatus 97103_v1 genome, wherein said deletion confers a microseed size to the plant when present homozygously.

14. The watermelon plant of claim 6, wherein the plant comprises a deletion on chromosome 2, wherein said deletion is as comprised in the genome of a Citrullus lanatus var. lanatus plant representative seed of which was deposited under accession number NCIMB 43364, and wherein said deletion confers a microseed size to the plant when present homozygously.

15. The watermelon plant of claim 13, wherein the deletion is present homozygously and the plant produces seeds with a microseed size.

16. The watermelon plant of claim 14, wherein the deletion is present homozygously and the plant produces seeds with a microseed size.

17. A watermelon seed, comprising the modified PPO gene of claim 1, wherein the plant grown from said seed produces seeds with a pale seed color as a result of the homozygous presence of the modified PPO gene.

18. The watermelon seed of claim 17 further comprising a non-functional HLS1 gene, the wild type of which HLS1 gene is identified as SEQ ID NO: 7 encoding the protein of SEQ ID NO: 9, or the wild type of which HLS1 gene encodes a protein that has at least 90% sequence identity to SEQ ID NO: 9, and/or the seed further comprises a non-functional BAG4 gene, the wild type of which BAG4 gene is identified as SEQ ID NO: 10 encoding the protein of SEQ ID NO: 12, or the wild type of which BAG4 gene encodes a protein that has at least 90% sequence identity to SEQ ID NO:12, wherein the absence of functional HLS1 protein and/or the absence of functional BAG4 protein confers a microseed size to the plant grown from said seed.

19. A watermelon fruit produced by the watermelon plant of claim 6, wherein the watermelon fruit has seeds that have a pale seed color and optionally a microseed size.

20. A food product comprising the watermelon fruit of claim 19, or a part thereof, optionally in processed form.

21. A propagation material capable of developing into and/or being derived from the plant of claim 6, wherein the propagation material comprises the modified PPO gene and optionally a non-functional HLS1 gene, the wild type of which HLS1 gene is identified as SEQ ID NO: 7 encoding the protein of SEQ ID NO: 9, or the wild type of which HLS1 gene encodes a protein that has at least 90% sequence identity to SEQ ID NO: 9, and/or the seed further comprises a non-functional BAG4 gene, the wild type of which BAG4 gene is identified as SEQ ID NO: 10 encoding the protein of SEQ ID NO: 12, or the wild type of which BAG4 gene encodes a protein that has at least 90% sequence identity to SEQ ID NO:12, and wherein the propagation material is selected from the group consisting of a microspore, a pollen, an ovary, an ovule, an embryo, an embryo sac, an egg cell, a cutting, a root, a root tip, a hypocotyl, a cotyledon, a stem, a leave, a flower, an anther, a seed, a meristematic cell, a protoplast and a cell, or a tissue culture thereof.

22. A marker for identifying a modified PPO gene, wherein the marker sequence detects an insertion of a T between nucleotides 711 and 712 of SEQ ID NO:1.

23. A method for selecting a watermelon plant that produces seeds with a pale seed color, comprising identifying the presence of a modification in the PPO gene, optionally checking the color of the seeds the plant produces, and selecting a plant that homozygously comprises said modification as a plant that produces seeds with a pale seed color.

24. The method of claim 23, wherein the identification is performed with a marker for identifying a modified PPO gene, wherein the marker sequence detects an insertion of a T between nucleotides 711 and 712 of SEQ ID NO:1.

25. A marker for identifying a deletion on chromosome 2, wherein the marker sequence detects the presence or absence of a deletion corresponding to 13962 bp deleted between base pair position 4930 and 18893 of SEQ ID NO: 13.

26. A method for selecting a watermelon plant that produces seeds with a microseed size, comprising identifying the presence of the deletion on chromosome 2 with a marker for the identification of a deletion on chromosome 2, wherein the marker sequence detects the presence or absence of a deletion corresponding to 13962 bp being deleted between base pair position 4930 and 18893 of SEQ ID NO: 13. and selecting a plant that homozygously comprises said deletion as a plant that produces seeds with a microseed size.

27. A method for producing a watermelon plant that produces seeds that have a pale seed color, comprising modifying the PPO gene of SEQ ID NO:1, wherein the modification results in an absence of functional PPO protein, and the absence of functional PPO protein leads to the seeds of the produced plant having a pale seed color.

28. The method of claim 27, wherein the plant in which the PPO gene is modified has seeds with a microseed size.

29. A method for producing a watermelon plant that produces seeds that have a microseed size, comprising modifying the HLS1 gene of SEQ ID NO: 7 and/or the BAG4 gene of SEQ ID NO: 10, wherein the modification results in an absence of functional HLS1 protein and/or an absence of functional BAG4 protein in the plant, which leads to the seeds produced by said plant having a microseed size.

30. The method of claim 29, wherein the plant in which the HLS1 gene and/or the BAG4 gene is modified has seeds with a pale seed color.

31. A modified nucleic acid molecule, the wild type of which is identified as SEQ ID NO: 13, or the wild type of which that has at least 90% sequence identity to SEQ ID NO: 13, wherein the modified nucleic acid does not comprise SEQ ID NO: 7 and/or SEQ ID NO: 10, wherein the modified nucleic acid confers a microseed size to the watermelon plant when present homozygously.

32. The nucleic acid molecule of claim 31, comprising a deletion corresponding to 13962 bp being deleted between base pair position 4930 and 18893 of SEQ ID NO: 13.


SEQUENCE LISTING (20 sequences)

SEQ ID NO: 1 - DNA, 2744 bp, Citrullus lanatus
  PPO_WT_gDNA
atggcctctctatctccttccatgccactagcactttcctccgccgcaataaccacggccaccaccggcggcgcctcctttggtctgttttatcgtaaaaaaaaagatccatcttccaccattcatagactcaataacttggttgtgtgtagcggctccaatggcagtggtgaagaaagtaataactcattatggccaggcaagtttgttgaccggagagaagcgcttatcgggctcggcggtctgtatggctcagcttcaagtgcttttggagttgatcccttcgctttggcagctccagtcacaacccccgacccttccaagtgtggatcaagcacggacttggcagatggcgtcaaagatttggtttgctgcccaccatccaccaataacgtaaaacccttcctcaaaccacgcgttaggaaagcggcacaatcattagataaagaatatattgaaaagtataaggaagccgtagcgcttatgaaagcgcttcctgatgatgatccacgtagttttaaacagcaagcacttgttcactgtgcttattgtactgggggttacgatcaattgggtcttccagttgaattacaagttcatttctcgtggctgttcttcccattccatcgtttttatctttactttcatgagagaatattggggtctttgattaaggatcctgattttgcattgccgttttggaattatgacgctccgcaagggatggaaataccaaaaatctacacggataaaagttcgtcactctatgatgcatttcgtgacggacgtcatcagccgccgacattggttgatttggattacaatgacgttgagccaacaataagcagagaaaagataatccaatgcaatctaagtgttatgtatcgccaggtcgtgtccggcgcccgtacgcccttgctctttttcggccagccttatcgaagtggcagcaacccaagtccaggtgattttttttttcaaccatataatttcttttttcaaaaaaaggaaaagataaaaaagacaatcatatatagttatggttatgttaggtaatgtttttagtttttgaaaattaatgttaagagtgtttttgggttggattagagcagtttatagatctaactcaattgtcggattgataagttttttcaattcaaataaattctattaaataatgaatcgaattcaactcaatcatgaaatatatgggttcaaattacttaggtcatttatgtaaatttctgttctaaaaataagtaaaatgttaggaaactattataaatggaaaaaaatcaaagtttttacaaatacagaaagatttaattgagtctatcccctaatgataaaagtttatcgcggtttatcacggatagacaatgaaatatttgcaaataatttgactttttttgctatatttgaaaacaaccctaaaatgttaatatgtaaaatttttgttgaattatttttatatattgaattaaaattaacaactcaatttcaatttatattatgaagattttcttttcaaaatgctaaaatattattgtttgaagtttctggagaataaactttccaaaaaattatgaaattaaataaaattaaaataaatatatgtatatataattgtaccataagtaaatcttttataaaagaattataacaagttggggttatttgaagagaacacataaacaactcaaccaataactttttaatttatttgaactcaacctaacccaatcgaaacagtgatgattgggttgtgttgataagttatgctggtcatcaaattgacttgtttttttaatttaatcatttgaaaatgtattctaaacatgcccatttgtaaattagataatgatgtttagagataataattaaggatattagtatggaaagtaaaatgatgtaattttgatgaattgattaattttttgaaaggaatggggacggtggaaaaccttcctcacaattcaattcatttgtggacgggtgacccgaaccaatcgaaccgaattgacatgggaaccttcttctcagcggctagagatcccatcttctacgcccaccacgcgaacgtggaccgtttttggtccatatggaaatccttaggcgaaaagcgacaagacattaaagacaaagatttcctaaacgcttcctttgtattctacgatgagaatggtgaagctgtccgagtttatgtcaaagactgtctagataccagagccttaggctatgtctacgatgacaccgtaccaattccatggctcaaaacacctccaaccccacgagtaccacgcacacccaacaaaaccaagaagaaatctaccaagaagaccgggaagctaccgtcgagtgttgacaagatcgtcagcttcgaagtcaagaggccgaagaaatcgaggggtacgaaggagaaagacgatgaagaggagattttggtgattgatgggattgagttcgacggaaacaaggctattaagtttgatgtttttatcaatgacgaggatgatagggaaattagagcggataattctgagtttgcagggagctttgtgaatgtgcctcatatgaaaggtagcagcagcatgaacataaaaacatgccttaggttagggataactgaactgcttgagagtttggatgcggataacgacgatagcattattgttacattggtccctaggtttggagatgggtccgccaccgttaaggacattagaattgaatatgatgcataa

SEQ ID NO: 2 - DNA, 2745 bp, Citrullus lanatus
  PPO_MUT_gDNA_with INDEL
atggcctctctatctccttccatgccactagcactttcctccgccgcaataaccacggccaccaccggcggcgcctcctttggtctgttttatcgtaaaaaaaaagatccatcttccaccattcatagactcaataacttggttgtgtgtagcggctccaatggcagtggtgaagaaagtaataactcattatggccaggcaagtttgttgaccggagagaagcgcttatcgggctcggcggtctgtatggctcagcttcaagtgcttttggagttgatcccttcgctttggcagctccagtcacaacccccgacccttccaagtgtggatcaagcacggacttggcagatggcgtcaaagatttggtttgctgcccaccatccaccaataacgtaaaacccttcctcaaaccacgcgttaggaaagcggcacaatcattagataaagaatatattgaaaagtataaggaagccgtagcgcttatgaaagcgcttcctgatgatgatccacgtagttttaaacagcaagcacttgttcactgtgcttattgtactgggggttacgatcaattgggtcttccagttgaattacaagttcatttctcgtggctgttcttcccattccatcgtttttatctttactttcatgagagaatattggggtctttgattaaggatcctgattttgcattgccgttttggaattatgactgctccgcaagggatggaaataccaaaaatctacacggataaaagttcgtcactctatgatgcatttcgtgacggacgtcatcagccgccgacattggttgatttggattacaatgacgttgagccaacaataagcagagaaaagataatccaatgcaatctaagtgttatgtatcgccaggtcgtgtccggcgcccgtacgcccttgctctttttcggccagccttatcgaagtggcagcaacccaagtccaggtgattttttttttcaaccatataatttcttttttcaaaaaaaggaaaagataaaaaagacaatcatatatagttatggttatgttaggtaatgtttttagtttttgaaaattaatgttaagagtgtttttgggttggattagagcagtttatagatctaactcaattgtcggattgataagttttttcaattcaaataaattctattaaataatgaatcgaattcaactcaatcatgaaatatatgggttcaaattacttaggtcatttatgtaaatttctgttctaaaaataagtaaaatgttaggaaactattataaatggaaaaaaatcaaagtttttacaaatacagaaagatttaattgagtctatcccctaatgataaaagtttatcgcggtttatcacggatagacaatgaaatatttgcaaataatttgactttttttgctatatttgaaaacaaccctaaaatgttaatatgtaaaatttttgttgaattatttttatatattgaattaaaattaacaactcaatttcaatttatattatgaagattttcttttcaaaatgctaaaatattattgtttgaagtttctggagaataaactttccaaaaaattatgaaattaaataaaattaaaataaatatatgtatatataattgtaccataagtaaatcttttataaaagaattataacaagttggggttatttgaagagaacacataaacaactcaaccaataactttttaatttatttgaactcaacctaacccaatcgaaacagtgatgattgggttgtgttgataagttatgctggtcatcaaattgacttgtttttttaatttaatcatttgaaaatgtattctaaacatgcccatttgtaaattagataatgatgtttagagataataattaaggatattagtatggaaagtaaaatgatgtaattttgatgaattgattaattttttgaaaggaatggggacggtggaaaaccttcctcacaattcaattcatttgtggacgggtgacccgaaccaatcgaaccgaattgacatgggaaccttcttctcagcggctagagatcccatcttctacgcccaccacgcgaacgtggaccgtttttggtccatatggaaatccttaggcgaaaagcgacaagacattaaagacaaagatttcctaaacgcttcctttgtattctacgatgagaatggtgaagctgtccgagtttatgtcaaagactgtctagataccagagccttaggctatgtctacgatgacaccgtaccaattccatggctcaaaacacctccaaccccacgagtaccacgcacacccaacaaaaccaagaagaaatctaccaagaagaccgggaagctaccgtcgagtgttgacaagatcgtcagcttcgaagtcaagaggccgaagaaatcgaggggtacgaaggagaaagacgatgaagaggagattttggtgattgatgggattgagttcgacggaaacaaggctattaagtttgatgtttttatcaatgacgaggatgatagggaaattagagcggataattctgagtttgcagggagctttgtgaatgtgcctcatatgaaaggtagcagcagcatgaacataaaaacatgccttaggttagggataactgaactgcttgagagtttggatgcggataacgacgatagcattattgttacattggtccctaggtttggagatgggtccgccaccgttaaggacattagaattgaatatgatgcataa

SEQ ID NO: 3 - DNA, 1764 bp, Citrullus lanatus
  PPO_WT_CDS
atggcctctctatctccttccatgccactagcactttcctccgccgcaataaccacggccaccaccggcggcgcctcctttggtctgttttatcgtaaaaaaaaagatccatcttccaccattcatagactcaataacttggttgtgtgtagcggctccaatggcagtggtgaagaaagtaataactcattatggccaggcaagtttgttgaccggagagaagcgcttatcgggctcggcggtctgtatggctcagcttcaagtgcttttggagttgatcccttcgctttggcagctccagtcacaacccccgacccttccaagtgtggatcaagcacggacttggcagatggcgtcaaagatttggtttgctgcccaccatccaccaataacgtaaaacccttcctcaaaccacgcgttaggaaagcggcacaatcattagataaagaatatattgaaaagtataaggaagccgtagcgcttatgaaagcgcttcctgatgatgatccacgtagttttaaacagcaagcacttgttcactgtgcttattgtactgggggttacgatcaattgggtcttccagttgaattacaagttcatttctcgtggctgttcttcccattccatcgtttttatctttactttcatgagagaatattggggtctttgattaaggatcctgattttgcattgccgttttggaattatgacgctccgcaagggatggaaataccaaaaatctacacggataaaagttcgtcactctatgatgcatttcgtgacggacgtcatcagccgccgacattggttgatttggattacaatgacgttgagccaacaataagcagagaaaagataatccaatgcaatctaagtgttatgtatcgccaggtcgtgtccggcgcccgtacgcccttgctctttttcggccagccttatcgaagtggcagcaacccaagtccaggaatggggacggtggaaaaccttcctcacaattcaattcatttgtggacgggtgacccgaaccaatcgaaccgaattgacatgggaaccttcttctcagcggctagagatcccatcttctacgcccaccacgcgaacgtggaccgtttttggtccatatggaaatccttaggcgaaaagcgacaagacattaaagacaaagatttcctaaacgcttcctttgtattctacgatgagaatggtgaagctgtccgagtttatgtcaaagactgtctagataccagagccttaggctatgtctacgatgacaccgtaccaattccatggctcaaaacacctccaaccccacgagtaccacgcacacccaacaaaaccaagaagaaatctaccaagaagaccgggaagctaccgtcgagtgttgacaagatcgtcagcttcgaagtcaagaggccgaagaaatcgaggggtacgaaggagaaagacgatgaagaggagattttggtgattgatgggattgagttcgacggaaacaaggctattaagtttgatgtttttatcaatgacgaggatgatagggaaattagagcggataattctgagtttgcagggagctttgtgaatgtgcctcatatgaaaggtagcagcagcatgaacataaaaacatgccttaggttagggataactgaactgcttgagagtttggatgcggataacgacgatagcattattgttacattggtccctaggtttggagatgggtccgccaccgttaaggacattagaattgaatatgatgcataa

SEQ ID NO: 4 - DNA, 1765 bp, Citrullus lanatus
  PPO_MUT_CDS
atggcctctctatctccttccatgccactagcactttcctccgccgcaataaccacggccaccaccggcggcgcctcctttggtctgttttatcgtaaaaaaaaagatccatcttccaccattcatagactcaataacttggttgtgtgtagcggctccaatggcagtggtgaagaaagtaataactcattatggccaggcaagtttgttgaccggagagaagcgcttatcgggctcggcggtctgtatggctcagcttcaagtgcttttggagttgatcccttcgctttggcagctccagtcacaacccccgacccttccaagtgtggatcaagcacggacttggcagatggcgtcaaagatttggtttgctgcccaccatccaccaataacgtaaaacccttcctcaaaccacgcgttaggaaagcggcacaatcattagataaagaatatattgaaaagtataaggaagccgtagcgcttatgaaagcgcttcctgatgatgatccacgtagttttaaacagcaagcacttgttcactgtgcttattgtactgggggttacgatcaattgggtcttccagttgaattacaagttcatttctcgtggctgttcttcccattccatcgtttttatctttactttcatgagagaatattggggtctttgattaaggatcctgattttgcattgccgttttggaattatgactgctccgcaagggatggaaataccaaaaatctacacggataaaagttcgtcactctatgatgcatttcgtgacggacgtcatcagccgccgacattggttgatttggattacaatgacgttgagccaacaataagcagagaaaagataatccaatgcaatctaagtgttatgtatcgccaggtcgtgtccggcgcccgtacgcccttgctctttttcggccagccttatcgaagtggcagcaacccaagtccaggaatggggacggtggaaaaccttcctcacaattcaattcatttgtggacgggtgacccgaaccaatcgaaccgaattgacatgggaaccttcttctcagcggctagagatcccatcttctacgcccaccacgcgaacgtggaccgtttttggtccatatggaaatccttaggcgaaaagcgacaagacattaaagacaaagatttcctaaacgcttcctttgtattctacgatgagaatggtgaagctgtccgagtttatgtcaaagactgtctagataccagagccttaggctatgtctacgatgacaccgtaccaattccatggctcaaaacacctccaaccccacgagtaccacgcacacccaacaaaaccaagaagaaatctaccaagaagaccgggaagctaccgtcgagtgttgacaagatcgtcagcttcgaagtcaagaggccgaagaaatcgaggggtacgaaggagaaagacgatgaagaggagattttggtgattgatgggattgagttcgacggaaacaaggctattaagtttgatgtttttatcaatgacgaggatgatagggaaattagagcggataattctgagtttgcagggagctttgtgaatgtgcctcatatgaaaggtagcagcagcatgaacataaaaacatgccttaggttagggataactgaactgcttgagagtttggatgcggataacgacgatagcattattgttacattggtccctaggtttggagatgggtccgccaccgttaaggacattagaattgaatatgatgcataa

SEQ ID NO: 5 - protein, 587 aa, Citrullus lanatus
  PPO_WT_protein
MASLSPSMPLALSSAAITTATTGGASFGLFYRKKKDPSSTIHRLNNLVVCSGSNGSGEESNNSLWPGKFVDRREALIGLGGLYGSASSAFGVDPFALAAPVTTPDPSKCGSSTDLADGVKDLVCCPPSTNNVKPFLKPRVRKAAQSLDKEYIEKYKEAVALMKALPDDDPRSFKQQALVHCAYCTGGYDQLGLPVELQVHFSWLFFPFHRFYLYFHERILGSLIKDPDFALPFWNYDAPQGMEIPKIYTDKSSSLYDAFRDGRHQPPTLVDLDYNDVEPTISREKIIQCNLSVMYRQVVSGARTPLLFFGQPYRSGSNPSPGMGTVENLPHNSIHLWTGDPNQSNRIDMGTFFSAARDPIFYAHHANVDRFWSIWKSLGEKRQDIKDKDFLNASFVFYDENGEAVRVYVKDCLDTRALGYVYDDTVPIPWLKTPPTPRVPRTPNKTKKKSTKKTGKLPSSVDKIVSFEVKRPKKSRGTKEKDDEEEILVIDGIEFDGNKAIKFDVFINDEDDREIRADNSEFAGSFVNVPHMKGSSSMNIKTCLRLGITELLESLDADNDDSIIVTLVPRFGDGSATVKDIRIEYDA

SEQ ID NO: 6 - protein, 250 aa, Citrullus lanatus
  PO_MUT_protein
MASLSPSMPLALSSAAITTATTGGASFGLFYRKKKDPSSTIHRLNNLVVCSGSNGSGEESNNSLWPGKFVDRREALIGLGGLYGSASSAFGVDPFALAAPVTTPDPSKCGSSTDLADGVKDLVCCPPSTNNVKPFLKPRVRKAAQSLDKEYIEKYKEAVALMKALPDDDPRSFKQQALVHCAYCTGGYDQLGLPVELQVHFSWLFFPFHRFYLYFHERILGSLIKDPDFALPFWNYDCSARDGNTKNLHG

SEQ ID NO: 7 - DNA, 1982 bp, Citrullus lanatus
  HLS1_WT_gDNA
atggggtttaaaggctttgttattcgaagctacgaagagagtcaattatcagataaagctcaagttatggatcttgaacgaagatgtgaaattggccaatcaaaacgtgtgtttctcttcactgacactttgggtgaccccatttgtaggatacgtaacagtcccatgtataaaatgctggtaatctaatttaattttaattaattgtgtttttttataggttaattaattattaatttgtgaattgaaaattttttattattaaggttgctgagcgggacaaggaagtggttggtgttattcaaggctctataaaaccggttttttttactgctcataaaccgccgcccggtttggtggttaaactgggctacattcttggcctgagagtggcaccgccgtatcgccgccgtggaattggctctagcctcgtccgccgtttggaagattggttcctttctaatgatgttgattactgttgtatggccactgagaaagataatcatgcctctcttaatctcttcatcaataatttgaggtattttccatttttttctttttcttttaagtcaacaattatgaattgggagagagcacggatcgaacaatcatttttaaaatggtaattagtgtcattttatcttatgtgttatactcagattagctattaactctatcttataatgtaggttttgtcagtattttgagaactttcaaatttgtgtctaactagtttttttttccttagttacttaccaaacacgtgataatattattatatgatgaaattttttattttttttatttttttatttattttttattgtggcaagtaggagcatcatacttaccaacaacttaatagatataaaattaaaaatttaatggtcaaatcaaactttttcgagagtaattttaatgttgagttttgcacttttaaatataatttacgtatgattttgaaggattagattcatgtttagtttgaagtggtttagaatctggcaaaagggattttaacaatttcaaactaacgcctctcatatttgtagtttgggacgtcactttgctattaattaacaaaatcacatttttggaaattaacatatgattcacatgcaactcgtaaatgaagttaaacttgaaagtttagaggcatattagaaatttctttaaatattctttttcccaacaggtacataaagtttagaacaggaaggatcttggtaaacccagtaagaaatcatccatacaatatgaattcatcagaaatcaacattcaaaagctaaaaatagaagaagcagaagcaatatacaaaaaacacatggcttcaacagagttcttccccaaagacataaaaaacatattgaaaaacaagttgagtttagggacatgggtggcaaatttcaaacaaccgccatggtcgtcgtcgaactctgttggaggaaacgggcagactatggcgagtagctgggccattgtaagtctatggaacagtggggaagttttcaagctaaggctaggaaaagcaccatttccatggcttatctacacaaagagtttaaaaattatggataaaatttttccttgctttaaagtggttttggtgcctaattttttcaagccatttgggttctattttgtttatggattgcaccatgaaggccctttttctgagagattggttggagctttgtgcaaatttgtgcacaatatggcattgaataattcaaaggataattgtaaagctattgttactgagattggaggtgatgaggatgatgggctgaaaatggagattcctcattggaaattgctatcatgttatgaagatttttggtgcataaagtccttgaaaagtaagagatataataatattagtaatgataatgataacgataacgatcacgatcatcatatattggaatggacaaatgcctcacctaatagaactctctttgtagacccaagagaggtataa

SEQ ID NO: 8 - DNA, 1239 bp, Citrullus lanatus
  HLS1_WT_CDS
atggggtttaaaggctttgttattcgaagctacgaagagagtcaattatcagataaagctcaagttatggatcttgaacgaagatgtgaaattggccaatcaaaacgtgtgtttctcttcactgacactttgggtgaccccatttgtaggatacgtaacagtcccatgtataaaatgctggttgctgagcgggacaaggaagtggttggtgttattcaaggctctataaaaccggttttttttactgctcataaaccgccgcccggtttggtggttaaactgggctacattcttggcctgagagtggcaccgccgtatcgccgccgtggaattggctctagcctcgtccgccgtttggaagattggttcctttctaatgatgttgattactgttgtatggccactgagaaagataatcatgcctctcttaatctcttcatcaataatttgaggtacataaagtttagaacaggaaggatcttggtaaacccagtaagaaatcatccatacaatatgaattcatcagaaatcaacattcaaaagctaaaaatagaagaagcagaagcaatatacaaaaaacacatggcttcaacagagttcttccccaaagacataaaaaacatattgaaaaacaagttgagtttagggacatgggtggcaaatttcaaacaaccgccatggtcgtcgtcgaactctgttggaggaaacgggcagactatggcgagtagctgggccattgtaagtctatggaacagtggggaagttttcaagctaaggctaggaaaagcaccatttccatggcttatctacacaaagagtttaaaaattatggataaaatttttccttgctttaaagtggttttggtgcctaattttttcaagccatttgggttctattttgtttatggattgcaccatgaaggccctttttctgagagattggttggagctttgtgcaaatttgtgcacaatatggcattgaataattcaaaggataattgtaaagctattgttactgagattggaggtgatgaggatgatgggctgaaaatggagattcctcattggaaattgctatcatgttatgaagatttttggtgcataaagtccttgaaaagtaagagatataataatattagtaatgataatgataacgataacgatcacgatcatcatatattggaatggacaaatgcctcacctaatagaactctctttgtagacccaagagaggtataa

SEQ ID NO: 9 - protein, 412 aa, Citrullus lanatus
  HLS1_WT_protein
MGFKGFVIRSYEESQLSDKAQVMDLERRCEIGQSKRVFLFTDTLGDPICRIRNSPMYKMLVAERDKEVVGVIQGSIKPVFFTAHKPPPGLVVKLGYILGLRVAPPYRRRGIGSSLVRRLEDWFLSNDVDYCCMATEKDNHASLNLFINNLRYIKFRTGRILVNPVRNHPYNMNSSEINIQKLKIEEAEAIYKKHMASTEFFPKDIKNILKNKLSLGTWVANFKQPPWSSSNSVGGNGQTMASSWAIVSLWNSGEVFKLRLGKAPFPWLIYTKSLKIMDKIFPCFKVVLVPNFFKPFGFYFVYGLHHEGPFSERLVGALCKFVHNMALNNSKDNCKAIVTEIGGDEDDGLKMEIPHWKLLSCYEDFWCIKSLKSKRYNNISNDNDNDNDHDHHILEWTNASPNRTLFVDPREV

SEQ ID NO: 10 - DNA, 3637 bp, Citrullus lanatus
  BAG4_WT_gDNA
atgaaaaaatggtgttcaaaaggaagccaaattaggagcgaagagtatggaagaggagacgtagattgggagctccgaccaggtggaatgattgttcagaaacgacatgtcgggtcgggttcgggttcaaattcggagcgtttcattacaatcaacgtatctcatgggtcttatcgtcatcaaatcaccgtcgattctcattccacatttggtatgttatcatttcaatttgggggtttttttgaaatacagattgatttttattttaaattgaagactgaattattaatttttgtgtttgggacagggaatttaaagacagttttacgacagcagacagggttagagccgagggaacagagattgttgtttaaagggaaggagaaggagaacgacgagtggttgcatatggccggtgtgaacgacatgtcgaaactcatactcatggaagatcctgctactaaagagaggaagcttcaagagatgaagaagaagaataccactgctgcaggcgaagcactggcggggatcagagcggaggtcgataaactctccgaaaaggttcgttaaatcgttaaattacaactttagtcgaaaaatatatttgagaaaattgtaaaaaccactcgtggtaattacagttataccttcaaacttttaatattaaaaattaagtctttaaatttatattattgttaaaattggactcttaaattttgtttaattgtagaattgaagctcaaaatgataaaaattgaactctcaaacttatacaatttttaccatttctattattacttaagtttgaggtctcaattttaccataaaaaaatttaagaggtggaattgcaagttataactattatcatactttaaggtcaatttttaccatttgtctaggatattttttggttggtatggtttatgtttttaaattcttaatttctctaacattttgtgtttaataaatgggtaataatttatttcataaattttttaagttcacacctaaaattcaattttataactaaaaaattaattaaattttacttatttatttattatgatattcacatacttttaagatatttgaattctcaagtgaatttttttttaaacaacaagtttttctggaaattgacaaaaaaaagaaaaaagtagttttaaatgccttgcttttattttattttatttaatgaagttttgataatgatacaaatgtttatgtaacaaaaatgaaaacatttgaaagaaaaaaatggttattaggtaacatttctaaagtttaaaaacctatttgagacaaatgtgaaagttcaataactcattgaattgctttagaaggtttaaaaaacaaatagttacaaacaagcttagagactaaacttctaattgaacctaattctaaatgattgaaatgaattgaccaatggataactagcatattatacatttttttaaaaaaaaaaattggaaaggggcaaaaaagtgtgagtgtataaaattagggtttttaagtgcaggttgcggcagtggaaggtagtgttaatgacgggaagagggtggaagagaaagaagttaatttattgatagagttgttgatgatgcaattgttgaaattagatgcaattgagacggatggggattccaaacttcaaagacgaactcaggtatctattggactatatgtcaattatcattaaaaataaatttactttggcttatctatttataataattagggtatacaaatttaaggtgatttaaatcgttttctttcattaatctaactatgcaaaaccgttacaacaatacgtcacctttaaataactttaactatttaccaaaactttatgaagaggaattataaatttacttaccgcctaatttctcttttaaaactctttttgttaactcttaatgtcgggtatgtttgcattagtcatatttaatatccattaaatgatataacttttcaaacaataataattaacatatatctttattattattattagttattagatttgtatagttttctaaaaaaaagaatggattttatgtaagtttggattaacttaaaaaataaatatttttttaaaaattatttttatttaaatttttgtgacaaaaactttttaaaataaaaacactaaattccatttggatgaattatatatttaaatattatatttctatggtagaaaattatttactattatttaatcaattttaataatgatggattaaatttaagttttattaaatgaatacttgaaaatatgaattaaaattaaatatatatattttaatttttcaattttggtatttataataaaaagtacgatagtttaatcattaatgggttaggtggctggtgctcccctagggccatcaaacttaaaataattaaaaataatgaaagtctcctaaattgtatgaaaattcaatgaatataaattgtgaaaaatgataatgggtattttatctatttatttattaactcaaaaaaaaaaattaataaatatagactaaaaaaattgcagaaataggacaaaatgattttaattctttcccttgatatgacatttttatgtgggacattatgaaaccaagaacttatcaagaaggattctattcaaaataaataaataattgattaaagaagaaaattccattaatgtccctaaagtcttaatcacacctctatttagcgtctatcatgaataaaataaatagaaatcataggaatgctgaggtggcatgaacactagataaaaattttaggtttaaatactactttagtttttatatttttcacattgtttcatttagattcacgaccttttaattttggttaaattataaatttagtccatataatttgaagaaagttaaaatttaatcctatagtttataattagaatttaatctctatgatctgataaaatcctcataaataatctcactactgtagagactaaactatagggaacatttataaggttttatcaaaccataggaactaattctagattttaaaaccaaatggaccagattttaattttctccaaactacaggggccaaattctaattttttctaaattataggagacaaatttgcaatttaacctttaactatagttaattttggtccacttactttcaaaatatcaattttagtcccgtggttttaaaaagtctccattttggccccttaacaatgaacaaaaataagataaaaatagtaattaaattttaattttgaactatgtaattttttttttgaagtacaaatagtagagtagggaaattgagagaaagagtatacgttaattatcattgaactatgtttattttggtggtgataagtttttacgcaatttcaattaatttgaataacgttagaattgtaattttataattttgggaataaaacaggttgttagggtacagaaattagtggacagaattgacaagttgaaggttagaatctcaaatcctttaaaccaaacaacaatgaaaagaggcaaatgggaggaatttgaatctggatttggcagccttattcctccaacttcaaaactcaccatcagctctacaaaaataactcatgattgggaactctttgattag

SEQ ID NO: 11 - DNA, 807 bp, Citrullus lanatus
  BAG4_WT_CDS
atgaaaaaatggtgttcaaaaggaagccaaattaggagcgaagagtatggaagaggagacgtagattgggagctccgaccaggtggaatgattgttcagaaacgacatgtcgggtcgggttcgggttcaaattcggagcgtttcattacaatcaacgtatctcatgggtcttatcgtcatcaaatcaccgtcgattctcattccacatttgggaatttaaagacagttttacgacagcagacagggttagagccgagggaacagagattgttgtttaaagggaaggagaaggagaacgacgagtggttgcatatggccggtgtgaacgacatgtcgaaactcatactcatggaagatcctgctactaaagagaggaagcttcaagagatgaagaagaagaataccactgctgcaggcgaagcactggcggggatcagagcggaggtcgataaactctccgaaaaggttgcggcagtggaaggtagtgttaatgacgggaagagggtggaagagaaagaagttaatttattgatagagttgttgatgatgcaattgttgaaattagatgcaattgagacggatggggattccaaacttcaaagacgaactcaggttgttagggtacagaaattagtggacagaattgacaagttgaaggttagaatctcaaatcctttaaaccaaacaacaatgaaaagaggcaaatgggaggaatttgaatctggatttggcagccttattcctccaacttcaaaactcaccatcagctctacaaaaataactcatgattgggaactctttgattag

SEQ ID NO: 12 - protein, 268 aa, Citrullus lanatus
  BAG4_protein
MKKWCSKGSQIRSEEYGRGDVDWELRPGGMIVQKRHVGSGSGSNSERFITINVSHGSYRHQITVDSHSTFGNLKTVLRQQTGLEPREQRLLFKGKEKENDEWLHMAGVNDMSKLILMEDPATKERKLQEMKKKNTTAAGEALAGIRAEVDKLSEKVAAVEGSVNDGKRVEEKEVNLLIELLMMQLLKLDAIETDGDSKLQRRTQVVRVQKLVDRIDKLKVRISNPLNQTTMKRGKWEEFESGFGSLIPPTSKLTISSTKITHDWELFD

SEQ ID NO: 13 - DNA, 23333 bp, Citrullus lanatus
  cl_97103_v1_Chr229897185-29920517
ttgttacctaataagaaatttagactttagtatttagtttttgaaaattaagtttataatattactttcacttctaaattatatgttctgttgtcactcttttatcaatatttttaaagtttcatttaatgaccatttgatttttagtttttgaaaattaagtttataaacataacttttacctattggtttctttgttttgaagtaagttttgaaaactaaaaacttaaaaaaagtcatttctaataatttttttttctggaaattggttaagaatttaagtgttcagtaaaggaagacgaaaaccaagtaaccatgataagaaagaatgtaagaaaataaacataatttttaaaaactcaaaactaaatagttatcaaaccaaaaactagtaattcaaataaatatatatataagtacttgaactagccccaaccaaagtacttatttgattgagtaaactacaattaaattaaaaggttaaaatatttaattaatccttaattaactaaaaatatcaatattgacaactcattatggaccatattactctctctctctctcttaataaaagaaacttaatataagttgaaggttggtccattggtttattttattttaaatctttttagcccttcttaaatttttaattttataaaaacaaatttattagtcaccaaaaaatggttgcttgtaggatgaatgaagacgaaatactaaataattttattttattaccacgcgtggccacagaaatgttatttttaaaattttaatataaagtttgtgaggccaattattttttaattatgctaaaaacattcagctttgcgtgagttggaacttggaatacacaacatattatttgaccattgaaaatccaaaatctgaatcacaaccctctaattttattctgttattattatttatttggattgaagtaatccagccattcctttacttatttatttatttaatataaggtaagtgttacaaaatcgataaacaaagaatacaaaaaatataaaaagatagagaatcgacatatagatttacatgatttactaacagtgtgttagttacgttcacagaacagatgaaacacaattttattagagataatgttgcagaatacaatacagtgacacctctatctttagagaatttatatagtgcactcatttaaaccttagggatcataatcgtaattaaccataaataattaaatatatcaaatacgaatgcccccaaatccccaccagtaagatcccatacttagtcatttgaagtgccagatagcatacaacattattcaaacaatatgttagttgaccaccttgactttattgaaaaaggattcaaatcgaacctagtaagattgcatttacaatagattaagaaagttcatgaaagaataacacaattattttctttccaaaaaacacacacaataattttacgtggaaaccctctaaacaatttaaggcaaaaactatggataaagataaaagaatttcactatataaaataggtattacaatttgtttacagactctctcgtaagataaaatctctctattttatatctcaatcttttctcacttaattctggtttgttaagcaaccatgggttctgacctcgttataactaaaagaaatttagccatcaaacaaattcaatggtcccaaaatgactttgaataatgtgaaagttttaagggtcacaaattgaccttgaatggagtgtaaaatctaattaaccacaaattggcctacaagaaaatgtattgaataatcttgaagataatattatttaactaacttctcataagaaaaattgttattttaattgttgagtcgctcacgttgaaagtttaaatcatttcaattattatgagaatgctattttttctccttttattttaatgaataatttgagcacaactcagttagttaaggtatatattctcgaccaagaaatcagacattcaaatcctctccccttacatataggataagaaaaacaataaagtttagaggcatattagaagttcaaatacttattagaaacaaattaaaattttattgttcaattagacacaccttaaatttataactaatagatcaatcatttaaaagtttcgaacgtgtcaaaaatttattgtggcacaaattttttaaaacaattaaaatactaaagattaaatactattttgactcactactttgaagttaaatttattttggttttcatacttagaaaaatgttgactttgatttctttagttttcatgtttggtttattctactcttacactttttaaaccttcgttttaatttcaatttttttttcttttaaaaatgtgaaatgttttgtcctttttttattattatattattttaagaataataataaactcatgatttagaatttaatttcagcgtaacactacgagcgatttgatttgaaatgtaattataaccgaggggtaaatttcgcggcccatttaacagaccatttacaaaacttgagccgggctgccaccatgtgggctgggtgtcaaatgcaactggtgaagtggcctgctgatgggccgctccatccaacccattcaaaccttaaaaaagagttaaaaaatattactacggttagttttggacgaaatcctatgaactttcaaaattataaaaaatattcttcaacttaaaaaaaaaaaaaaaaaaactatctttcctattaatatataaatagaaactatttatacgtcgttgcaaaaataaatatgatatttgagatttttttttttaaatgctcgtatagtatataattttttttttcttcatatagactattcttacatctattttctcagaaaccaaattaaaaacataactttaagttaaaacataaaatctcacaaaattcaaaaatcaaaatctctcaaaacacaccaaacacttaactacttatgtacagttttattaaatcattcaagtcgtaaaaccctttgatgcttccattattctttttcttcttattagaatctatgtaactttaattgaagtgagcatatttagagggtattgaagaaaagaaagaagaaaaaacagggaatagtaaaagtatttttaaactatttttcaaggtttaagaaaatttttgaaagttttggaagtttaaaggtattttagcacaaattacatgttttatttttaaattgtagatttcataaacttccgtcgttttctctccttcattttcgtaaacaaaaatcgttttcaaaattcaattgttatccacataaaatttatcaaataattttttgtttgtttgttcaaaccattatttaagctgcaaattttaatttttacagttttggttgacacaaaacaaaacttgaaattacctcaacatgcatataaacactagttttgagccacaaatcactatggatctatagtggagtggaataaaggtatatgacataaatttttagcataaaaattatctactattatttttggttggtctccttttcttatttgatcatgcattttcaacataagcatttttcatacttgttttgctccgatgcatttttatattattcgatgggaggtgggagaaaaataataagtatcttgacttcatatgactcaagtaaaacttcggattcaaccctacaacatttcactagttgtatttaggggctagcttacattttgaaataagtaggtaataattaattgcttgactatgtatagtgattgtaataaggcttagcaataataaaagatggtttatgatttctatttttcctttctaaagtatggccgataccattttcataaacgttaaccattacaagagattgatataataacattgctaacgttgttaacaaatgtattatctatacaaaataatgttttcaacaattacaatagggaagaaaacatggaaaagtagtgcttcacactttgggcaaatcttgtttaattaattaatcttgtttcgaatataataatgcaatattttctttttatcagacttaaaatttatcgattatttgggctgatattttgcataattatttttataattttaaaattagatacttgatcaagttgttagaatctttcttgtttactagattttctccaacttctatgatttgtttggttttcaatattcatggcttttagatttgatacgagggtatttctgtccatcaattttctagttgctatataatttacttttcaaatatttgaactaaatactgatctatttgatatattaattattttgtttctatcatgatgtttttttttttttttttttttataatttttaatggcttggtattctccaagtgcaattaaccatttaaatgaaaattttggtaaaaatttactatttgattaaaataatttgaaaatcaagtttattttgagatataacctcatgcttatctaacatggtatcatagttataaagttgatattcaatctcaataaaaaaaaaaaaaaaaaaaaaagaaacattgagatccaattcaataagagtgaaccaaaagatccaccatctcaaagggcatgttgagtggtcatactttgaaaaaattgagagacttcacgtgaagatacttaaattaattaattaatattttaatgataacaaatcttattttatatttaatgttttgaagagtttattgttttgagttcagaataatgattccaacgtaataattactcaaatagtagttcaaaagaagaaaatataacaaacattctaagttaaccaattgaaatatgacatttgaagtatatgggaaaaatgacacatgatggttttttgacttttaaattgatttaaaaaaaataaataaatttaatattatataggtttatgttctatccactagttgacttaaaaattaataataataattaaaaaaaaaaaaaagtgcatgtagctgttggagatcactttaaagtagaagaattttttttttttaatttttaatctaaaccatttaatattctctttaggatgatttaaatactcaatgaatttggatttcactattctcttttgaaactcttcatctttttcacatttcaacaacaattttcataatttttaattctctaaaaaaaatacaaagcttccattgttactttcttcaagcttcattttttttttcatcttatttttaagagagatttatgtattgtgagaattgatttgttttataagctcaaatttgtgaatcctacttctttaaagagtttttctcatcttgttcttcattttcaaattcatattgtaagtggttactcttgaacctgtgagaagagtgtggtaacaatttgatcatcaatggtggaaaacatcgagtttattcttgtgcctggaaaaagaacgttgtagcagttcacctcaagctgtggaatgaatcgagtttgagtgaccatatccaagagaaacttagggagtggatgtaggtcgggtagtgtcaaactactataaaatgtgtcaatttcctctctacgtcttctaatttatttttgcaattaatttgttattatattttattccatgaaattaattgcaatatttattccttaaattaagtgcttacacattgatttatttagatgggttgaattgatctttacatatcctttaccaatcttcttgaatcaaatagggttctataaaatattagtgttaatttattatctaattcaagatgaatttatttgcatgaaattattgtttaagttacttgctcgcaaaaagtatattgatttgtttaattgggcccactgtagtaaaaagttttaattaatttaataaactctattcacccctttaaggttgccataccagtcctacaatgagatggaactcatgtacgtttatctaataccaccctaatgcctgcaaaatttataacgaggggtgagaaatgagaaagaggcgatctcccatttccgtccccgccccatctcgtaaacatccctaatatataatccaaatgcatttttctcacaagaatgaattctctttcaagtggtccaagagggaagaagtttgttttcaaataacgtaatcaacgcaagctgaaataaaacagcagccatttgagaatcaaacacaaaaaatggaaaatttgtatgtgtataattaattaaccatcaaaatttcataaacaaataaacacttcaaatcaacttcacattgtccataaattgcaaagacatatttccattttaggaagaataaagttttggcctaactcatccatggctgtgttttttaaaagccctaataataatatattccaaaactctcctctttatcttttttctcaaatctctcccctttttgccctttgcccttcccccacctggccttcctttaaaccctaataattccaaactctcaccttttttaagctcaacactcaatatttaacctaatctctatttatatgtatatctttcttctttttcactcttattattgcctttgcatatagcacatgtgatgagaatattccccaaatgcctctcctcttattgtttttcaactttgaatctttataaagtaaaaagatgggtgacacctaaattaggaatgtctaaaggtttttttctcttaaatgacacacaaaaaaacataataataagatgcaaaatctagacatttcaatccacaaaagaattaacattatcgtttgattattaattatttggactccaaagcaatagtacatatactattaatagaaaacgacgttgatattttttgcattcatatttagtcatgaatacaataatacgaatagagacctagttaaatccagaaacatccacgtacaaaaaaaaacaaaattttacaacataactatttcagttcagatcgaaatcgtcttcttctttctttataacttttcttctactttcttcactctgctatcaagtacaaacaataattagattgagacatatcgagagagggttaaacctattttctttttattcttttatacctctcttgggtctacaaagagagttctattaggtgaggcatttgtccattccaatatatgatgatcgtgatcgttatcgttatcattatcattactaatattattatatctcttacttttcaaggactttatgcaccaaaaatcttcataacatgatagcaatttccaatgaggaatctccattttcagcccatcatcctcatcacctccaatctcagtaacaatagctttacaattatcctttgaattattcaatgccatattgtgcacaaatttgcacaaagctccaaccaatctctcagaaaaagggccttcatggtgcaatccataaacaaaatagaacccaaatggcttgaaaaaattaggcaccaaaaccactttaaagcaaggaaaaattttatccataatttttaaactctttgtgtagataagccatggaaatggtgcttttcctagccttagcttgaaaacttccccactgttccatagacttacaatggcccagctactcgccatagtctgcccgtttcctccaacagagttcgacgacgaccatggcggttgtttgaaatttgccacccatgtccctaaactcaacttgtttttcaatatgttttttatgtctttggggaagaactctgttgaagccatgtgttttttgtatattgcttctgcttcttctatttttagcttttgaatgttgatttctgatgaattcatattgtatggatgatttcttactgggtttaccaagatccttcctgttctaaactttatgtacctgttgggaaaaagaatatttaaagaaatttctaatatgcctctaaactttcaagtttaacttcatttacgagttgcatgtgaatcatatgttaatttccaaaaatgtgattttgttaattaatagcaaagtgacgtcccaaactacaaatatgagaggcgttagtttgaaattgttaaaatcccttttgccagattctaaaccacttcaaactaaacatgaatctaatccttcaaaatcatacgtaaattatatttaaaagtgcaaaactcaacattaaaattactctcgaaaaagtttgatttgaccattaaatttttaattttatatctattaagttgttggtaagtatgatgctcctacttgccacaataaaaaataaataaaaaaataaaaaaaataaaaaatttcatcatataataatattatcacgtgtttggtaagtaactaaggaaaaaaaaactagttagacacaaatttgaaagttctcaaaatactgacaaaacctacattataagatagagttaatagctaatctgagtataacacataagataaaatgacactaattaccattttaaaaatgattgttcgatccgtgctctctcccaattcataattgttgacttaaaagaaaaagaaaaaaatggaaaatacctcaaattattgatgaagagattaagagaggcatgattatctttctcagtggccatacaacagtaatcaacatcattagaaaggaaccaatcttccaaacggcggacgaggctagagccaattccacggcggcgatacggcggtgccactctcaggccaagaatgtagcccagtttaaccaccaaaccgggcggcggtttatgagcagtaaaaaaaaccggttttatagagccttgaataacaccaaccacttccttgtcccgctcagcaaccttaataataaaaaattttcaattcacaaattaataattaattaacctataaaaaaacacaattaattaaaattaaattagattaccagcattttatacatgggactgttacgtatcctacaaatggggtcacccaaagtgtcagtgaagagaaacacacgttttgattggccaatttcacatcttcgttcaagatccataacttgagctttatctgataattgactctcttcgtagcttcgaataacaaagcctttaaaccccattaattagaaaaaacaaaattaaaagataaagattgagagtgagtgagtgataaaaatgagaggagaaatttatttatataataaggggagagagaagaagggtttgttgtaaatatggtatttgatatcaacatcaagatggtaggataaattttgaaaaggtgtttaatacgagacaaaaagtgattaatgatagaacgtgttccttgtctatggtttaccctttcttaacttttcttttacttattataggcagaccatgttaggataactatatgccctcacaaaattctctctctctctctctctcttgtttattcaattaacatctcttttctattttaatttaataaaatacaattaacttatgattcactcaattttctcactcaacacattgaaaatgaccatcttaatccctgtttgcaaaacgttgatataaaattttgaaaaaaaaaaagactaaaaatgaagattgatggaccaaaattgtcaaccaaaatatattttgaaagttcaagaatcaaattgaacattttggattaaaatagattaaattgcaaatttggtcgttatggttaggaggactaaattctaatttttttcaaaaaatcatagggatcaattttataatttgaccattgaaataccattttaatctacgtattttgagtataattccattttaatacatatactttcatttgcgtaaatttaatcttaaattttcttttatgaaaatatattaaaaataataatatattatcaaaatttagaacataaggctaatacaaagcagaataagtaaatggaagcgtatatgaaaataatatttaaataaaataaaataaaataataatagagatgaaaatgaaagctaagaaagaatggaattggaaaagattgtattacaaagacataatgagtacttttttccgttaccaccttttgaaaaatgaggaaaactagaaattcttaccttaaaaaacgccatatatacaaagggcaaaaacagatcgatgacaagaaaattccaatttcaaggccaatcaatttttcgctttctttttattaaaaacaaataattattattattattattattatttttcctttgtcaagcattgattgattcacattttaatggaaaggatgggatttcgttgaaaatgggtttaaattaatggttaatttggggtggtccactccattgcttccataatttcttagcttttaaaagacatttatgagatataaatttagatattgcctaagaaagtgaaagtgaaatggccgtaaatgaagcccatatttattttgtagttactagattaagtcctatttggtaatcatttcgtttttgttttaaattcatctctaagtttcaagatttcactaaatactcacttccaaaataattttggagttaatatttttttagttaatttaaaataattatgaagtaaaattttaaaattaatttaaatagtaatggacaaatagtgaaacttattcaattttaattcttttaaataaattaatagataattaacaataaaggctaaaagtgagtattttgtgataaatctaagttaaaaatataagtcttcaaacataggaaccaaattgaaacaaagtttaaatcccaatggtaaacttgtaatattttgaaatttagggactaaactgaaatcaaactctttaaaatctatggaccaaatgaaaactaaacgtaaaacttaaggtccaaaaaagagtatacgaatgtgttagtgaccaaaaactagtctgtattttgaatttttctatctttattgtattgaaaaaaaaaaaattccttctattgaggaactatttatttttaatttgttaagtggaaaaatgatatgatcgtaagggggcccctctttcacaccatgtctttggcaaacgtgtggtagtgaatgataagaaagccaataggtgcaataatatgcctatagaaacttttatgtcaattgcatttttttcccccttagtacaatgggaatttaatttgatgacatgatttttcaagtctcaatcaagttaggattcttgatttgaatattatcctaattaaggtattatttaaggagctcggtgcaatatagttaacgatatcatattgatggagacaaaattattgctttatcatacgtttaataatttagaagataacgtttacaatataacatcacagtccatataaataattgttattacgtactttttcttttcaaatattacaatagttatggagagaatacaaactgaaccatttttttagtctaacaattgcaaggaatgggaactaaattgttgacctttgatgtaatcaattaaagaattaaaagagagaaaaccaaaaagaggaaggagtgtacaatgtgtcgagtaatagtttcgattgaatgaatgattagtgaggggttgtaataaggtatttttaggcataaggggggtgaaaaatggcagcctggcactttttttctaatatttccaatgatttctgtggaccactccccttcccctctccctacatgtttttagaaaaaattaccaataaggaatccaaccctaattatttcttccatttcttattataaaccttttctctcccttcatatttcattatcattattatttgtttttctaagtggggataattttttattttttatttttttaatatatatatacttttttttttttttatgttcattggccctctctttttaaaatgtttctaaaattatgatagttttagaccaagttaaatgtcctcaaaacacaatttcatgaatatagcgattttgtttgatgaatgaaatttgaaaatattttattttttttctaaaaatgaaattgttccattaataatttgctcttcaatgtatagatgaaaaagtctaacttcattgattgtcaaatcatccttttttattttcaatgttatattaatgtttaaaatttttcgataaatgtcaagttgatcaagtgatatatatctaatctcatttgagccattagaaatcttcttataaaccaatagtgtcaatcaagctattaaaagagatgttattgatgatgatgtttgacaatttaattttttatgtctagaaatcttctttagaagctgggattttctctatattattccaatcttctttataattttttaaaaatattaattacatttcatttttctaaaaatatagtaaatataaaaaataaaaaaaaatccctttgaagtttttcactctatttacaattacattcttttaaaagtcgtaatattacttttgtacttttataagttcattttccttctctctcttattattttgttagtatttatcgttatatttttttagcacaataattgtgggattcgaacttttgactttttttaagaaaaaaaatctacgtcttatgtcacttgaactatgctcatgttggcttataatgatggtataattttttcatagttagaattctatcgctaacatacttttaattcataacctaaatctatcaataatagagtattatgatggatagatttcaatcacattgcacaactaacatatatatatatatatatatatatataatttatctcttctatcaaagatagatttcaaacaaataattttcctttttgtttccctccattttcctccaattttattctctcatccctttcattaataacccaacaaaatattcaaagtaatttgttacaatatccttttcaaagtaactagaagaactagaaacaactaaattgcacttaaactgtctatgactgaagcttaaaagtacataataaaaatctataagtagtcttatatgtaagtgatagattttaatttgctattggtgatacctcatatcattgatagggtctactcatgatagagagtaaatgataaactctatcattgataccaatctatcaatgatatacttctattgttgatatatttcaagtaatataagcctacaagtcatagacttctattaccgatagactctatcactaataaacttcgatagttaaatctaagttttggtatatctataaattctattatattgatatattatatatattaatactttgaacattgttgtatttacaacaataatatacttcaaattactaagatagcagccgaacatcagaacccatgggcttgggcccaataatatcatggcacgaagtacaaccccatggacatcgcatgggttattaagcccaccaagagcctaaatcacatcaagttcaagccatgatcagaggcctcaagaagcccacgggtatatgtgggccagcaaagcccaaaaaacttgggccaaaggcccaattaaaggaattgtcgtacgatgtggacagttggacatgtacaattccctttttaatcacgaactttaaagttgtgaccgaccaaccgctgttcgtaatgactgtttgaaggtccctttcatcaatccactgacacttgaaaagtcattaaaatctccgtcgctattatgtagctgtctcatactaatttaacctaacttttaaactttaggctaagtttacatgttattaactttcaaattttacatctattaggtgtcctcaacttttaattaagtgtcttaatgagattttagtttttaatacattcttaaattttcaattttacgttgaatatatatttaagaccttataaacacttattctaactcaaaatttattgatttgttatctactttttttaatatatttttttaaaatcaatctaaattttgaaatctaaaaagatttattattatttatttttttatttttaaaaaagcttgtttttggaatttggaaattgagggatgcttaatttcaaaaaaaaaaaaaaaaaatgaaatggttatcgaacaaaacttgaccttttaaattcatgtatctattaaacataatgtgaacttttaccgtttctttagatataagatccaattttatgtcaaataggttaattacaaatgaattaagggtagtagttttaaatagcaaaactactagaaatatttacaagtatagaaaaatgtctctgtttattagtaatagacaatattgatagacatgtaccagtgtctattactaatagacaattatagatgttagtaatctatcagtgataaatatgatattttgctatattttaaaatttttttatatttgaaaataactcatgaattaatttatcaaatacttattatatacaaaattataagtttaaaaatttattagacacaaaatttaaagtttaacttattagacacatctatctattagtgtccggttgggtttggattcaacagtttcgttatatctaaaaatatttataattaaaaaaattaaaatacacgccgttattaaattaatttgaaagtttagagactaaataaaagggaaaaaaaaaggaaagttggaacgattaattgtcaaccacgtaaaaaggacctgataggaataattcttcaatgacacactctccccctcttcttttgttataatttcgctttcatttcactcacactctcacatcatccaaccaaccaacacaacacacaccaccattttttttcaatttggagccaaaaaaagaaaaacggaaattgtttggtaagagagatgaaaaaatggtgttcaaaaggaagccaaattaggagcgaagagtatggaagaggagacgtagattgggagctccgaccaggtggaatgattgttcagaaacgacatgtcgggtcgggttcgggttcaaattcggagcgtttcattacaatcaacgtatctcatgggtcttatcgtcatcaaatcaccgtcgattctcattccacatttggtatgttatcatttcaatttgggggtttttttgaaatacagattgatttttattttaaattgaagactgaattattaatttttgtgtttgggacagggaatttaaagacagttttacgacagcagacagggttagagccgagggaacagagattgttgtttaaagggaaggagaaggagaacgacgagtggttgcatatggccggtgtgaacgacatgtcgaaactcatactcatggaagatcctgctactaaagagaggaagcttcaagagatgaagaagaagaataccactgctgcaggcgaagcactggcggggatcagagcggaggtcgataaactctccgaaaaggttcgttaaatcgttaaattacaactttagtcgaaaaatatatttgagaaaattgtaaaaaccactcgtggtaattacagttataccttcaaacttttaatattaaaaattaagtctttaaatttatattattgttaaaattggactcttaaattttgtttaattgtagaattgaagctcaaaatgataaaaattgaactctcaaacttatacaatttttaccatttctattattacttaagtttgaggtctcaattttaccataaaaaaatttaagaggtggaattgcaagttataactattatcatactttaaggtcaatttttaccatttgtctaggatattttttggttggtatggtttatgtttttaaattcttaatttctctaacattttgtgtttaataaatgggtaataatttatttcataaattttttaagttcacacctaaaattcaattttataactaaaaaattaattaaattttacttatttatttattatgatattcacatacttttaagatatttgaattctcaagtgaatttttttttaaacaacaagtttttctggaaattgacaaaaaaaagaaaaaagtagttttaaatgccttgcttttattttattttatttaatgaagttttgataatgatacaaatgtttatgtaacaaaaatgaaaacatttgaaagaaaaaaatggttattaggtaacatttctaaagtttaaaaacctatttgagacaaatgtgaaagttcaataactcattgaattgctttagaaggtttaaaaaacaaatagttacaaacaagcttagagactaaacttctaattgaacctaattctaaatgattgaaatgaattgaccaatggataactagcatattatacatttttttaaaaaaaaaaattggaaaggggcaaaaaagtgtgagtgtataaaattagggtttttaagtgcaggttgcggcagtggaaggtagtgttaatgacgggaagagggtggaagagaaagaagttaatttattgatagagttgttgatgatgcaattgttgaaattagatgcaattgagacggatggggattccaaacttcaaagacgaactcaggtatctattggactatatgtcaattatcattaaaaataaatttactttggcttatctatttataataattagggtatacaaatttaaggtgatttaaatcgttttctttcattaatctaactatgcaaaaccgttacaacaatacgtcacctttaaataactttaactatttaccaaaactttatgaagaggaattataaatttacttaccgcctaatttctcttttaaaactctttttgttaactcttaatgtcgggtatgtttgcattagtcatatttaatatccattaaatgatataacttttcaaacaataataattaacatatatctttattattattattagttattagatttgtatagttttctaaaaaaaagaatggattttatgtaagtttggattaacttaaaaaataaatatttttttaaaaattatttttatttaaatttttgtgacaaaaactttttaaaataaaaacactaaattccatttggatgaattatatatttaaatattatatttctatggtagaaaattatttactattatttaatcaattttaataatgatggattaaatttaagttttattaaatgaatacttgaaaatatgaattaaaattaaatatatatattttaatttttcaattttggtatttataataaaaagtacgatagtttaatcattaatgggttaggtggctggtgctcccctagggccatcaaacttaaaataattaaaaataatgaaagtctcctaaattgtatgaaaattcaatgaatataaattgtgaaaaatgataatgggtattttatctatttatttattaactcaaaaaaaaaaattaataaatatagactaaaaaaattgcagaaataggacaaaatgattttaattctttcccttgatatgacatttttatgtgggacattatgaaaccaagaacttatcaagaaggattctattcaaaataaataaataattgattaaagaagaaaattccattaatgtccctaaagtcttaatcacacctctatttagcgtctatcatgaataaaataaatagaaatcataggaatgctgaggtggcatgaacactagataaaaattttaggtttaaatactactttagtttttatatttttcacattgtttcatttagattcacgaccttttaattttggttaaattataaatttagtccatataatttgaagaaagttaaaatttaatcctatagtttataattagaatttaatctctatgatctgataaaatcctcataaataatctcactactgtagagactaaactatagggaacatttataaggttttatcaaaccataggaactaattctagattttaaaaccaaatggaccagattttaattttctccaaactacaggggccaaattctaattttttctaaattataggagacaaatttgcaatttaacctttaactatagttaattttggtccacttactttcaaaatatcaattttagtcccgtggttttaaaaagtctccattttggccccttaacaatgaacaaaaataagataaaaatagtaattaaattttaattttgaactatgtaattttttttttgaagtacaaatagtagagtagggaaattgagagaaagagtatacgttaattatcattgaactatgtttattttggtggtgataagtttttacgcaatttcaattaatttgaataacgttagaattgtaattttataattttgggaataaaacaggttgttagggtacagaaattagtggacagaattgacaagttgaaggttagaatctcaaatcctttaaaccaaacaacaatgaaaagaggcaaatgggaggaatttgaatctggatttggcagccttattcctccaacttcaaaactcaccatcagctctacaaaaataactcatgattgggaactctttgattagttcattctctttcttcccatttttttgcattagaaccgaaccgaatcgaattaaactattttggcatttctgtacatattgctttatgtgggcttcccaattgatattggacccaaatgggctctgttataagcccaataagatgtctgtgcagtgtgatgttgggttaagtggaatattattactctctctttttatcaaattctttcgctttttttttttttttaaccattgtcaacgagtaataatttagtatctaatatttattattttttaaatatttaggctatatttaataataattttgtttttgaaaataaagaatattctttcccattttttattagtcaaattccaaaaacaacaagtttttaaaagttactgtttttagttttcaaattttggcttggttttttaaatcattagtaaaaattagataacaaaagaataaattttgagatggaagtagtgtctatagacttcattttcaaaatcgaaaaaaaaaaaaaaatggttaccaaataaggccttagtttttgtgcttttatcttcataataatttagatcaaatttggtaactatttggttttgggtttttaattgaaaattaagcttataaacatcctttttatctttaaatttcttgttttgttatctactttccaccaacattttaaaaaataaagttattttttgaaaagtaaaaggaaataatttttaaaacttatttttgtttgtaaaatttagctaaaactcatttacttcataaatgtattgaaaatcatagtaaaaaattacgagaaaatatgtttaattttcaaaaacgaaaaatcaaatagtttttaatagttccttagcctctttatttttatttttttaataccatatctctaaattttagtatgtaaccatttagtattcttttaaactttgaatttttaaacaagttattattcattagattctaaagaaatttcttctatatgtaaatagataaaacaatttaatgatgaaatgcatttataaattacaatttattagattgttacaaaaaaaaaaaaaagaaaagaaaaaaaaatagttcacacttgcttgcaatggaatttttatatcagggtatggatgtaaattgttacaaacacagttaatcattgtttgcctttaatattttcaattatacatagaaagttggctcatacattacctttctcaaacatgttatttatggcaacttcttagtttactctcttcctctctatttctttgcctttcctctaccattaagactcctcttgttattttcaaagactatttaatttaattaaataacgctaatgagttttaataattatctaattaatattataacgttttcgttttactgatctcttaattttagaagaataaggacttcaatcaatagttatatatttgttaaaaatctattgatccaatcttttataaataaaacaagtccaaaattaaacaagaaagatcgatgatattatcgaacactttgaaataattatgaactttttaataagttaatgtagatatgttttaaatataagaagggccatgctttacatggtatcaactttaagtctcatgcaaatattgcaactcatgggtgtacaaagatagatcaacaagcatatttatcaattttttaaatttaaaaacaaagttcatttctttaattttcataatcatagggtttataaaaaggctacatagtccctaccaattcattcattatttcttccctttggctaaggtacgtacatacatttaactatataaaatgattttttttcaaaactatgatatacataaccatatatatattttttgccattttgttttttaggcatcttctaatcaatctcatggaggaaagttacaaaatgaggatgaaccaaggcggaggaattccgacgatggcagcagcactgccaccattgccaccgtcatgtttaggaaaactgacaactagcggtgaaaaaaagctaccgttctttcaatccaacatgaatttaagtatgtatggaaatgacaaaagtatcctttctcaaagagaagctaccataacaccaccgccgaagcaacaccaatcacaacttctagactccgacaaagatctcactgtcgaagccaagcgactaagaaggttcactctcttgaaactaatctagaacttaaaagtaaaatttaaaaagtcatttcaaacaaccctaacgttcttttattcatgtacgtacgtaaatacgtacataaatgtttttttttttttttttacatgggttaattttttattttttactatagggtgatgcaaagcagacaatactctcaaaagtatcgactaaaacagcttcattatattactcagcttgaatcagaactaaaagcccttcaagtaattacataaatcaataaataatatcaatcaattaattaattaattaaattagttgattaatcaccttttttggttgaacgaattaaaacaggcagaagtaacaattaccacaccgaggataaaattcatggaccgtcaaaattcactgctccgagccgaaaattactccatcaaagagaaattatctgcatacaccggagaacttctattcaaagaaggtaaattaaattaaaatttttattttatttttaaaaattttaatatgatatttttgcatggggcagctcaatacgaagaattgaaaagagagagaaatatgcttaaggaaatctacgaagcatatcagttaaaattgctggagactctgaaaagcagcaacaacaacaacaacacaactgctgccagtggaagcacttttcaattggtcgaaaattacccacaaattgctaccaaatcaaacccatttaccatgctcgaaaattaaattaaattatcaaaatcaacaataagacattttgaaatttgtaatagttaattaaaccatggtgagatggatttagttgttgtcatttctaagatatatatgtgtgtgtgtgtatgtttgaaatatatttttcctatgtaatttgtagggttgaatttgaagggaacttttgttcaatttgtaattcaatttctgatttttctttctatattaaattatgtcacattttaatgtttagggctggcatgagatgatgaaaggctatatatgcatgtatattcataaatttgtttccttaagaaatttgatgatgctggacattggataagactaattggcagcctgatcatatgcttcaatcaatatttctataatggaataagcaaattggtaagtgtgtggcctcctggcatggttgtggacgtgattggcgaaatcaaaaggtggggagacacatctcatactccatttgccaaaggttgagcaattagctcgttactcactgccttacccatcaaccatgctttggtgtgagctttcagctttcagctttcagctttgttatttacaatatatatttcctctctttcaactgctccatcttcttctgtgatccttactttcctttatgattgtataatgagaatgtttggaaaatcgtaataagatagacttgtaatgtaatgtaatccaaaattaatgtttggattgaacgttttggacccgatttgtaatacgaaagtcattctgttccgacagttgtcaacccaatcctatttttcatcgttttcatggttcacaatctcattttcgtttatttcttaaatactcacacattccaatactccactaatttccaataatcctattacattccacctcccccttaattctctttaacatactagtttgcacatagttaaaaaattttagaatataaaattttaaatgaatatgcttttatttaaatttaaatagtaaattttttacgtgagatttaaaagtaatgaaaaatataatatatatgtatatatatatatatatataatttggttagttttttagaagggaaaacaaaattgtttagatacataaaaagttaaaacatatgattcttacaccgtactaattttctaaacaaccaaaaaggaatccaaaactttatattaaaatataaaaatcttcaaaatttccgcttaaaactcagcaaaacaattaatagcaaaatataaaaaaaatgcctaccaaaaaatatcatatttgatcctgataatttttttaattgatcatagcaagcaaactaatttaaattgtaaaaatgatcaacaaagtctcctcatcgaaaagtgttgggtcatctattaaattagagggagagaggaaataaaagattgaggtgaaatgggagggtagatagcagctttcatctatatactatgctaaggacatattttaatttttctttaatgtttaagtgcattattgaaactttaaaagtttcagaggtattttttttaaaaaaaaaaattagtagtttttgttaaaaactaacaacaatcgtatttttgaaattttttttaaacttaaagttattaatgaaattttggaaagtttatggttatttttaaaataaaaaaattgtatatcttctataatttatcttgaattttctttgtttacaaatttgattttacatgtttcaagtagcatttttaagtggcatggtcaaatgtttaaaatataccatgcaaatgaaagttgtttaatttagctaaattaaacttatcaaaatcaaacgttatttaaatttcactattctttttataatatgtgtgataggaaaatagaacttctcaccaaaatgttgatgtacaaatttgatgagtttgaaaaatttaactaattacaactaatggtaagattcaacttcttaccctagcttcttacttctttgaaagtatgaaattatatacataaaaagacaaactaatttgctaagtcttccaaaataaaccataatattttaatttatttcatctcaattta

SEQ ID NO: 14 - protein, 200 aa, Citrullus lanatus
  CL08381_WT_allele
MACCAATGTCGGCGGCTGATGACGTCCGTCACGAAATGCATCATARAGTGACGAACTTTTATCCGTGTAGATTTTTGGTATTTCCATCCCTTGCGGAGCCGTCATAATTCCAAAACGGCAATGCAAAATCAGGATCCTTAATCAAAGACCCCAATATTCTCTCATGAAAGTAAAGATAAAAACGATGGAATGGGAAGAAC

SEQ ID NO: 15 - protein, 201 aa, Citrullus lanatus
  CL08381_MUT_allele
MACCAATGTCGGCGGCTGATGACGTCCGTCACGAAATGCATCATARAGTGACGAACTTTTATCCGTGTAGATTTTTGGTATTTCCATCCCTTGCGGAGCCAGTCATAATTCCAAAACGGCAATGCAAAATCAGGATCCTTAATCAAAGACCCCAATATTCTCTCATGAAAGTAAAGATAAAAACGATGGAATGGGAAGAAC

SEQ ID NO: 16 - DNA, 19 bp, Citrullus lanatus
  CL_chr2_gap_F1_ primer
agagtgaaccaaaagatcc

SEQ ID NO: 17 - DNA, 20 bp, Citrullus lanatus
  CL_chr2_gap_R3_primer
cccaaaaccaaatagttacc

SEQ ID NO: 18 - DNA, 18 bp, Citrullus lanatus
  CL_chr2_gap_F2 primer
gaaccaaaagatccacca

SEQ ID NO: 19 - DNA, 19 bp, Citrullus lanatus
  CL_chr2_gap_R1 primer
acctacatccactccctaa

SEQ ID NO: 20 - DNA, 2744 bp, Citrullus lanatus
  PPO_WT-gDNA reverse complementary sequence
ttatgcatcatattcaattctaatgtccttaacggtggcggacccatctccaaacctagggaccaatgtaacaataatgctatcgtcgttatccgcatccaaactctcaagcagttcagttatccctaacctaaggcatgtttttatgttcatgctgctgctacctttcatatgaggcacattcacaaagctccctgcaaactcagaattatccgctctaatttccctatcatcctcgtcattgataaaaacatcaaacttaatagccttgtttccgtcgaactcaatcccatcaatcaccaaaatctcctcttcatcgtctttctccttcgtacccctcgatttcttcggcctcttgacttcgaagctgacgatcttgtcaacactcgacggtagcttcccggtcttcttggtagatttcttcttggttttgttgggtgtgcgtggtactcgtggggttggaggtgttttgagccatggaattggtacggtgtcatcgtagacatagcctaaggctctggtatctagacagtctttgacataaactcggacagcttcaccattctcatcgtagaatacaaaggaagcgtttaggaaatctttgtctttaatgtcttgtcgcttttcgcctaaggatttccatatggaccaaaaacggtccacgttcgcgtggtgggcgtagaagatgggatctctagccgctgagaagaaggttcccatgtcaattcggttcgattggttcgggtcacccgtccacaaatgaattgaattgtgaggaaggttttccaccgtccccattcctttcaaaaaattaatcaattcatcaaaattacatcattttactttccatactaatatccttaattattatctctaaacatcattatctaatttacaaatgggcatgtttagaatacattttcaaatgattaaattaaaaaaacaagtcaatttgatgaccagcataacttatcaacacaacccaatcatcactgtttcgattgggttaggttgagttcaaataaattaaaaagttattggttgagttgtttatgtgttctcttcaaataaccccaacttgttataattcttttataaaagatttacttatggtacaattatatatacatatatttattttaattttatttaatttcataattttttggaaagtttattctccagaaacttcaaacaataatattttagcattttgaaaagaaaatcttcataatataaattgaaattgagttgttaattttaattcaatatataaaaataattcaacaaaaattttacatattaacattttagggttgttttcaaatatagcaaaaaaagtcaaattatttgcaaatatttcattgtctatccgtgataaaccgcgataaacttttatcattaggggatagactcaattaaatctttctgtatttgtaaaaactttgatttttttccatttataatagtttcctaacattttacttatttttagaacagaaatttacataaatgacctaagtaatttgaacccatatatttcatgattgagttgaattcgattcattatttaatagaatttatttgaattgaaaaaacttatcaatccgacaattgagttagatctataaactgctctaatccaacccaaaaacactcttaacattaattttcaaaaactaaaaacattacctaacataaccataactatatatgattgtcttttttatcttttcctttttttgaaaaaagaaattatatggttgaaaaaaaaaatcacctggacttgggttgctgccacttcgataaggctggccgaaaaagagcaagggcgtacgggcgccggacacgacctggcgatacataacacttagattgcattggattatcttttctctgcttattgttggctcaacgtcattgtaatccaaatcaaccaatgtcggcggctgatgacgtccgtcacgaaatgcatcatagagtgacgaacttttatccgtgtagatttttggtatttccatcccttgcggagcgtcataattccaaaacggcaatgcaaaatcaggatccttaatcaaagaccccaatattctctcatgaaagtaaagataaaaacgatggaatgggaagaacagccacgagaaatgaacttgtaattcaactggaagacccaattgatcgtaacccccagtacaataagcacagtgaacaagtgcttgctgtttaaaactacgtggatcatcatcaggaagcgctttcataagcgctacggcttccttatacttttcaatatattctttatctaatgattgtgccgctttcctaacgcgtggtttgaggaagggttttacgttattggtggatggtgggcagcaaaccaaatctttgacgccatctgccaagtccgtgcttgatccacacttggaagggtcgggggttgtgactggagctgccaaagcgaagggatcaactccaaaagcacttgaagctgagccatacagaccgccgagcccgataagcgcttctctccggtcaacaaacttgcctggccataatgagttattactttcttcaccactgccattggagccgctacacacaaccaagttattgagtctatgaatggtggaagatggatctttttttttacgataaaacagaccaaaggaggcgccgccggtggtggccgtggttattgcggcggaggaaagtgctagtggcatggaaggagatagagaggccat